(19) United States
(12) Patent Application Publication
(10) Pub. No.: US 2023/0333113 A1
(43) Pub. Date: Oct. 19, 2023

(54) DETECTION OF AN ANALYTE OF INTEREST BY NANOESI MASS SPECTROMETRY

(71) Applicant: Roche Diagnostics Operations, Inc., Indianapolis, IN (US)

(72) Inventors: Dieter Heindl, Muenchen (DE); Uwe Kobold, Weilheim (DE); Martin Rempt, Penzberg (DE); Christoph Zuth, München (DE)

(21) Appl. No.: 18/306,195

(22) Filed: Apr. 24, 2023

Related U.S. Application Data
(63) Continuation of application No. PCT/EP2021/079018, filed on Oct. 20, 2021.

(30) Foreign Application Priority Data
Oct. 22, 2020 (EP) 20203220.7

(51) Int. Cl. G01N 33/58; G01N 33/74

(52) U.S. Cl. CPC G01N 33/58; G01N 33/743; G01N 2560/00

(57) ABSTRACT

The present invention relates to a method, a diagnostic system, a kit and the use thereof for efficiently detection of an analyte of interest by nanoESI mass spectrometry.

### FIELD OF THE INVENTION

The present invention relates to a method, a diagnostic system, a kit and the use thereof for efficiently detection of an analyte of interest by nanoESI mass spectrometry.

### BACKGROUND OF THE INVENTION

Mass spectrometry (MS) is a widely used technique for the qualitative and quantitative analysis of chemical substances ranging from small molecules to macromolecules. In general, it is a very sensitive and specific method, allowing even for the analysis of complex biological, for example (e.g.), environmental or clinical samples. However, for several analytes, especially if analysed from complex biological matrices such as serum, sensitivity of the measurement remains an issue.

Often MS is combined with chromatographic techniques, particularly gas and liquid chromatography such as e.g. HPLC. Here, the analysed molecule (analyte) of interest is separated chromatographically and is individually subjected to mass spectrometric analysis (Higashi et al. (2016) J. of Pharmaceutical and Biomedical Analysis 130 p. 181-190).

To ensure reliable and sensitive mass spectrometric detection (avoiding matrix effects and interference as well as increasing sensitivity) it is necessary to separate chromatographically the target analytes as well as possible. In general, this can be done by isocratic or gradient systems, for example, reversed phase HPLC columns and gradients from aqueous to organic phases. The columns used for HPLC require flow rates between 0.1 and 1.0 ml/min. Under these optimal flow conditions, very narrow chromatographic peaks with very small peak volumes are produces.

Nano-ESI (Nano—electrospray ionization) is known for its high sensitivity due to the fact that matrix effects, i.e. competitive reactions for charges as M⁺H, do not occur. A high degree of the analyzed molecules enters the MS system more effectively due to the better distribution in a spray and disturbing neutral particles (residual solvents) are minimized.

There is, however, still a need of increasing the sensitivity of MS analysis methods, particularly for the analysis of analytes that have a low abundance or when only little materials (such as biopsy tissues) are available.

In modern, high-resolution HPLC separation systems with optimal flow conditions, very narrow chromatographic peaks with very small peak volumes are generated. These optimal flow conditions have typically flow rates of 0.1 to 1.0 ml/min. Thus, these flow rates are compatible with so-called “normal flow ESI” ion sources. However, an ESI under these conditions has the disadvantage that the ion yield for the target analyte is highly dependent on the composition of the accompanying substances which are present in the ion source together with the analyte (matrix effects). Furthermore, it is known that only a small part of the analyte molecule is ionized in the process and is then used for mass spectrometric analysis.

Although diluting the sample with solvent leads to a reduction of matrix effects, the dilution also leads to a deterioration of the detection limits.

When using so-called nano-ESI sources (flow rates of less than 1 μl/min, typically 50 nL to 200 nL/min) the ion yields are improved, but due to the low flow rate only a very small sample volume can be applied, which in turn adversely affects the detection limits.

Through derivatization reactions, the detection limits can be improved, in particular more than a factor of 100 is possible.

For chemically induced derivatization reactions, auxiliary reagents (derivatization agents/catalysts or similar) must always be used which can interfere with the ionization, because these auxiliary reagents are contained in very high excess in relation to the analyte.

There is thus an urgent need in the art for a method which allows for a sensitive detection of analytes from complex biological matrices as well as exhibiting a chemical structure which does not negatively influence the MS measurement workflow. This is of particular importance in a random-access, high-throughput MS set up, wherein several different analytes exhibiting different chemical properties have to be measured in a short amount of time.

The present invention relates to a method of determining the level of an analyte of interest in a pretreated sample which allows for a sensitive determination of analyte molecules such as steroids, proteins, and other types of analytes, in biological samples. The reagent is designed in a modular manner to allow the individual adaption for specific needs arising in the measurement of certain analytes or for specific workflow adaptations.

It is an object of the present invention to provide a method, a diagnostic system, a kit and the use thereof for efficiently detection of an analyte of interest by nanoESI mass spectrometry.

This object is or these objects are solved by the subject matter of the independent claims. Further embodiments are subjected to the dependent claims.

### SUMMARY OF THE INVENTION

In the following, the present invention relates to the following aspects:

In a first aspect, the present invention relates to a method of determining the level of an analyte of interest in a pretreated sample comprising the following steps:
a) Providing the pretreated sample, in particular the pretreated sample of bodily fluid including the analyte of interest.
b) Derivatizing the analyte of interest, preferably in the pretreated sample,
c) Diluting the pretreated sample, and
d) Determining the level of the analyte of interest in the pretreated sample using nanoESI mass spectrometry.

In a second aspect, the present invention relates to the use of the method of the first aspect of the present invention for determining the level of an analyte of interest in a pretreated sample.

In a third aspect, the present invention relates to a diagnostic system for determining a level of an analyte of interest in a pretreated sample.

In a fourth aspect, the present invention relates to the use of the diagnostic system of the third aspect of the present invention in the method of the first aspect of the present invention.

In a fifth aspect, the present invention relates to a kit suitable to perform a method of the first aspect of the present invention comprising
(i) a compound for derivatizing the analyte of interest in a pretreated sample, wherein the compound is capable of forming a covalent bond to the analyte of interest,
(ii) a solvent or mixtures of solvents for diluting the pretreated sample comprising the derivatized analyte of interest, and
(iii) optionally a catalyst.

In a sixth aspect, the present invention relates to the use of a kit of the fifth aspect of the present invention in a method of the first aspect of the present invention.

### LIST OF FIGURES

FIG. 1A shows two methods of determining the level of analyte of interest in a neat solution, in this case of testosterone as the analyte of interest. FIG. 1B shows relative intensity as a function of the concentration of underivatized Testosterone and derivatized Testosterone in the neat solution. As a derivatizing reagent Girard T and Mz2974 were used.

FIG. 2A shows two methods of determining the level of analyte of interest in horse serum, in this case of testosterone as the analyte of interest. FIG. 2B shows relative intensity as a function of the concentration of underivatized Testosterone and derivatized Testosterone in horse serum. As a derivatizing reagent Girard T and Mz2974 were used.

FIG. 3A shows the method of determining the level of the analyte of interest comprising the derivatizing and dilution step in a bead eluat and depletion horse serum. FIG. 3B shows the results of the method according to FIG. 3A.

FIG. 4 shows an enrichment step according to the present invention.

FIGS. 5 to 7 and 10 show the area ratio as a function of the concentration in ng/ml of a ¹³C3-Testosterone and the derivatives thereofs according to a comparative example or an example of the present invention.

FIG. 8A to 13B show the comparison of static nanoESI (Nanomate, ˜0.5 μL/min) and static ESI (direct injection, 100 μL/min) of an (derivatized) analyte of interest in depl. horse serum according to a comparative example or an example of the present invention.

FIG. 14 to 16C show calibration curves.

### DETAILED DESCRIPTION OF THE INVENTION

Before the present invention is described in detail below, it is to be understood that this invention is not limited to the particular embodiments and examples described herein as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art.

Several documents are cited throughout the text of this specification. Each of the documents cited herein (including all patents, patent applications, scientific publications, manufacturer's specifications, instructions etc.), whether supra or infra, is hereby incorporated by reference in its entirety. In the event of a conflict between the definitions or teachings of such incorporated references and definitions or teachings recited in the present specification, the text of the present specification takes precedence.

In the following, the elements of the present invention will be described. These elements are listed with specific embodiments, however, it should be understood that they may be combined in any manner and in any number to create additional embodiments. The various described examples and preferred embodiments should not be construed to limit the present invention to only the explicitly described embodiments. This description should be understood to support and encompass embodiments which combine the explicitly described embodiments with any number of the disclosed and/or preferred elements. Furthermore, any permutations and combinations of all described elements in this application should be considered disclosed by the description of the present application unless the context indicates otherwise.

### Definitions

The word “comprise”, and variations such as “comprises” and “comprising”, will be understood to imply the inclusion of a stated integer or step or group of integers or steps but not the exclusion of any other integer or step or group of integers or steps. The term “including” and “comprising” can be used interchangeable.

As used in this specification and the appended claims, the singular forms “a”, “an”, and “the” include plural referents, unless the content clearly dictates otherwise.

Percentages, concentrations, amounts, and other numerical data may be expressed or presented herein in a “range” format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “4% to 20%” should be interpreted to include not only the explicitly recited values of 4% to 20%, but to also include individual values and sub-ranges within the indicated range. Thus, included in this numerical range are individual values such as 4, 5, 6, 7, 8, 9, 10 . . . 18, 19, 20% and sub-ranges such as from 4-10%, 5-15%, 10-20%, etc. This same principle applies to ranges reciting minimal or maximal values. Furthermore, such an interpretation should apply regardless of the breadth of the range or the characteristics being described.

The term “about” when used in connection with a numerical value is meant to encompass numerical values within a range having a lower limit that is 5% smaller than the indicated numerical value and having an upper limit that is 5% larger than the indicated numerical value.

In the context of the present invention, the term “compound” or “derivatisation reagent” or “label” are used interchangeably and refer to a chemical substance having a specific chemical structure. Said compound may comprise one or more reactive groups. Each reactive group may fulfil a different functionality, or two or more reactive groups may fulfil the same function. Reactive groups include but are not limited to reactive units, charged units, and neutral loss units.

The term “Mass Spectrometry” (“Mass Spec” or “MS”) or “mass spectrometric determination” or “mass spectrometric analysis” relates to an analytical technology used to identify compounds by their mass. MS is a methods of filtering, detecting, and measuring ions based on their mass-to-charge ratio, or “m/z”. MS technology generally includes (1) ionizing the compounds to form charged compounds; and (2) detecting the molecular weight of the charged compounds and calculating a mass-to-charge ratio. The compounds may be ionized and detected by any suitable means. A “mass spectrometer” generally includes an ionizer and an ion detector. In general, one or more molecules of interest are ionized, and the ions are subsequently introduced into a mass spectrographic instrument where, due to a combination of magnetic and electric fields, the ions follow a path in space that is dependent upon mass (“m”) and charge (“z”). The term “ionization” or “ionizing” refers to the process of generating an analyte ion having a net charge equal to one or more units. Negative ions are those having a net negative charge of one or more units, while positive ions are those having a net positive charge of one or more units. The MS method may be performed either in “negative ion mode”, wherein negative ions are generated and detected, or in “positive ion mode” wherein positive ions are generated and detected.

“Tandem mass spectrometry” or “MS/MS” involves multiple steps of mass spectrometry selection, wherein fragmentation of the analyte occurs in between the stages. In a tandem mass spectrometer, ions are formed in the ion source and separated by mass-to-charge ratio in the first stage of mass spectrometry (MS1). Ions of a particular mass-to-charge ratio (precursor ions or parent ion) are selected and fragment ions (or daughter ions) are created by collision-induced dissociation, ion-molecule reaction, or photodissociation. The resulting ions are then separated and detected in a second stage of mass spectrometry (MS2).

Since a mass spectrometer separates and detects ions of slightly different masses, it easily distinguishes different isotopes of a given element. Mass spectrometry is thus, an important method for the accurate mass determination and characterization of analytes, including but not limited to low-molecular weight analytes, peptides, polypeptides or proteins. Its applications include the identification of proteins and their post-translational modifications, the elucidation of protein complexes, their subunits and functional interactions, as well as the global measurement of proteins in proteomics. De novo sequencing of peptides or proteins by mass spectrometry can typically be performed without prior knowledge of the amino acid sequence.

Most sample workflows in MS further include sample preparation and/or enrichment steps, wherein e.g. the analyte(s) of interest are separated from the matrix using e.g. gas or liquid chromatography. Typically, for the mass spectrometric measurement, the following three steps are performed:
1. a sample comprising an analyte of interest is ionized, usually by complex formation with cations, often by protonation to cations. Ionization source include but are not limited to electrospray ionization (ESI), nano electrospray ionization (nanoESI) and atmospheric pressure chemical ionization (APCI).
2. the ions are sorted and separated according to their mass and charge. High-field asymmetric-waveform ion-mobility spectrometry (FAIMS) may be used as ion filter.
3. the separated ions are then detected, e.g. in multiple reaction mode (MRM), and the results are displayed on a chart.

The term “electrospray ionization” or “ESI,” refers to methods in which a solution is passed along a short length of capillary tube, to the end of which is applied a high positive or negative electric potential. Solution reaching the end of the tube is vaporized (nebulized) into a jet or spray of very small droplets of solution in solvent vapor. This mist of droplets flows through an evaporation chamber, which is heated slightly to prevent condensation and to evaporate solvent. As the droplets get smaller the electrical surface charge density increases until such time that the natural repulsion between like charges causes ions as well as neutral molecules to be released.

The term “nano electrospray ionization” or “nanoESI” refers to methods typically using flow rates below 1 μL/min either in static or dynamic mode. Preferably, nanoESI uses a flow rate of 50 to 500 nl/min, e.g. 500 nl/min. 500 nl/min is equal to 0.5 μl/min.

The term “static nanoESI mass spectrometry” is used in the context of the present disclosure as a non-continuous flow nanoESI option. The analysis is typically defined by a discrete sample being loaded by single-use pipette tips into an emitter. In contrast, dynamic nanoESI mass spectrometry is characterized by a mobile phase pumped at low flow rates through a small diameter emitter.

The term “atmospheric pressure chemical ionization” or “APCI,” refers to mass spectrometry methods that are similar to ESI: however, APCI produces ions by ion-molecule reactions that occur within a plasma at atmospheric pressure. The plasma is maintained by an electric discharge between the spray capillary and a counter electrode. Then ions are typically extracted into the mass analyzer by use of a set of differentially pumped skimmer stages. A counterflow of dry and preheated Ni gas may be used to improve removal of solvent. The gas-phase ionization in APCI can be more effective than ESI for analyzing less-polar entity.

“High-field asymmetric-waveform ion-mobility spectrometry (FAIMS)” is an atmospheric pressure ion mobility technique that separates gas-phase ions by their behavior in strong and weak electric fields.

“Multiple reaction mode” or “MRM” is a detection mode for a MS instrument in which a precursor ion and one or more fragment ions are selectively detected.

Mass spectrometric determination may be combined with additional analytical methods including chromatographic methods such as gas chromatography (GC), liquid chromatography (LC), particularly IIPLC, and/or ion mobility-based separation techniques.

In the context of the present disclosure, the term “analyte”, “analyte molecule”, or “analyte(s) of interest” are used interchangeably referring the chemical species to be analysed via mass spectrometry, in particular nanoESI mass spectrometry. Chemical species suitable to be analysed via mass spectrometry, i.e. analytes, can be any kind of molecule present in a living organism, include but are not limited to nucleic acid (e.g. DNA. mRNA, miRNA, rRNA etc.), amino acids, peptides, proteins (e.g. cell surface receptor, cytosolic protein etc.), metabolite or hormones (e.g. testosterone, estrogen, estradiol, etc.), fatty acids, lipids, carbohydrates, steroids, ketosteroids, secosteroids (e.g. Vitamin D), molecules characteristic of a certain modification of another molecule (e.g. sugar moieties or phosphoryl residues on proteins, methyl-residues on genomic DNA) or a substance that has been internalized by the organism (e.g. therapeutic drugs, drugs of abuse, toxin, etc.) or a metabolite of such a substance. Such analyte may serve as a biomarker. In the context of present invention, the term “biomarker” refers to a substance within a biological system that is used as an indicator of a biological state of said system.

The term “permanent charge” or “permanent charged” is used in the context of the present disclosure that the charge, e.g. a positive or negative charge, of a unit is not readily reversible, for example, via flushing, dilution, filtration, and the like. A permanent charge may be the result, for example, of covalently bonding. A reversible charge (a non-permanent charge) may be the result in contrast to a permanent charge, for example, of an electrostatic interaction.

The term “permanent net charge” or “net charged” is used in the context of the present disclosure that the permanent net charge is the total permanent charge an ion or molecule has. Permanent net charge can be calculated as follows: number of protons—number of electrons=permanent net charge. A permanent net charge can be seen as a covalent combination of atoms which forms by bond rearrangements a charged mojety in the molecule (e.g. quarternary nitrogen, tetramethylammonium) while a net charge can also exist by addition or the abstraction of atoms e.g. hydrogen to result in a pseudomolecular ion consisting of [M⁺H]⁺ or [M⁻H]⁻. For Example, if the compound hast wo permanent positive charges and one permanent negative charge, then the permanent net charge is +1 (2*(+1)+(−1)=(+1)).

The term “compound is capable of covalently binding to the analyte” means that the compound is suitable to bind to the analyte. The binding between the compound and the analyte is covalent.

The term “mass”, for example, m1, m2, m3, m4 or mx with x>4, represents the atomic mass, in particular the unified atomic mass. The unit of the unified atomic mass is u. In the biomedical field Dalton [Da] instead of the unified atomic mass [u] can be used. The Dalton is not an SI unit. The dalton is equivalent to unified atomic mass in that there is no conversion factor between these units. A “mass spectrum” is the two-dimensional representation of signal intensity (ordinate) versus m/z (abscissa). The position of a peak, as signals are usually called, reflects the m/z of an ion that has been created from the compound, analyte or combinations thereof (complex) within the ion source. The intensity of this peak correlates to the abundance of that ion. Often but not necessarily, the peak at highest m/z results from the detection of the intact ionized molecule, the molecular ion, M⁺. The molecular ion peak is usually accompanied by several peaks at lower or higher m/z caused by fragmentation of the compound, analyt or complex to yield fragment ions. Consequently, the respective peaks in the mass spectrum may be referred to as fragment ion peaks or daughter ion peaks. m/z is dimensionless by definition. The term “fragmentation” can mean that the compound, analyt and/or complex is dissociated and form ions, e.g. at least one daughter ion, by passing the compound, analyt and/or complex in the ionization chamber of a mass spectrometer. The fragments cause a unique pattern in the mass spectrum. The term “fragmentation” can refer to the dissociation of a single molecule into two or more separate molecules.

As used herein, the term fragmentation refers to a specific fragmentation event, wherein the breaking point in the parent molecule at which the fragmentation event takes place is well defined, and wherein the two or more daughter molecules resulting from the fragmentation event are well characterised. It is well-known to the skilled person how to determine the breaking point of a parent molecule as well as the two or more resulting daughter molecules. The resulting daughter molecules may be stable or may dissociate in subsequent fragmentation events. Exemplified, in case a parent molecule undergoing fragmentation comprises a N-benzylpyridinium unit, the skilled person is able to determine based on the overall structure of the molecule whether the pyridinium unit will fragment to release an benzyl entity or would be released completely from the parent molecule, i.e the resulting daughter molecules would either be an benzyl molecule and a parent molecule lacking of benzyl.

Fragmentation may occur via collision-induced dissociation (CID), electron-capture dissociation (ECD), electron-transfer dissociation (ETD), negative electron-transfer dissociation (NETD), electron-detachment dissociation (EDD), photodissociation, particularly infrared multiphoton dissociation (IRMPD) and blackbody infrared radiative dissociation (BIRD), surface-induced dissociation (SID), Higher-energy C-trap dissociation (HCD), charge remote fragmentation.

The term “m1/z1<m2/z2” means that the mass-to-charge ratio of the compound (m1/z1) is smaller than the mass-to-charge ratio of at least one or exact one daughter ion of the compound (m2/z2).

The term “limit of detection” or “LOD” is the lowest concentration of an analyte that the bioanalytical procedure can reliably differentiate the analyte from background noise.

The term “signal-to-noise ratio” or S/N describes the uncertainty of an intensity measurement and provides a quantitative measure of a signal's quality by quantifying the ratio of the intensity of a signal relative to noise.

Analytes may be present in a sample of interest, e.g. a biological or clinical sample. The term “sample” or “sample of interest” are used interchangeably herein, referring to a part or piece of a tissue, organ or individual, typically being smaller than such tissue, organ or individual, intended to represent the whole of the tissue, organ or individual. Upon analysis a sample provides information about the tissue status or the health or diseased status of an organ or individual. Examples of samples include but are not limited to fluid samples such as blood, serum, plasma, synovial fluid, spinal fluid, urine, saliva, and lymphatic fluid, or solid samples such as dried blood spots and tissue extracts. Further examples of samples are cell cultures or tissue cultures.

A “covalent bond” or “covalently linked” or “covalently bonded” is at least one chemical bond that involves the sharing of electron pairs between atoms or molecules. e.g. between the compound and the analyte.

The terms “compound” and “label” can be used interchangeable.

Numerical values, e.g. 1, 2, 3, 4, 5 or 6, for the charges, e.g. z1, z2, z3, z4 or zx with x>4, are absolute values of the charges. For example, net charge z1=2 can mean that the net charge z1 is +2 or the net charge is −2. Preferably, the charges in this case are positive numerical values, e.g. 2=+2.

In this context “level” or “level value” encompasses the absolute amount, the relative amount or concentration as well as any value or parameter which correlates thereto or can be derived therefrom.

The term “determining” the level of the analyte of interest, as used herein refers to the quantification of the analyte of interest, e.g. to determining or measuring the level of the analyte of interest in the pretreated sample. The level of the analyte of interest is determined by nanoESI mass spectrometry.

In this context “pretreated sample” refers to a sample, which is prepared for the mass spectrometry, in particular the nanoESI mass spectrometry. In particular, pretreated sample is a sample, which is provided and/or prepared before step (a) and/or (b) of the method is performed. Before the analyte is being analysed via Mass Spectrometry, a sample may be pre-treated in a sample- and/or analyte specific manner. In the context of the present disclosure, the term “pre-treatment” or “pre-treated” refers to any measures required to allow for the subsequent analysis of a desired analyte via Mass Spectrometry, in particular NanoESI Mass Spectrometry. Pre-treatment measures typically include but are not limited to the elution of solid samples (e.g. elution of dried blood spots), addition of hemolizing reagent (HR) to whole blood samples, and the addition of enzymatic reagents to urine samples. Also the addition of internal standards (ISTD) is considered as pre-treatment of the sample. In particular, pre-treatment of the sample does not include enrichment step. e.g. by using magnetic or paramagnetic beads.

The term “hemolysis reagent” (HR) refers to reagents which lyse cells present in a sample, in the context of this invention hemolysis reagents in particular refer to reagents which lyse the cell present in a blood sample including but not limited to the erythrocytes present in whole blood samples. A well known hemolysis reagent is water (H2O). Further examples of hemolysis reagents include but are not limited to deionized water, liquids with high osmolarity (e.g. 8M urea), ionic liquids, and different detergents.

Typically, an “internal standard” (ISTD) is a known amount of a substance which exhibits similar properties as the analyte of interest when subjected to the mass spectrometric detection workflow (i.e. including any pre-treatment, enrichment and actual detection step). Although the ISTD exhibits similar properties as the analyte of interest, it is still clearly distinguishable from the analyte of interest. Exemplified, during a chromatographic separation, such as gas or liquid chromatography, the ISTD has about the same retention time as the analyte of interest from the sample. Thus, both the analyte and the ISTD enter the mass spectrometer at the same time. The ISTD however, exhibits a different molecular mass than the analyte of interest from the sample. This allows a mass spectrometric distinction between ions from the ISTD and ions from the analyte by means of their different mass/charge (m/z) ratios. Both are subject to fragmentation and provide daughter ions. These daughter ions can be distinguished by means of their m/z ratios from each other and from the respective parent ions. Consequently, a separate determination and quantification of the signals from the ISTD and the analyte can be performed. Since the ISTD has been added in known amounts, the signal intensity of the analyte from the sample can be attributed to a specific quantitative amount of the analyte. Thus, the addition of an ISTD allows for a relative comparison of the amount of analyte detected, and enables unambiguous identification and quantification of the analyte(s) of interest present in the sample when the analyte(s) reach the mass spectrometer. Typically, but not necessarily, the ISTD is an isotopically labeled variant (comprising e.g. 2H, 13C, or 15N etc. label) of the analyte of interest.

In addition to the pre-treatment, the sample may also be subjected to one or more enrichment steps. In the context of the present disclosure, the term “first enrichment process” or “first enrichment workflow” refers to an enrichment process which occurs subsequent to the pre-treatment of the sample and provides a sample comprising an enriched analyte relative to the initial sample. The first enrichment workflow may comprise chemical precipitation (e.g. using acetonitrile) or the use of a solid phase. Suitable solid phases include but are not limited to Solid Phase Extraction (SPE) cartridges, and beads. Beads may be non-magnetic, magnetic, or paramagnetic. Beads may be coated differently to be specific for the analyte of interest. The coating may differ depending on the use intended, i.e. on the intended capture molecule. It is well-known to the skilled person which coating is suitable for which analyte. The beads may be made of various different materials. The beads may have various sizes and comprise a surface with or without pores.

In the context of the present disclosure the term “second enrichment process” or “second enrichment workflow” refers to an enrichment process which occurs subsequent to the pre-treatment and the first enrichment process of the sample and provides a sample comprising an enriched analyte relative to the initial sample and the sample after the first enrichment process.

In the context of the present disclosure, the sample may be derived from an “individual” or “subject”. Typically, the subject is a mammal. Mammals include, but are not limited to, domesticated animals (e.g., cows, sheep, cats, dogs, and horses), primates (e.g., humans and non-human primates such as monkeys), rabbits, and rodents (e.g., mice and rats).

The term “serum” as used herein is the clear liquid pan of the blood hat can be separated from clotted blood. The term “plasma” as used herein is the clear liquid pan of blood which contains the blood cells. Serum differs from plasma, the liquid portion of normal unclotted blood containing the red and white cells and platelets. It is the clot that makes the difference between serum and plasma. The term “whole blood” as used herein contains all components of blood, for examples white and red blood cells, platelets, and plasma.

The term “in vitro method” is used to indicate that the method is performed outside a living organism and preferably on body fluids, isolated tissues, organs or cells.

The term “lyophilization” is used to indicate that a product is dried in a low temperature dehydration process, e.g. low temperatures at −10° C. to −40° C., by lowering the pressure and removing the ice by sublimation.

The term “centrifuge” is used to indicate that particles are separated from a solution, suspension and/or dispersion by the application of centrifugal forces. Separation depends on either the size of the particles, the density, the shape, viscosity of the medium and the rotor speed of the centrifuge.

The term “automatically” or “automated” as used herein is a broad term and is to be given its ordinary and customary meaning to a person of ordinary skill in the an and is not to be limited to a special or customized meaning. The term specifically may refer, without limitation, to a process which is performed completely by means of at least one computer and/or computer network and/or machine, in particular without manual action and/or interaction with a user.

The term “diluting” as used herein is a broad term. Diluting can indicate that the level of the analyte of interest in the pretreated sample provided by step (a) or step (b) is greater than the level of the (same) analyte of interest in the pretreated sample provided in or after step (c).

The term “chromatography” refers to a process in which a chemical mixture carried by a liquid or gas is separated into components as a result of differential distribution of the chemical entities as they flow around or over a stationary liquid or solid phase.

The term “liquid chromatography” or “LC” refers to a process of selective retardation of one or more components of a fluid solution as the fluid uniformly percolates through a column of a finely divided substance, or through capillary passageways. The retardation results from the distribution of the components of the mixture between one or more stationary phases and the bulk fluid, (i.e., mobile phase), as this fluid moves relative to the stationary phase(s). Methods in which the stationary phase is more polar than the mobile phase (e.g., toluene as the mobile phase, silica as the stationary phase) are termed normal phase liquid chromatography (NPLC) and methods in which the stationary phase is less polar than the mobile phase (e.g., water-methanol mixture as the mobile phase and C18 (octadecylsilyl) as the stationary phase) is termed reversed phase liquid chromatography (RPLC).

“High performance liquid chromatography” or “HPLC” refers to a method of liquid chromatography in which the degree of separation is increased by forcing the mobile phase under pressure through a stationary phase, typically a densely packed column. Typically, the column is packed with a stationary phase composed of irregularly or spherically shaped particles, a porous monolithic layer, or a porous membrane. HPLC is historically divided into two different sub-classes based on the polarity of the mobile and stationary phases.

Further well-known LC modi include hydrophilic interaction chromatography (HILIC), size-exclusion LC, ion exchange LC, and affinity LC.

LC separation may be single-channel LC or multi-channel LC comprising a plurality of LC channels arranged in parallel. In LC analytes may be separated according to their polarity or log P value, size or affinity, as generally known to the skilled person. The term “reactive unit” refers to a unit able to react with another molecule, i.e. which is able to form covalent bond with another molecule, such as an analyte of interest. Typically, such covalent bond is formed with a chemical group present in the other molecule. Accordingly, upon chemical reaction, the reactive unit of the compound forms a covalent bond with a suitable chemical group present in the analyte molecule. As this chemical group present in the analyte molecule, fulfils the function of reacting with the reactive unit of the compound, the chemical group present in the analyte molecule is also referred to as the “functional group” of the analyte. The formation of the covalent bond occurs in each case in a chemical reaction, wherein the new covalent bond is formed between atoms of the reactive group and the functional groups of the analyte. It is well known to the person skilled in the an that in forming the covalent bond between the reactive group and the functional groups of the analyte, atoms are lost during this chemical reaction.

In the context of the present disclosure, the term “complex” refers to the product produced by the reaction of a compound with an analyte molecule. This reaction leads to the formation of a covalent bond between the compound and the analyte. Accordingly, the term complex refers to the covalently bound reaction product formed by the reaction of the compound with the analyte molecule.

A “kit” is any manufacture (e.g., a package or container) comprising at least one reagent, e.g., a medicament for treatment of a disorder, or a probe for specifically detecting a biomarker gene or protein of the invention. The kit is preferably promoted, distributed, or sold as a unit for performing the methods of the present invention. Typically, a kit may further comprise carrier means being compartmentalized to receive in close confinement one or more container means such as vials, tubes, and the like. In particular, each of the container means comprises one of the separate elements to be used in the method of the first aspect. Kits may further comprise one or more other reagents including but not limited to reaction catalyst. Kits may further comprise one or more other containers comprising further materials including but not limited to buffers, diluents, filters, needles, syringes, and package inserts with instructions for use. A label may be present on the container to indicate that the composition is used for a specific application, and may also indicate directions for either in vivo or in vitro use. The computer program code may be provided on a data storage medium or device such as a optical storage medium (e.g., a Compact Disc) or directly on a computer or data processing device. Moreover, the kit may, comprise standard amounts for the biomarkers as described elsewhere herein for calibration purposes.

### Embodiments

In a first aspect, the present invention relates to a method of determining the level of an analyte of interest in a pretreated sample comprising the following steps:
a) Providing the pretreated sample, in particular the pretreated sample of bodily fluid including the analyte of interest,
b) Derivatizing the analyte of interest, preferably in the pretreated sample, c) Diluting the pretreated sample, and
d) Determining the level of the analyte of interest in the pretreated sample using nanoESI mass spectrometry.

The inventor surprisingly found that a method of the first aspect of the invention allows a sensitive detection of analytes from complex biological matrices as well as exhibiting a chemical structure which does not negatively influence the MS measurement workflow. This is of particular importance in a random-access, high-throughput MS set up, wherein several different analytes exhibiting different chemical properties have to be measured in a short amount of time.

The present invention relates to a method of determining the level of an analyte of interest in a pretreated sample which allows for a sensitive determination of analyte molecules such as steroids, proteins, and other types of analytes, in biological samples. The reagent is designed in a modular manner to allow the individual adaption for specific needs arising in the measurement of certain analytes or for specific workflow adaptations.

Nano-ESI shows advantages with respect to ESI. Nano-ESI (Nano—electrospray ionization) has a high sensitivity and low sample consumption. As a consequence of this remarkably lower sample flow rates there is an affection of the mechanism of ion formation compared to conventional electrospray ionization (ESI). Small droplet sizes cause an improved desolvation and optimized ionization process. Matrix effects. i.e. competitive reactions for charges as M⁺H, are dramatically reduced or do not occur. A high degree of the analyzed molecules enters the MS system more effectively due to the better distribution in a spray, a closer proximity to the MS inlet, and disturbing neutral particles (residual solvents) are minimized.

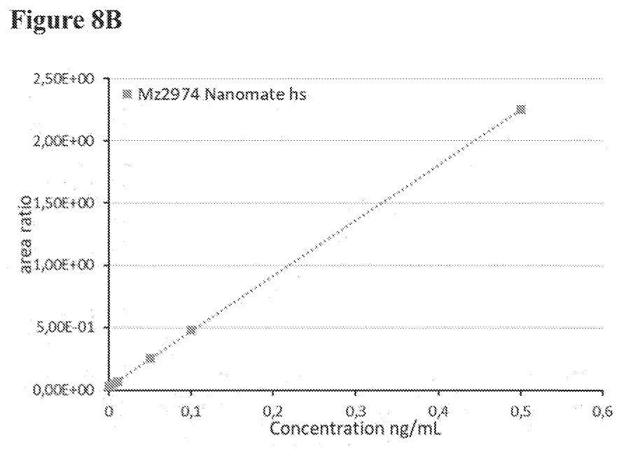

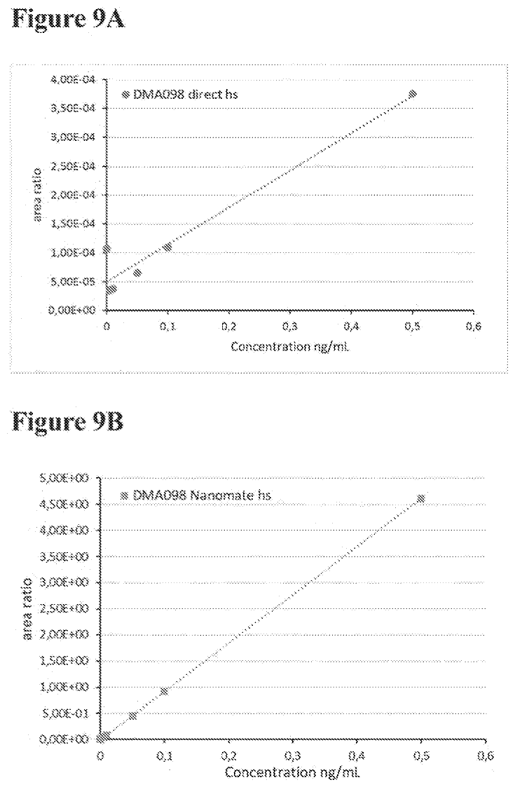

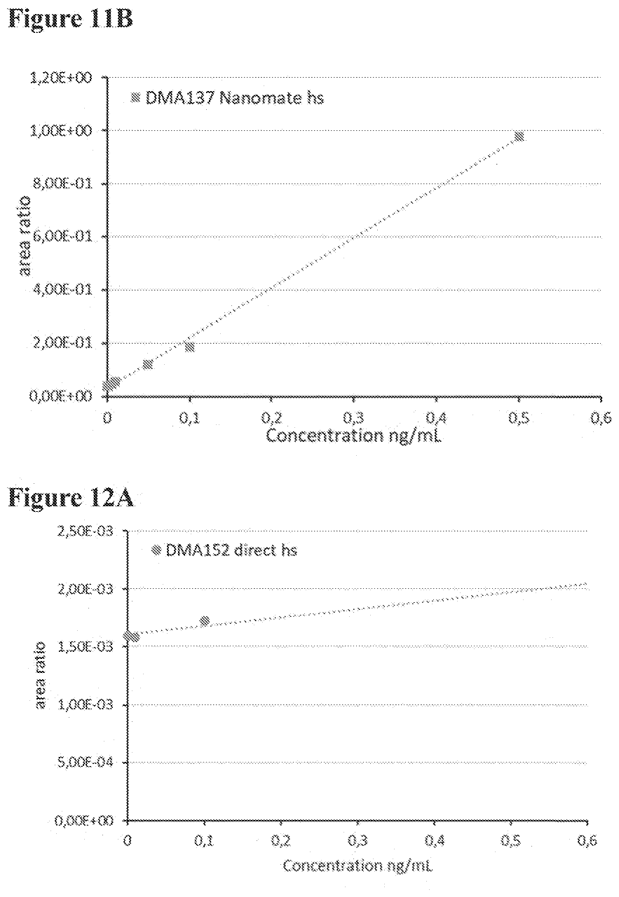

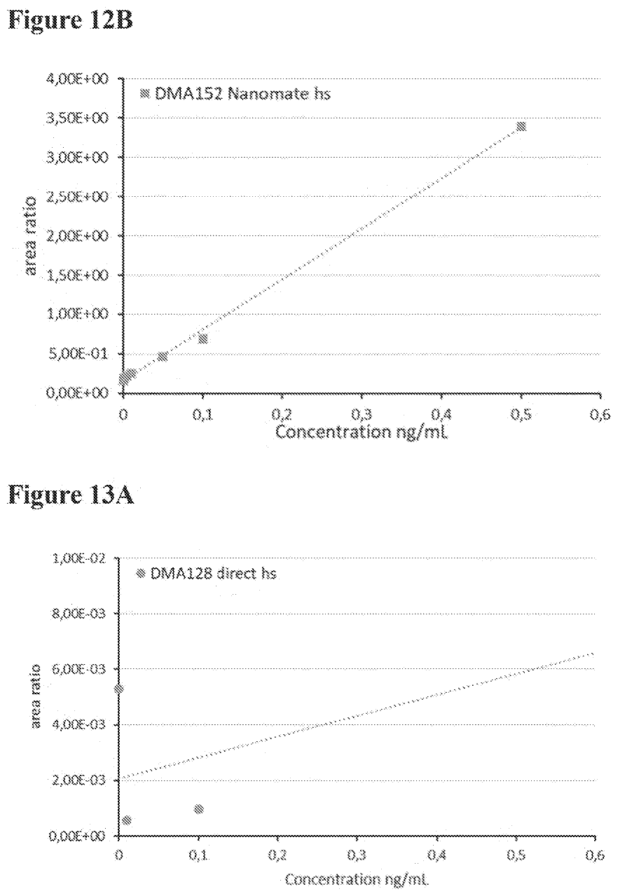

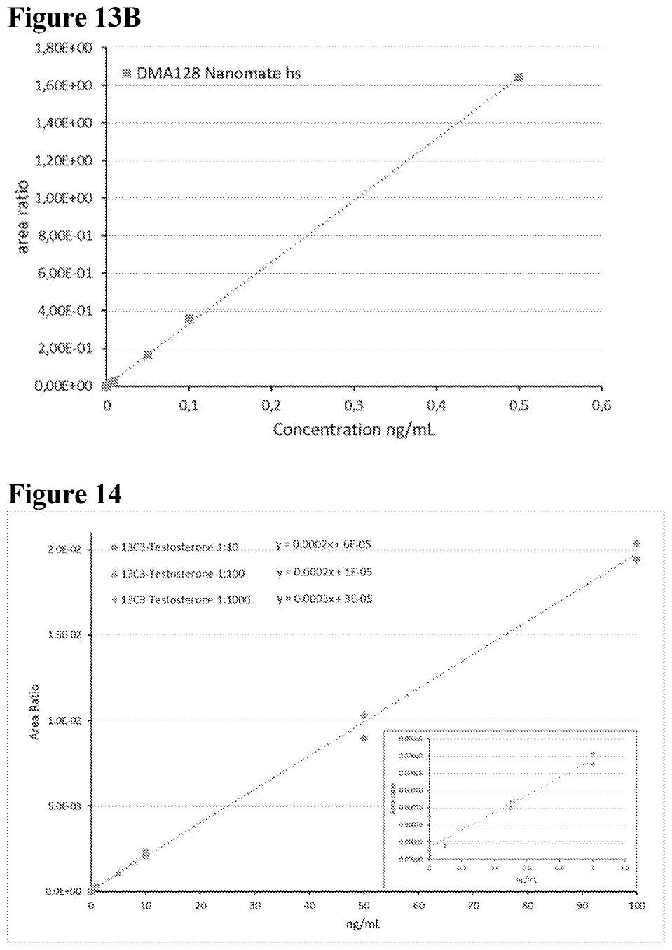

Experimental data support these reduced matrix influences in nano-ESI. FIGS. 8A and 8B show the comparison of nano-ESI and traditional ESI ionization for the analyte Mz2974. It is shown, that the nano-ESI process in FIG. 8B leads to a higher sensitivity compared to the conventional ESI process and high matrix load in FIG. 8A. The same effect of matrix suppression was demonstrated for the analytes DMA098 (FIGS. 9A and 9B), DMA137 (FIGS. 11A and 11B), DMA152 (FIGS. 12A and 12B), and DMA128 (FIGS. 13A and B). In conclusion, a combination of a derivatization process, dilution and the use of nano-ESI leads to increased signal intensities. In embodiments of the first aspect of the invention, the amount or concentration or level of the analyte, in particular the relative amount of the analyte in the pretreated sample can be determined. The method is highly accurate and gives coefficient of variation (CV) of 20% or less, particularly of 10% or less, more particularly 2% or less, e.g. 1% to 2% when repeatedly determining the amount of the analyte.

According to step (a), the pretreated sample is provided. The pretreated sample is preferably a pretreated sample of bodily fluid including the analyte of interest. The pretreated sample is a sample of bodily fluid comprising the analyte of interest.

In embodiments of the first aspect of the invention, the pretreated sample is obtained from a patient sample, which is selected from a group consisting of serum, plasma and whole blood sample from an individual.

In embodiments of the first aspect of the invention, the pretreated sample is a hemolysed whole-blood sample, particularly a hemolysed human whole-blood sample, e.g. derived from a subject the blood of which to be tested for the amount of the analyte of interest. Hemolysis is particularly carried out by dilution with water (H2O), e.g. deionized or distilled water, in particular in a ratio of sample: water of about 1:2 to about 1:20, in particular about 1:5 to about 1:10, in particular about 1:9 (v/v). The sample may be hemolysed for a time less than about 30 min, less than about 10 min, less than about 5 min or even less than about 2 min. In particular embodiments, the sample is hemolyzed for a time of about 10 to about 60 sec.

In particular embodiments, the hemolysis is carried out by mixing sample and water, in particular by vortexing sample and water. In particular sample and water are mixed, in particular vortexed, for about 1 to about 60 sec, in particular for about 5 to about 30 sec. in particular for about 10 sec.

During hemolysis the sample may be kept at a temperature of 20° C. to 30° C., in particular at 22° C. to 25° C., in particular at room temperature.

In particular embodiments, the hemolysis of the sample is carried out by mixing the sample with water in a ratio of 1:9 by vortexing for 10 sec at room temperature.

According to step (a) a pretreated sample comprising internal standard is provided. The internal standard (preferably an isotopically labelled analyte) is dissolved in an appropriate solvent and added to the sample in a defined concentration.

According to step (a) a pretreated sample comprising solid samples for elution is provided. The elution of solid samples is, for example, the elution of dried blood spots. For analysis, the analyte can require elution out of the filter paper along with the blood matrix by using appropriate extractor buffers. Efficient elution of the analyte can demand well-defined extraction parameters (e.g extractor solution, duration, temperature, etc.).

In embodiments of the first aspect of the invention, the pretreated sample is free of a tissue sample or the pretreated sample is not a tissue sample. In particular, the pretreated sample, which is free of a tissue sample is a blood sample, which is contaminated by tissue. In particular, pretreated sample, which is not a tissue sample, does not comprise any tissue.

In embodiments of the first aspect of the invention, the pretreated sample is obtained by at least one or more pre-treatment steps and/or by at least one or more enrichment steps.

In embodiments of the first aspect of the invention, the at least one enrichment step comprises a chemical precipitation or a solid phase, wherein in particular the solid phase is a bead, wherein the bead is magnetic or paramagnetic.

In embodiments of the first aspect of the invention, the chemical precipitation is selected from the following group: acetonitrile, methanol. In general precipitation may occur if the concentration of a compound/analyte exceeds its solubility and/or denaturation.

In embodiments of the first aspect of the invention, the solid phase is a Solid Phase Extraction (SPE) cartridges and/or beads.

In embodiments of the first aspect of the invention, beads are non-magnetic, magnetic, or paramagnetic. Additionally, Beads can be coated differently to be specific for the analyte of interest.

In embodiments of the first aspect of the invention, the coating differs depending on the use intended, i.e. on the intended capture molecule. It is well-known to the skilled person which coating is suitable for which analyte. The beads may be made of various different materials. The beads may have various sizes and comprise a surface with or without pores.

In embodiments of the first aspect of the invention, the method is an in vitro method.

In embodiments of the first aspect of the invention, the method is free of a further step after performing step a) or step b), wherein the further step is selected from the group consisting of extraction step, chromatographic step, lyophilization, centrifuge or combinations thereof.

In embodiments of the first aspect of the invention, the extraction step comprises at least one or more methods selected from the following group: liquid-liquid extraction, liquid-solid extraction, liquid-gas extraction, gas-liquid extraction, solid-liquid extraction, solid phase extraction (SPE).

In embodiments of the first aspect of the invention, the chromatographic step comprises at least one or more methods selected from the following group: chromatography, high performance liquid chromatography (HPLC), liquid chromatography high performance liquid chromatography (LC-HPLC), gel permeation chromatography (GPC), flash chromatography. Chromatography is, for example, size exclusion chromatography.

In embodiments of the first aspect of the invention, the method is automated.

According to step (b), the analyte of interest in the pretreated sample is derivatised.

In embodiments of the first aspect of the invention, step (b) is performed by a compound or label.

In embodiments of the first aspect of the invention, step (b) is performed in a time range of 5 minutes at the maximum, preferably 3 minutes at the maximum, more preferably 2 minutes at the maximum.

In embodiments of the first aspect of the invention, the compound is capable of covalently binding to the analyte or is covalently bounded to the analyte.

In embodiments of the first aspect of the invention, the analyte of interest is derivatized in step b) by a compound, which is capable of forming a covalent binding to the analyte of interest, in particular wherein after step b) the compound is covalently bounded to the analyte of interest for forming a complex with the analyte of interest. A complex of the analyte and compound is formed.

In embodiments of the first aspect of the invention, the compound is simple permanent positively charged or simple permanent negatively charged.

In embodiments of the first aspect of the invention, the compound is double permanent positively charged or double permanent negatively charged.

In embodiments of the first aspect of the invention, the compound comprises more than two permanent positively charged, e.g. 3, 4, 5, 6 or 7, or more than two permanent negatively charges, e.g. 3, 4, 5, 6 or 7.

In embodiments of the first aspect of the invention, the compound is free of a permanent charge.

In embodiments of the first aspect of the invention, the compound has a net charge z1, in particular before fragmentation. After fragmentation the compound can be splitted or cleaved into at least one daughter ion. The daughter ion has a net charge z2, which is smaller than the net charge z1 (z2<z1). A complex comprising or consisting of the analyte and the compound has a net charge z3, in particular before fragmentation. After fragmentation, the complex can be splitted or cleaved into at least one daughter ion having a net charge z4, which is smaller than the net charge z3 (z4<z3). At least one daughter ion can mean in this context that one daughter ion or more are formed after fragmentation. The one daughter ion and the other daughter ions differentiate from each other at least by their mass, charge or structure.

In embodiments of the first aspect of the invention, the compound comprises a permanent charge, in particular a permanent net charge, wherein said compound is capable of covalently binding to the analyte of interest,
wherein said compound has a mass m1 and a net charge z1,
wherein the compound is capable of forming at least one daughter ion having a mass m2<m1 and a net charge z2<z1 after fragmentation by mass spectrometric determination,
wherein m1/z1<m2/z2.

In embodiments of the first aspect of the present invention, the compound is selected from the following group:

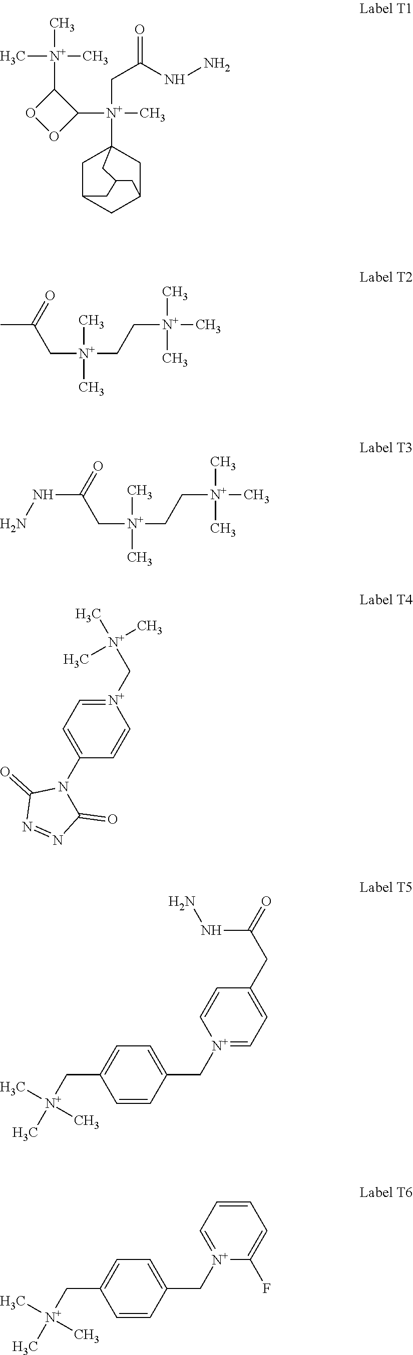

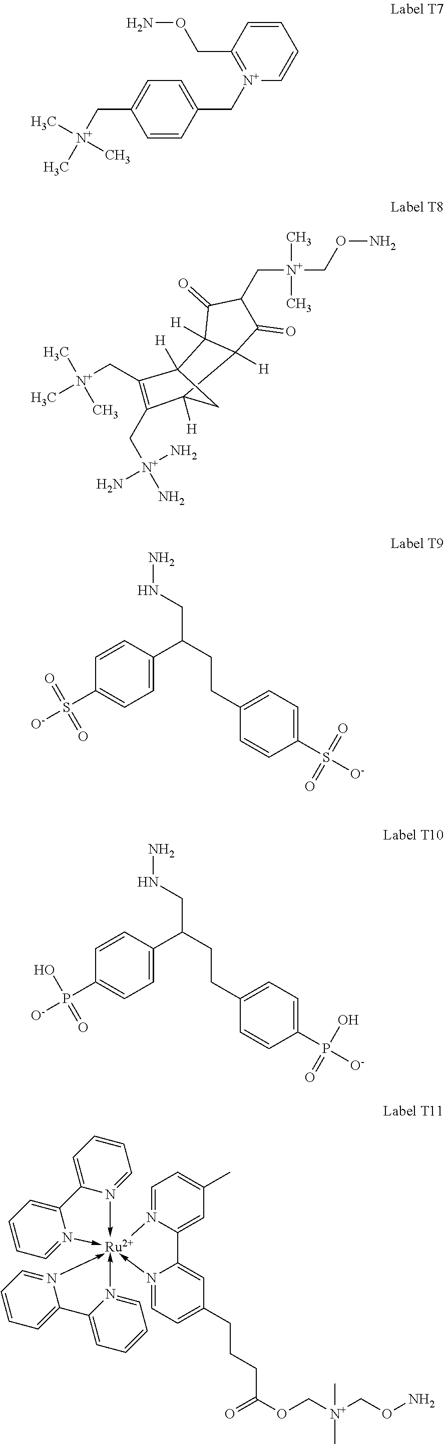

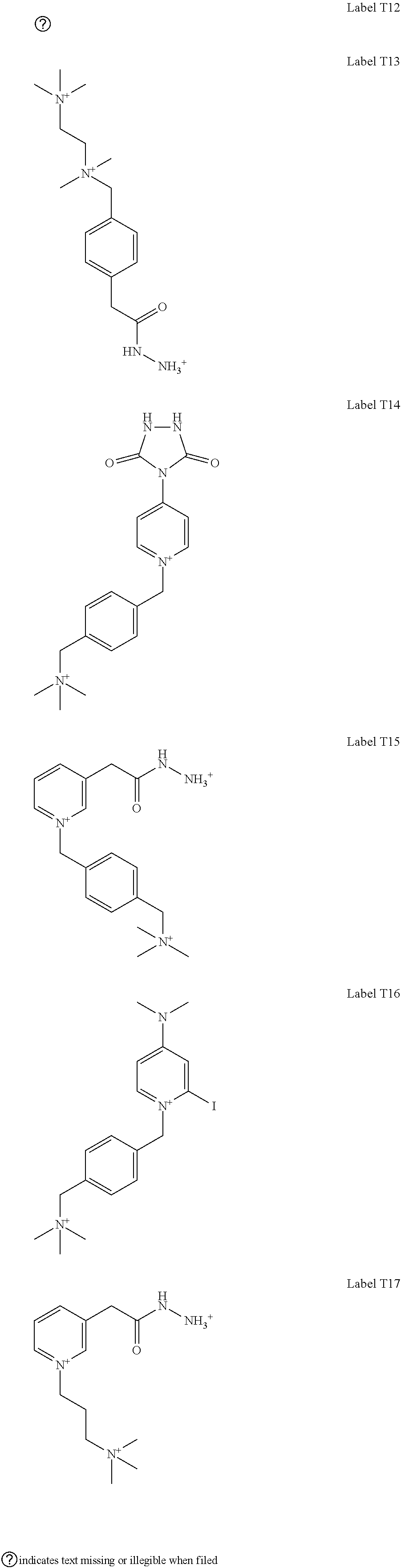 [SMILES: C[N+](C)(C)CC[N+](C)(C)Cc1ccc(CC(=O)N[NH3+])cc1 ; C[N+](C)(C)Cc1ccc(C[n+]2ccc(-n3c(=O)[nH][nH]c3=O)cc2)cc1 ; C[N+](C)(C)Cc1ccc(C[n+]2cccc(CC(=O)N[NH3+])c2)cc1 ; CN(C)c1cc[n+](Cc2ccc(C[N+](C)(C)C)cc2)c(I)c1 ; C[N+](C)(C)CCC[n+]1cccc(CC(=O)N[NH3+])c1]

In embodiments of the first aspect of the invention, the compound comprises a reactive unit K, which is able of reacting with a carbonyl group, phenol group, amine, hydroxyl group or diene group of the analyte of interest.

In embodiments of the first aspect of the invention, K is selected from the group consisting of hydrazide, hydrazine, hydroxylamine, Br, F-aromatic, 4-substituted 1,2,4-triazolin-3,5-dione (TAD), active ester, sulfonylchloride and reactive carbonyl.

In embodiments of the first aspect of the invention, the compound comprises a counter ion for forming a salt, wherein the counter ion is preferably selected from the following group: Cl⁻, Br⁻, F⁻, formiate, trifluoroacetate, PFt₆⁻, sulfonate, phosphate, acetate.

In embodiments of the first aspect of the invention, step b) is performed at a temperature, which is at least 20° C. or more.

In embodiments of the first aspect of the invention, step b) is performed at least at 30° C., for example 35° C.

In embodiments of the first aspect of the invention, step b) is performed at least at 40° C., for example 45° C.

In embodiments of the first aspect of the invention, step b) is performed at least at 50° C., for example 55° C.

In embodiments of the first aspect of the invention, step b) is performed at least at 60° C. for example 65° C.

In embodiments of the first aspect of the invention, step b) is performed at least at 70° C., for example 75 ¹³C.

In embodiments of the first aspect of the invention, step b) is performed at least at 80° C., for example 85° C.

In embodiments of the first aspect of the invention, step b) comprises the addition of a further substance or further substances. Theses further substance or further substances are, e.g. additives. The further substance or the further substances are, for example, for protonation and/or for catalysis. In particular the further substance or the further substances for catalysis is or are (a) lewis base(s).

In embodiments of the first aspect of the invention, a further substance or further substances for protonation are selected from the group consisting of protonating organic acids. e.g. formic acid.

In embodiments of the first aspect of the invention, a further substance or further substances for catalysis are selected from the group consisting of lewis bases, e.g. phenylenediamine.

In embodiments of the first aspect of the invention, the method comprises the compound of formula A or B:

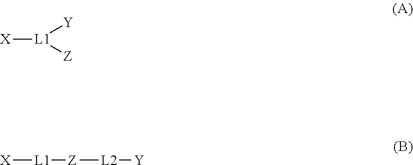

wherein
X is a reactive unit, which is in particular capable of forming a covalent bond with an analyte of interest,
L1 and L2 are independently of each other substituted or unsubstituted
linker, in particular branched or linear linker,
Y is a neutral loss unit, and
Z is a charged unit comprising at least one permanently charged moiety, in particular comprising one permanently charged moiety,
including any salt thereof, and/or
comprising the compound of formula P1:

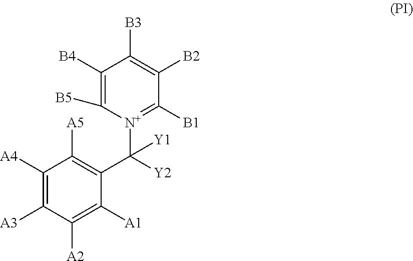

wherein one of the substituents B1, B2, B3, B4, B5 is a coupling group Q, which is capable of forming a covalent bond with the analyte,
wherein the other substituents A1, A2, A3, A4, A5, B1, B2, B3, B4, B5 are each independently selected from hydrogen, halogen, alkyl, N-acylamino, N,N-dialkylamino, alkoxy, thioalkoxy, hydroxy, cyano, alkoxycarbonyl, alkoxythiocarbonyl, acyl, nitro, thioacyl, aryloyl, fluoromethyl, difluoromethyl, trifluoromethyl, trifluoroethyl, cyanomethyl, cyanoethyl, hydroxyethyl, methoxyethyl, nitroethyl, acyloxy, aryloyloxy, cycloalkyl, aryl, heteroaryl, heterocycloalkyl, amino, isotope or derivative thereof,
wherein Y1 and Y2 are each independently selected from hydrogen, methyl, ethyl, methoxy, substituted aromatic, unsubstituted aromatic, substituted cycloalkyl, unsubstituted cycloalkyl, substituted heteroaromatic, unsubstituted heteroaromatic, amine or wherein Y1 and Y2 form a ring structure, which is selected from substituted cycloalkyl, unsubstituted cycloalkyl, substituted aromatic, unsubstituted aromatic, substituted heteroaromatic, unsubstituted heteroaromatic, and/or
comprising the compound of formula D1:

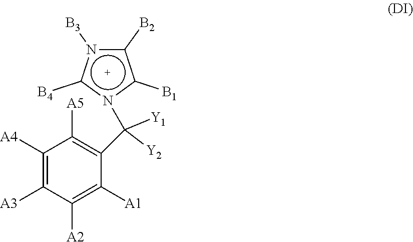

wherein one of the substituents B1, B2, B4 is a coupling group Q, which is capable of forming a covalent bond with the analyte,
wherein the other substituents A1, A2, A3, A4. A5, B1, B2, B4 are each independently selected from hydrogen, halogen, alkyl, N-acylamino. N,N-dialkylamino, alkoxy, thioalkoxy, hydroxy, cyano, alkoxycarbonyl, alkoxythiocarbonyl, acyl, nitro, thioacyl, aryloyl, fluoromethyl, difluoromethyl, trifluoromethyl, trifluoroethyl, cyanomethyl, cyanoethyl, hydroxyethyl, methoxyethyl, nitroethyl, acyloxy, aryloyloxy, cycloalkyl, aryl, heteroaryl, heterocycloalkyl, amino, isotope or derivative thereof,
wherein B3 is selected from alkyl, acetyl, vinyl, substituted aromatic, unsubstituted aromatic, substituted benzyl, unsubstituted benzyl, substituted cycloalkyl, unsubstituted cycloalkyl, isotope and derivative thereof,
wherein Y1 and Y2 are each independently selected from hydrogen, methyl, ethyl, methoxy, substituted aromatic, unsubstituted aromatic, substituted cycloalkyl, unsubstituted cycloalkyl, substituted heteroaromatic, unsubstituted heteroaromatic, amine or wherein Y1 and Y2 form a ring structure, which is selected from substituted cycloalkyl, unsubstituted cycloalkyl, substituted aromatic, unsubstituted aromatic, substituted heteroaromatic, unsubstituted heteroaromatic, and/or comprising the compound of formula C1:

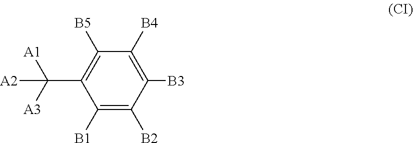

wherein one of the substituents B1, B2, B3, B4, B5 is a coupling group Q, which is capable of forming a covalent bond with the analyte,
wherein the other substituents A 1, A2, B1, B2, B3, B4, B5 are each independently selected from hydrogen, halogen, alkyl, modified alkyl, N-acylamino, N,N-dialkylamino, alkoxy, thioalkoxy, hydroxy, cyano, alkoxycarbonyl, alkoxythiocarbonyl, acyl, nitro, thioacyl, aryloyl, fluoromethyl, difluoromethyl, trifluoromethyl, trifluoroethyl, cyanomethyl, cyanoethyl, hydroxyethyl, methoxyethyl, nitroethyl, acyloxy, aryloyloxy, cycloalkyl, aryl, heteroaryl, heterocycloalkyl, amino, sulfur, isotope or derivative thereof,
wherein A3 comprises ammonium, pyridinium, phosphonium or derivatives thereof,
wherein in case of A3 is ammonium and B1 or B5 is the coupling group Q, the coupling group Q comprises a C atom, which is separated by four single or double bonds from the C atom of the CA1A2A3 substituent and the coupling group Q comprises a C-atom, which is separated by five single or double bonds from the C atom of the CA1A2A3 substituent.

In embodiments of the first aspect of the invention, the compound comprises formula A or B:

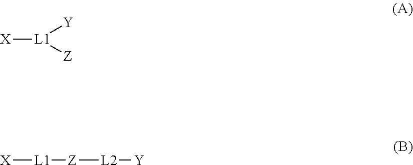

wherein
X is a reactive unit, which is in particular capable of forming a covalent bond with an analyte of interest,
L1 and L2 are independently of each other substituted or unsubstituted
linker, in particular branched or linear linker,
Y is a neutral loss unit, and
Z is a charged unit comprising at least one permanently charged moiety, in particular comprising one permanently charged moiety,
including any salt thereof.

In embodiments of the first aspect of the present invention, the compound of formula A is selected from the group consisting of

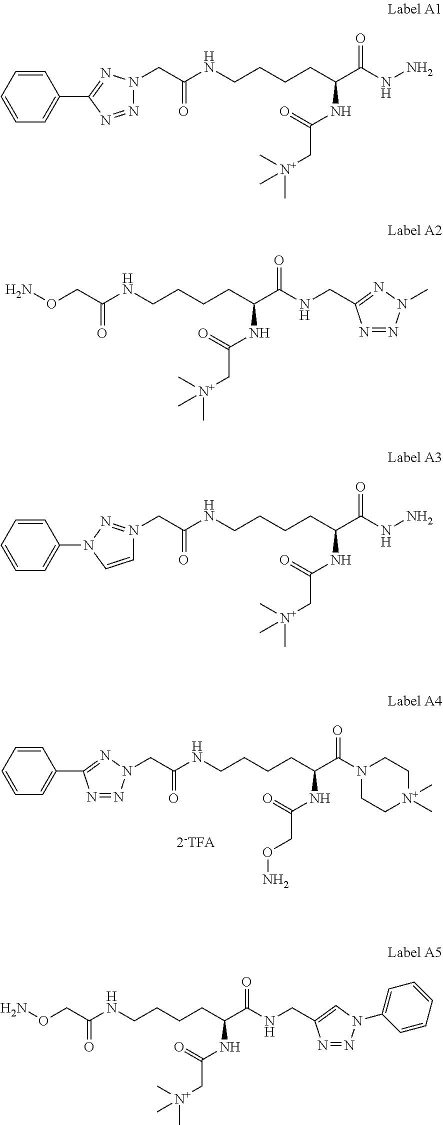

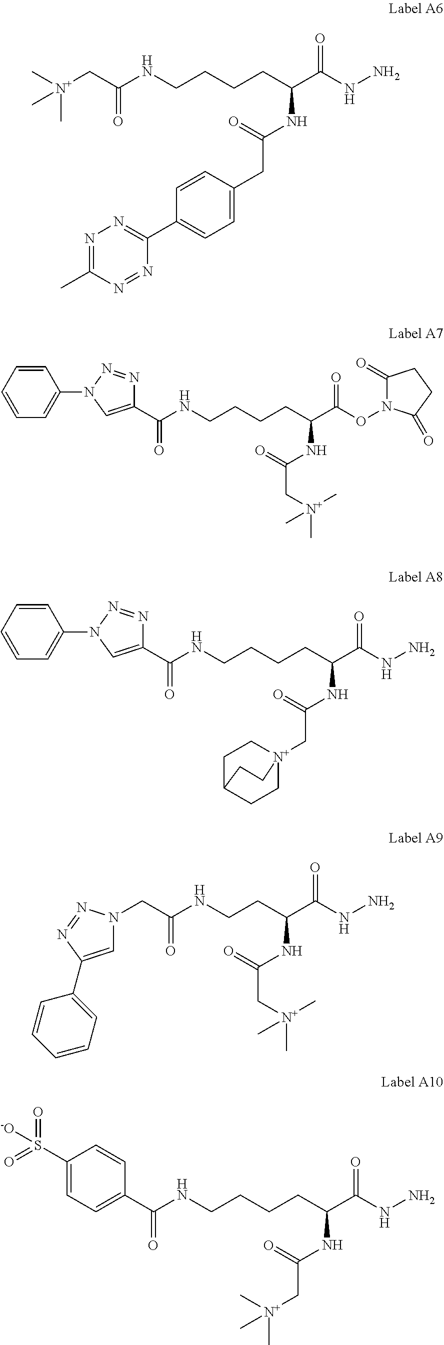

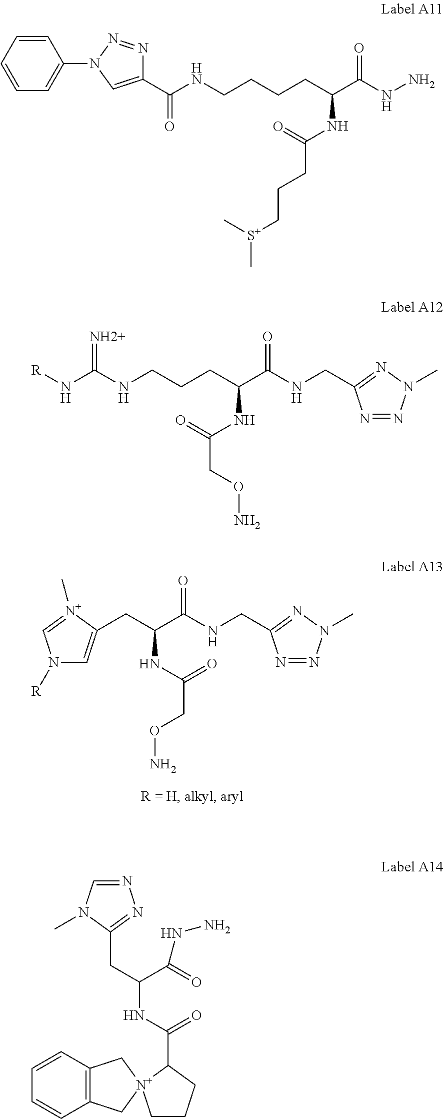

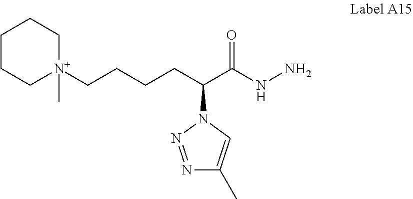 [SMILES: Cc1cn([C@@H](CCCC[N+]2(C)CCCCC2)C(=O)NN)nn1]

or combinations thereof.

In embodiments of the first aspect of the present invention, the compound of formula B is selected from the group consisting of

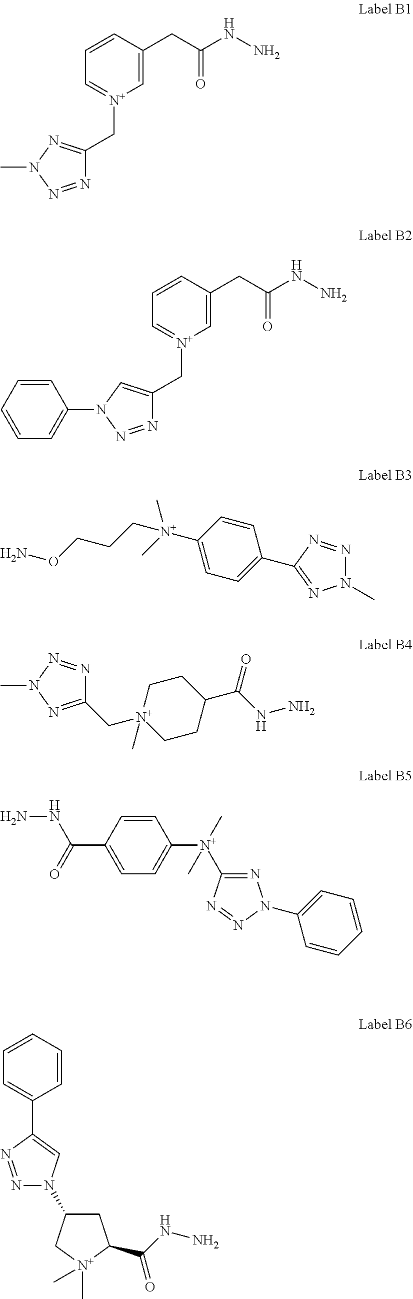

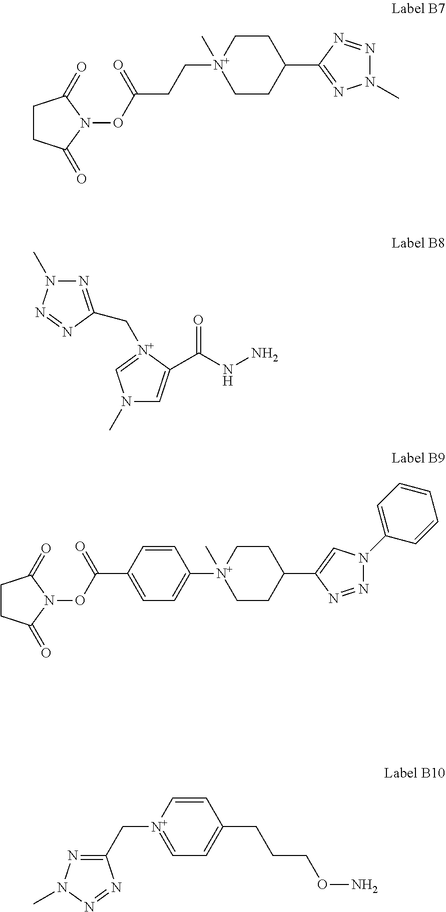

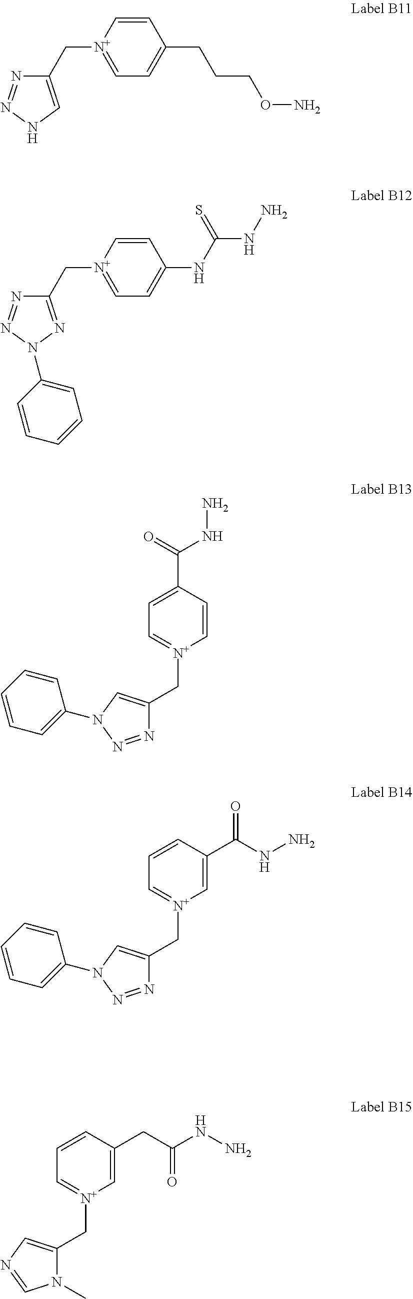 [SMILES: NOCCCc1cc[n+](Cc2c[nH]nn2)cc1 ; NNC(=S)Nc1cc[n+](Cc2nnn(-c3ccccc3)n2)cc1 ; NNC(=O)c1cc[n+](Cc2cn(-c3ccccc3)nn2)cc1 ; NNC(=O)c1ccc[n+](Cc2cn(-c3ccccc3)nn2)c1 ; Cn1cncc1C[n+]1cccc(CC(=O)NN)c1]

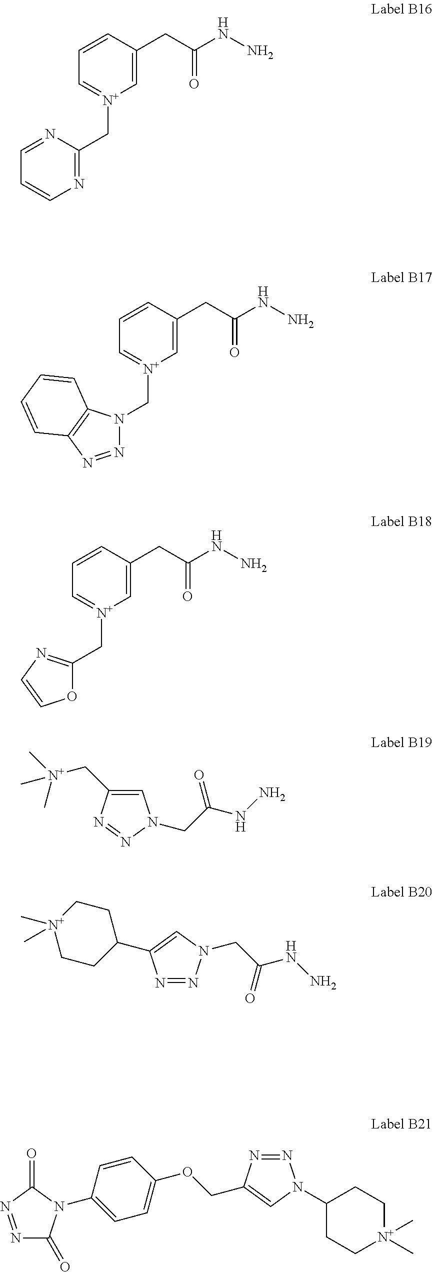 [SMILES: NNC(=O)Cc1ccc[n+](Cc2ncccn2)c1 ; NNC(=O)Cc1ccc[n+](Cn2nnc3ccccc32)c1 ; NNC(=O)Cc1ccc[n+](Cc2ncco2)c1 ; C[N+](C)(C)Cc1cn(CC(=O)NN)nn1 ; C[N+]1(C)CCC(c2cn(CC(=O)NN)nn2)CC1 ; C[N+]1(C)CCC(n2cc(COc3ccc(N4C(=O)N=NC4=O)cc3)nn2)CC1]

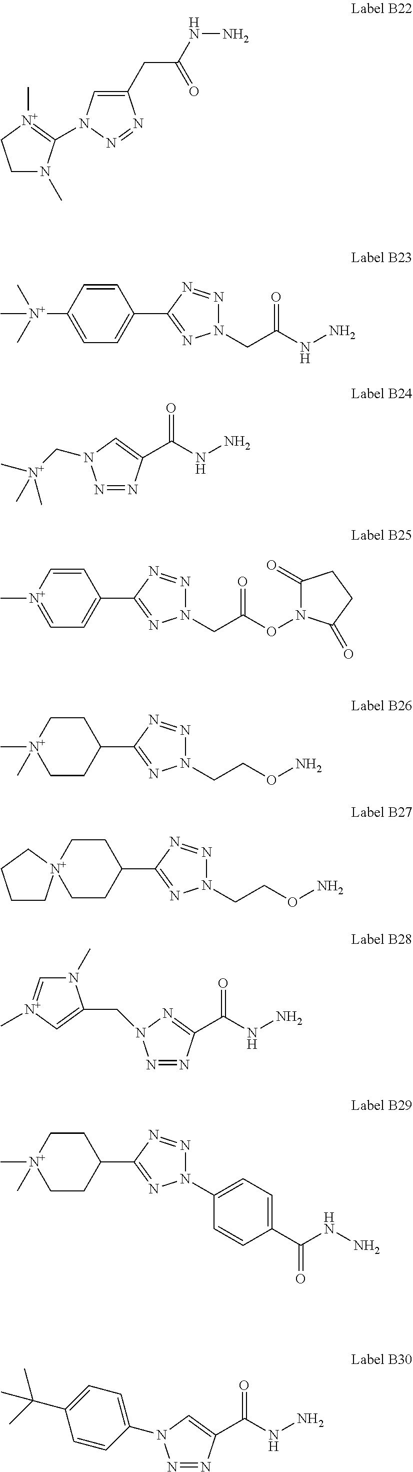

or combinations thereof.

In embodiments of the first aspect of the invention, the compound is selected from the group consisting of: dansylchloride, carbamic acid, N-[2-[[[2-(diethylamino)ethyl]amino]carbonyl]-6-quinolinyl]-, 2,5-dioxo-1-pyrrolidinyl ester (RapiFluor-MS), 4-substituted 1,2,4-triazoline-3,5-diones (Cookson-type reagents), 4-Phenyl-1,2,4-triazolin-3,5-dion-derivative (Amplifex Diene). I-propanaminium, 3-(aminooxy)-N,N,N-trimethyl-compound comprising an appropriate counter ion, e.g. bromide, chloride, iodine, etc. (Amplifex Keto), acethydrazide trimethylammonium chloride (Girard T), 1-(carboxymethyl)pyridinium chloride hydrazide (Girard P) and pyridiyl amine.

In embodiments of the first aspect of the invention, at least one possible chemical structure of the compound is:

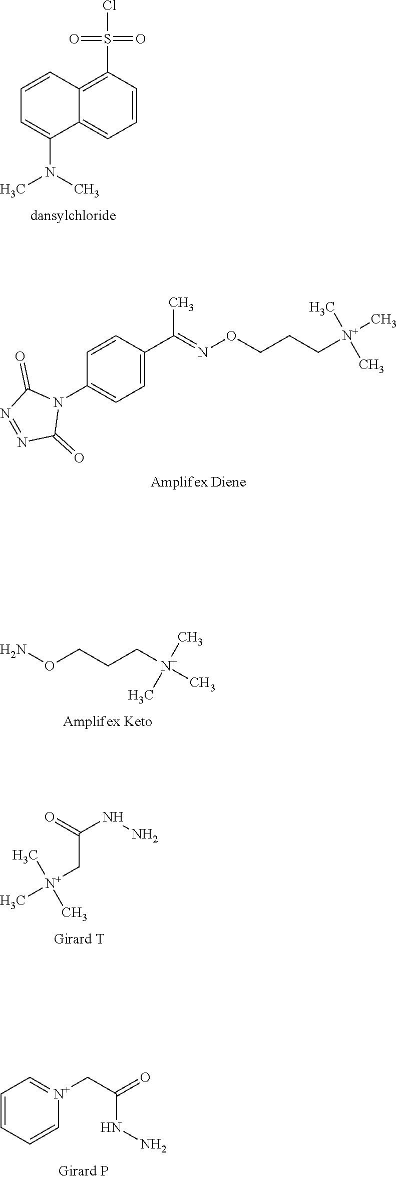

In embodiments of the first aspect of the invention, the method comprises the compound of formula P1:

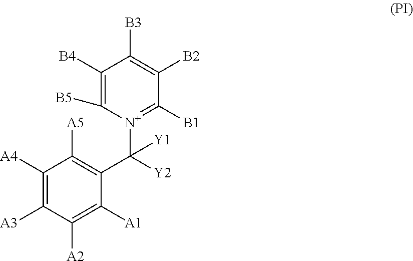

wherein one of the substituents B1, B2, B3, B4, B5 is a coupling group Q, which is capable of forming a covalent bond with the analyte,
wherein the other substituents A1, A2, A3, A4, A5, B1, B2, B3, B4, B5 are each independently selected from hydrogen, halogen, alkyl, N-acylamino. N,N-dialkylamino, alkoxy, thioalkoxy, hydroxy, cyano, alkoxycarbonyl, alkoxythiocarbonyl, acyl, nitro, thioacyl, aryloyl, fluoromethyl, difluoromethyl, trifluoromethyl, trifluoroethyl, cyanomethyl, cyanoethyl, hydroxyethyl, methoxyethyl, nitroethyl, acyloxy, aryloyloxy, cycloalkyl, aryl, heteroaryl, heterocycloalkyl, amino, isotope or derivative thereof,
wherein Y1 and Y2 are each independently selected from hydrogen, methyl, ethyl, methoxy, substituted aromatic, unsubstituted aromatic, substituted cycloalkyl, unsubstituted cycloalkyl, substituted heteroaromatic, unsubstituted heteroaromatic, amine or wherein Y1 and Y2 form a ring structure, which is selected from substituted cycloalkyl, unsubstituted cycloalkyl, substituted aromatic, unsubstituted aromatic, substituted heteroaromatic, unsubstituted heteroaromatic.

In embodiments of the first aspect of the present invention, the compound of formula P1 is selected from the following group:

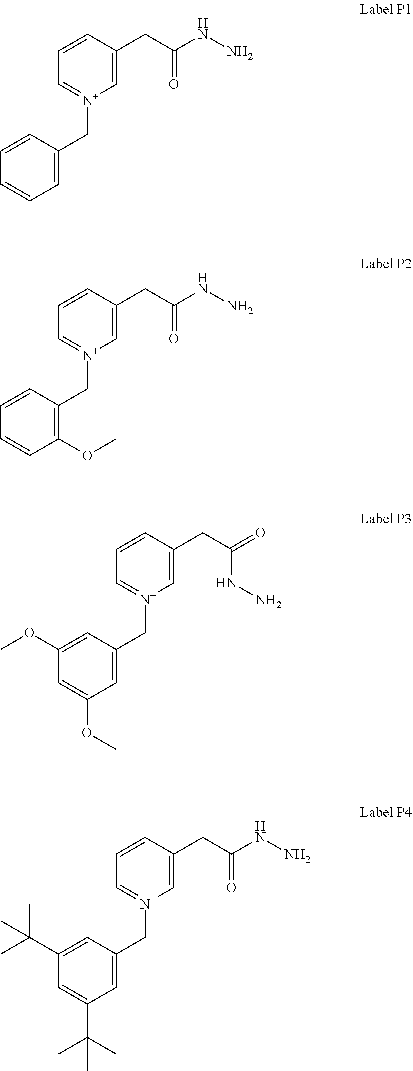 [SMILES: NNC(=O)Cc1ccc[n+](Cc2ccccc2)c1 ; COc1ccccc1C[n+]1cccc(CC(=O)NN)c1 ; COc1cc(C[n+]2cccc(CC(=O)NN)c2)cc(OC)c1 ; CC(C)(C)c1cc(C[n+]2cccc(CC(=O)NN)c2)cc(C(C)(C)C)c1]

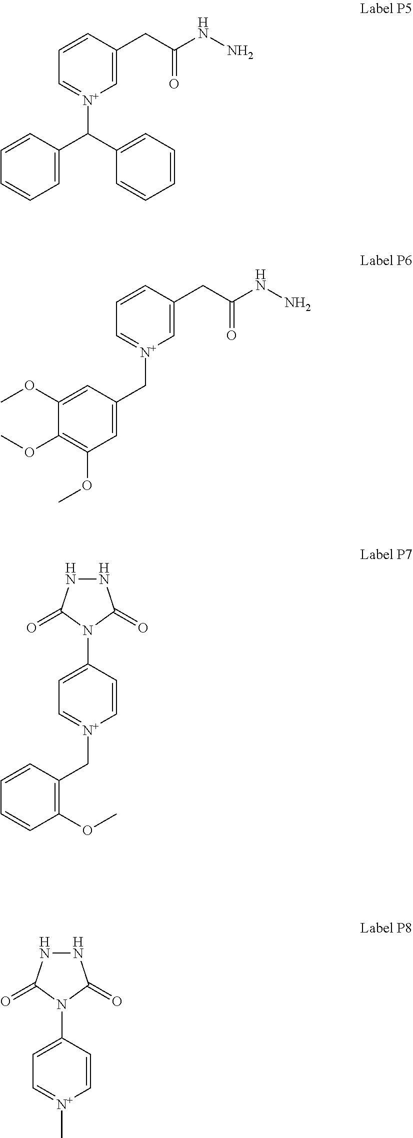 [SMILES: NNC(=O)Cc1ccc[n+](C(c2ccccc2)c2ccccc2)c1 ; COc1cc(C[n+]2cccc(CC(=O)NN)c2)cc(OC)c1OC ; COc1ccccc1C[n+]1ccc(-n2c(=O)[nH][nH]c2=O)cc1 ; C[n+]1ccc(-n2c(=O)[nH][nH]c2=O)cc1]

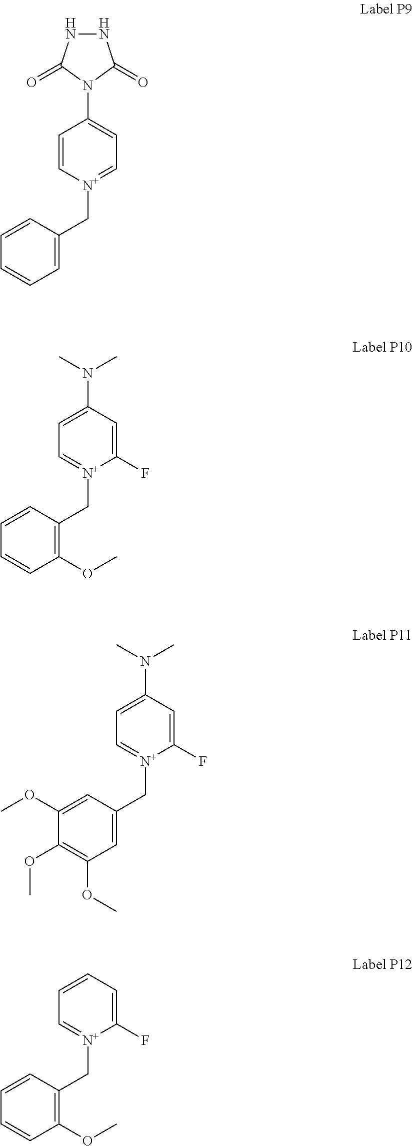 [SMILES: O=c1[nH][nH]c(=O)n1-c1cc[n+](Cc2ccccc2)cc1 ; COc1ccccc1C[n+]1ccc(N(C)C)cc1F ; COc1cc(C[n+]2ccc(N(C)C)cc2F)cc(OC)c1OC ; COc1ccccc1C[n+]1ccccc1F]

or combinations thereof.

In embodiments of the first aspect of the invention, the method comprises the compound of formula D1:

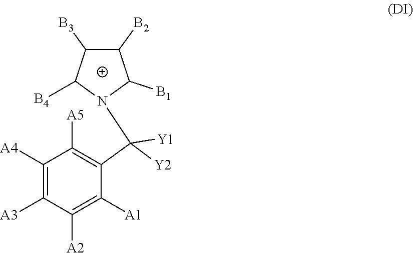

wherein one of the substituents B1, B2, B4 is a coupling group Q, which is capable of forming a covalent bond with the analyte,
wherein the other substituents A1, A2, A3, A4, A5, B1, B2, B4 are each independently selected from hydrogen, halogen, alkyl. N-acylamino, N, N-dialkylamino, alkoxy, thioalkoxy, hydroxy, cyano, alkoxycarbonyl, alkoxythiocarbonyl, acyl, nitro, thioacyl, aryloyl, fluoromethyl, difluoromethyl, trifluoromethyl, trifluoroethyl, cyanomethyl, cyanoethyl, hydroxyethyl, methoxyethyl, nitroethyl, acyloxy, aryloyloxy, cycloalkyl, aryl, heteroaryl, heterocycloalkyl, amino, isotope or derivative thereof,
wherein B3 is selected from alkyl, acetyl, vinyl, substituted aromatic, unsubstituted aromatic, substituted benzyl, unsubstituted benzyl, substituted cycloalkyl, unsubstituted cycloalkyl, isotope and derivative thereof,
wherein Y1 and Y2 are each independently selected from hydrogen, methyl, ethyl, methoxy, substituted aromatic, unsubstituted aromatic, substituted cycloalkyl, unsubstituted cycloalkyl, substituted heteroaromatic, unsubstituted heteroaromatic, amine or wherein Y1 and Y2 form a ring structure, which is selected from substituted cycloalkyl, unsubstituted cycloalkyl, substituted aromatic, unsubstituted aromatic, substituted heteroaromatic, unsubstituted heteroaromatic.

In embodiments of the first aspect of the present invention, the compound of formula D1 is selected from the following group:

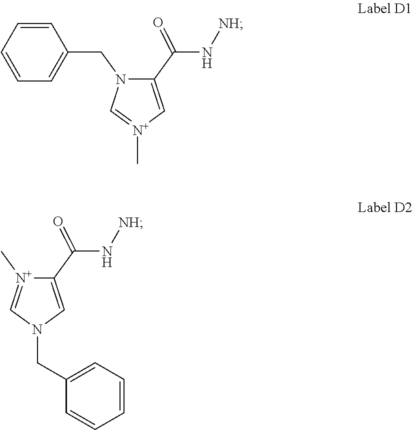

or combinations thereof.

In embodiments of the first aspect of the present invention, the method comprises compound of formula C1:

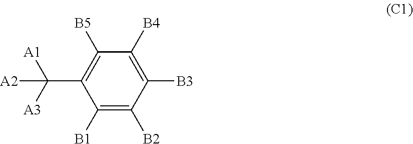

wherein one of the substituents B1, B2. B3, B4. B5 is a coupling group Q, which is capable of forming a covalent bond with the analyte,
wherein the other substituents A1, A2, B1, B2, B3, B4, B5 are each independently selected from hydrogen, halogen, alkyl, modified alkyl, N-acylamino, N,N-dialkylamino, alkoxy, thioalkoxy, hydroxy, cyano, alkoxycarbonyl, alkoxythiocarbonyl, acyl, nitro, thioacyl, aryloyl, fluoromethyl, difluoromethyl, trifluoromethyl, trifluoroethyl, cyanomethyl, cyanoethyl, hydroxyethyl, methoxyethyl, nitroethyl, acyloxy, aryloyloxy, cycloalkyl, aryl, heteroaryl, heterocycloalkyl, amino, sulfur, isotope or derivative thereof,
wherein A3 comprises ammonium, pyridium, phosphonium or derivatives thereof,
wherein in case of A3 is ammonium and B1 or B5 is the coupling group Q, the coupling group Q comprises a C atom, which is separated by four single or double bonds from the C atom of the CA A2A3 substituent and the coupling group Q comprises a C-atom, which is separated by five single or double bonds from the C atom of the CA1A2A3 substituent.

In embodiments of the first aspect of the present invention, the compound of formula C1 is selected from the follow ng group:

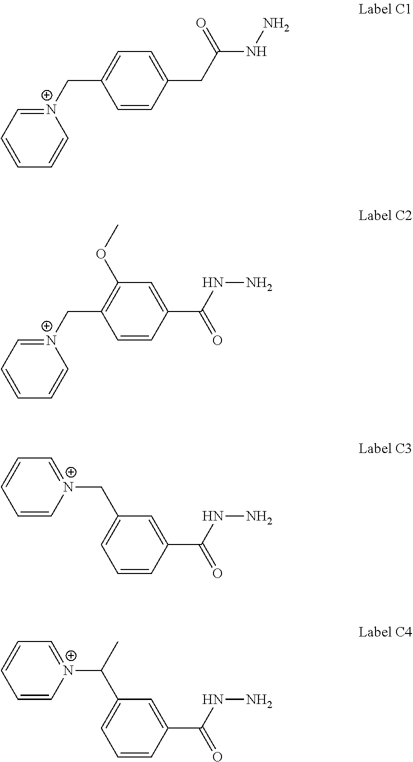 [SMILES: NNC(=O)Cc1ccc(C[n+]2ccccc2)cc1 ; COc1cc(C(=O)NN)ccc1C[n+]1ccccc1 ; NNC(=O)c1cccc(C[n+]2ccccc2)c1 ; CC(c1cccc(C(=O)NN)c1)[n+]1ccccc1]

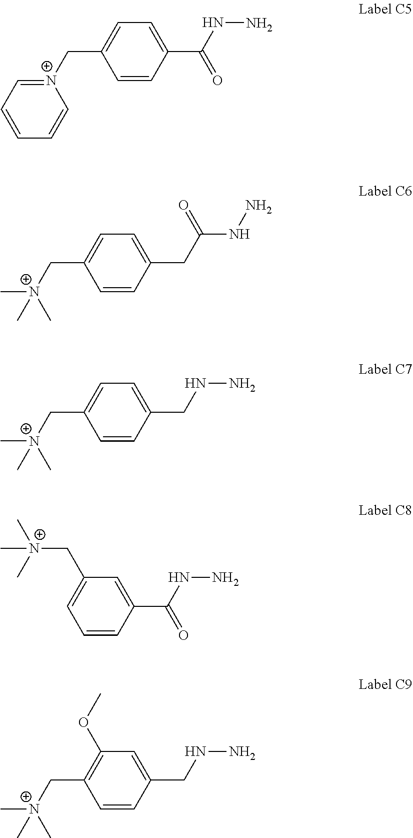 [SMILES: NNC(=O)c1ccc(C[n+]2ccccc2)cc1 ; C[N+](C)(C)Cc1ccc(CC(=O)NN)cc1 ; C[N+](C)(C)Cc1ccc(CNN)cc1 ; C[N+](C)(C)Cc1cccc(C(=O)NN)c1 ; COc1cc(CNN)ccc1C[N+](C)(C)C]

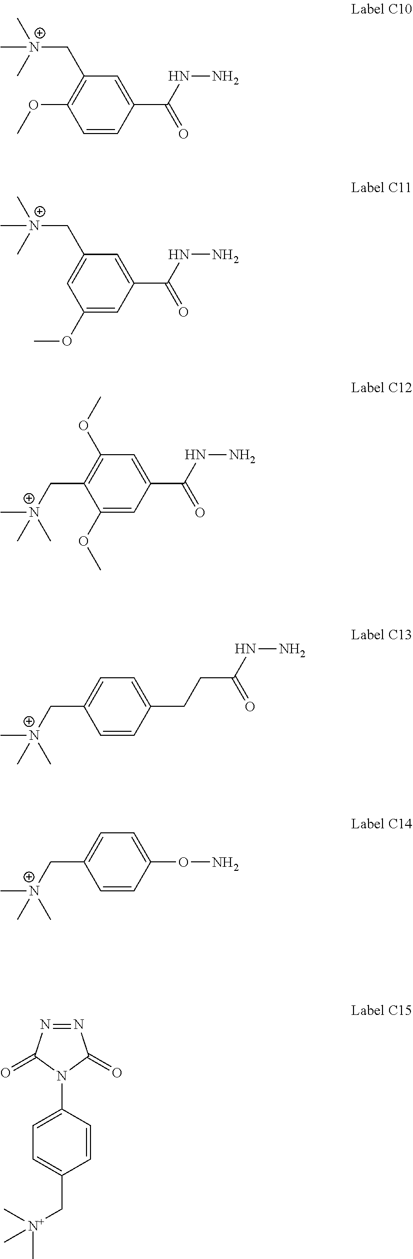 [SMILES: COc1ccc(C(=O)NN)cc1C[N+](C)(C)C ; COc1cc(C[N+](C)(C)C)cc(C(=O)NN)c1 ; COc1cc(C(=O)NN)cc(OC)c1C[N+](C)(C)C ; C[N+](C)(C)Cc1ccc(CCC(=O)NN)cc1 ; C[N+](C)(C)Cc1ccc(ON)cc1 ; C[N+](C)(C)Cc1ccc(N2C(=O)N=NC2=O)cc1]

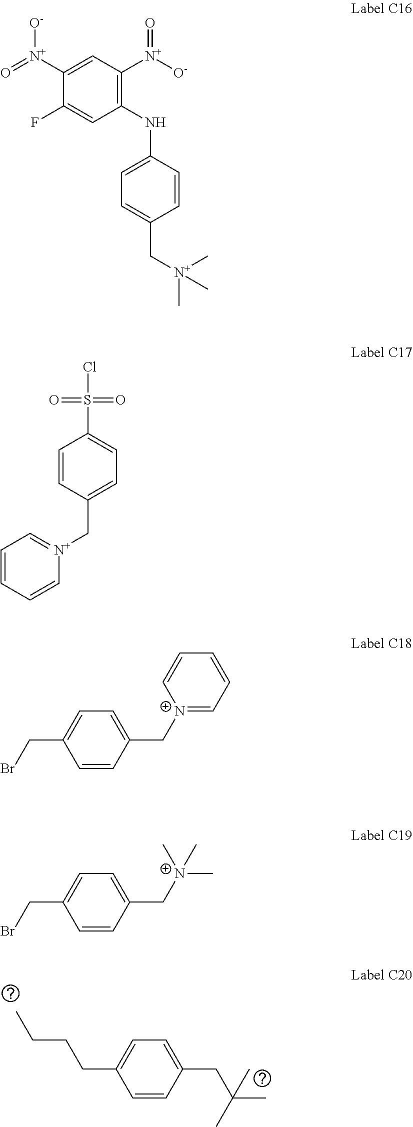

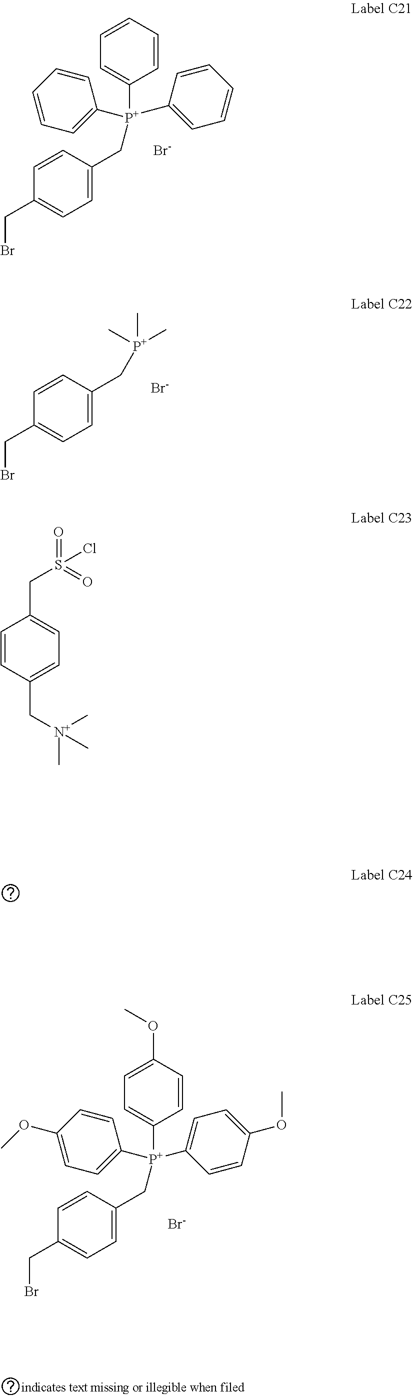

or combinations thereof.

In embodiments of the first aspect of the invention, the ratio of the analyte of interest to the compound is in the range of 1:1 to 1:6.000.000 in step (b). In particular, the ratio of the analyte of interest to the compound is in the range of 1:50000 to 1:100000 or 1:5000 to 1:10000 or 1:1 to 1:100 or 1:100 to 1:1000 or 1:1000000 to 1:2000000.

The ratio depends on the kind of reaction, compound (derivatisation reagent), reaction kinetics, like reaction velocity, and/or temperature. The compound can be provided in an excess comparted to the analyte.

In embodiments of the first aspect of the invention, the analyte of interest is selected from the group consisting of nucleic acid, amino acid, peptide, protein, metabolite, hormones, fatty acid, lipid, carbohydrate, steroid, ketosteroid, secosteroid, a molecule characteristic of a certain modification of another molecule, a substance that has been internalized by the organism, a metabolite of such a substance and combination thereof.

In embodiments of the first aspect of the present invention, the analyte molecule comprises a functional group selected from the group consisting of carbonyl group, diene group, hydroxyl group, amine group, imine group, ketone group, aldehyde group, thiol group, diol group, phenolic group, expoxid group, disulfide group, nucleobase group, carboxylic acid group, terminal cysteine group, terminal serine group and azide group, each of which is capable of forming a covalent bond with reactive unit K of compound. Further, it is also contemplated within the scope of the present invention that a functional group present on an analyte molecule would be first converted into another group that is more readily available for reaction with reactive unit K of compounds.

In embodiments of the first aspect of the present invention, the analyte molecule comprises a carbonyl group as functional group which is selected from the group consisting of a carboxylic acid group, aldehyde group, keto group, a masked aldehyde, masked keto group, ester group, amide group, and anhydride group. Aldoses (aldehyde and keto) exist as acetal and hemiacetals, a sort of masked form of the parent aldehyde/keto.

In embodiments of the first aspect of the present invention, the carbonyl group is an amide group, the skilled person is well aware that the amide group as such is a stable group, but that it can be hydrolyzed to convert the amide group into an carboxylic acid group and an amino group. Hydrolysis of the amide group may be achieved via acid/base catalysed reaction or by enzymatic process either of which is well-known to the skilled person. In embodiments of the first aspect of the present invention, wherein the carbonyl group is a masked aldehyde group or a masked keto group, the respective group is either a hemiacetal group or acetal group, in particular a cyclic hemiacetal group or acetal group. In embodiments of the first aspect of the present invention, the acetal group, is converted into an aldehyde or keto group before reaction with the compound.

In embodiments of the first aspect of the present invention, the carbonyl group is a keto group. In embodiments of the first aspect of the present invention, the keto group may be transferred into an intermediate imine group before reacting with the reactive unit of compounds. In embodiments of the first aspect of the present invention, the analyte molecule comprising one or more keto groups is a ketosteroid. In particular embodiments of the first aspect of the present invention, the ketosteroid is selected from the group consisting of testosterone, epitestosterone, dihydrotestosterone (DHT), desoxymethyltestosterone (DMT), tetrahydrogestrinone (THG), aldosterone, estrone, 4-hydroxyestrone, 2-methoxyestrone, 2-hydroxyestrone, 16-ketoestradiol, 16-alpha-hydroxyestrone, 2-hydroxyestrone-3-methylether, prednisone, prednisolone, pregnenolone, progesterone, dehydroepiandrosterone (DHEA), 17-hydroxypregnenolone, 17-hydroxyprogesterone, androsterone, epiandrosterone, A4-androstenedione, 11-deoxycortisol, corticosterone, 21-deoxycortisol, 11-deoxycorticosterone, allopregnanolone and aldosterone.

In embodiments of the first aspect of the present invention, the carbonyl group is a carboxyl group. In embodiments of the first aspect of the present invention, the carboxyl group reacts directly with the compound or it is converted into an activated ester group before reaction with the compound. In embodiments of the first aspect of the present invention, the analyte molecule comprising one or more carboxyl groups is selected from the group consisting of Δ8-tetrahydrocannabinolic acid, benzoylecgonin, salicylic acid, 2-hydroxybenzoic acid, gabapentin, pregabalin, valproic acid, vancomycin, methotrexate, mycophenolic acid, montelukast, repaglinide, furosemide, telmisartan, gemfibrozil, diclofenac, ibuprofen, indomethacin, zomepirac, isoxepac and penicillin. In embodiments of the first aspect of the present invention, the analyte molecule comprising one or more carboxyl groups is an amino acid selected from the group consisting of arginine, lysine, aspartic acid, glutamic acid, glutamine, asparagine, histidine, serine, threonine, tyrosine, cysteine, tryptophan, alanine, isoleucine, leucine, methionine, phenyalanine, valine, proline and glycine.

In embodiments of the first aspect of the present invention, the carbonyl group is an aldehyde group. In embodiments of the first aspect of the present invention, the aldehyde group may be transferred into an intermediate imine group before reacting with the reactive unit of compounds. In embodiments of the first aspect of the present invention, the analyte molecule comprising one or more aldehyde groups is selected from the group consisting of pyridoxal, N-acetyl-D-glucosamine, alcaftadine, streptomycin and josamycin.

In embodiments of the first aspect of the present invention, the carbonyl group is an carbonyl ester group. In embodiments of the first aspect of the present invention, the analyte molecule comprising one or more ester groups is selected from the group consisting of cocaine, heroin, Ritalin, aceclofenac, acetylcholine, amcinonide, amiloxate, amylocaine, anileridine, aranidipine artesunate and pethidine.

In embodiments of the first aspect of the present invention, the carbonyl group is an anhydride group. In embodiments of the first aspect of the present invention, the analyte molecule comprising one or more anhydride groups is selected from the group consisting of cantharidin, succinic anhydride, trimellitic anhydride and maleic anhydride.

In embodiments of the first aspect of the present invention, the analyte molecule comprises one or more diene groups, in particular to conjugated diene groups, as functional group. In embodiments of the first aspect of the present invention, the analyte molecule comprising one or more diene groups is a secosteroid. In embodiments, the secosteroid is selected from the group consisting of cholecalciferol (vitamin D3), ergocalciferol (vitamin D2), calcifediol, calcitriol, tachysterol, lumisterol and tacalcitol. In particular, the secosteroid is vitamin D, in particular vitamin D2 or D3 or derivates thereof. In particular embodiments, the secosteroid is selected from the group consisting of vitamin D2, vitamin D3, 25-hydroxyvitamin D2, 25-hydroxyvitamin D3 (calcifediol), 3-epi-25-hydroxyvitamin D2, 3-epi-25-hydroxyvitamin D3, 1,25-dihydroxyvitamin D2, 1,25-dihydroxyvitamin D3 (calcitriol), 24,25-dihydroxyvitamin D2, 24,25-dihydroxyvitamin D3. In embodiments of the first aspect of the present invention, the analyte molecule comprising one or more diene groups is selected from the group consisting of vitamin A, tretinoin, isotretinoin, alitretinoin, natamycin, sirolimus, amphotericin B, nystatin, everolimus, temsirolimus and fidaxomicin.

In embodiments of the first aspect of the present invention, the analyte molecule comprises one or more hydroxyl group as functional group. In embodiments of the first aspect of the present invention, the analyte molecule comprises a single hydroxyl group or two hydroxyl groups. In embodiments wherein more than one hydroxyl group is present, the two hydroxyl groups may be positioned adjacent to each other (1,2-diol) or may be separated by 1, 2 or 3 C atoms (1,3-diol, 1,4-diol, 1.5-diol, respectively). In particular embodiments of the first aspect, the analyte molecule comprises a 1, 2-diol group. In embodiments, wherein only one hydroxyl group is present. said analyte is selected from the group consisting of primary alcohol, secondary alcohol and tertiary alcohol. In embodiments of the first aspect of the present invention, wherein the analyte molecule comprises one or more hydroxyl groups, the analyte is selected from the group consisting of benzyl alcohol, menthol, L-camitine, pyridoxine, metronidazole, isosorbide mononitrate, guaifenesin, clavulanic acid, Miglitol, zalcitabine, isoprenaline, aciclovir, methocarbamol, tramadol, venlafaxine, atropine, clofedanol, alpha-hydroxyalprazolam, alpha-Hydroxytriazolam, lorazepam, oxazepam. Temazepam, ethyl glucuronide, ethylmorphine, morphine, morphine-3-glucuronide, buprenorphine, codeine, dihydrocodeine, p-hydroxypropoxyphene, O-desmethyltramadol, Desmetramadol, dihydroquinidine and quinidine. In embodiments of the first aspect of the present invention, wherein the analyte molecule comprises more than one hydroxyl groups, the analyte is selected from the group consisting of vitamin C, glucosamine, mannitol, tetrahydrobiopterin, cytarabine, azacitidine, ribavirin, floxuridine, Gemcitabine, Streptozotocin, adenosine, Vidarabine, cladribine, estriol, trifluridine, clofarabine, nadolol, zanamivir, lactulose, adenosine monophosphate, idoxuridine, regadenoson, lincomycin, clindamycin, Canagliflozin, tobramycin, netilmicin, kanamycin, ticagrelor, epirubicin, doxorubicin, arbekacin, streptomycin, ouabain, amikacin, neomycin, framycetin, paromomycin, erythromycin, clarithromycin, azithromycin, vindesine, digitoxin, digoxin, metrizamide, acetyldigitoxin, deslanoside, Fludarabine, clofarabine, gemcitabine, cytarabine, capecitabine, vidarabine, and plicamycin.

In embodiments of the first aspect of the present invention, the analyte molecule comprises one or more thiol group (including but not limited to alkyl thiol and aryl thiol groups) as functional group. In embodiments of the first aspect of the present invention, the analyte molecule comprising one or more thiol groups is selected from the group consisting of thiomandelic acid, DL-captopril, DL-thiorphan, N-acetylcysteine, D-penicillamine, glutathione, L-cysteine, zofenoprilat, tiopronin, dimercaprol, succimer.

In embodiments of the first aspect of the present invention, the analyte molecule comprises one or more disulfide group as functional group. In embodiments of the first aspect of the present invention, the analyte molecule comprising one or more disulfide groups is selected from the group consisting of glutathione disulfide dipyrithione, selenium sulfide, disulfiram, lipoic acid. L-cystine, fursultiamine, octreotide, desmopressin, vapreotide, terlipressin, linaclotide and peginesatide. Selenium sulfide can be selenium disulfide. SeS₂, or selenium hexasulfide, Se₂Se₆.

In embodiments of the first aspect of the present invention, the analyte molecule comprises one or more epoxide group as functional group. In embodiments of the first aspect of the present invention, the analyte molecule comprising one or more epoxide groups is selected from the group consisting of Carbamazepine-10,11-epoxide, carfilzomib, furosemide epoxide, fosfomycin, sevelamer hydrochloride, cerulenin, scopolamine, tiotropium, tiotropium bromide, methylscopolamine bromide, eplerenone, mupirocin, natamycin, and troleandomycin.

In embodiments of the first aspect of the present invention, the analyte molecule comprises one or more phenol groups as functional group. In particular embodiments of the first aspect of the present invention, analyte molecules comprising one or more phenol groups are steroids or steroid-like compounds. In embodiments of the first aspect of the present invention, the analyte molecule comprising one or more phenol groups is a steroid or a steroid-like compound having an A-ring which is sp² hybridized and an OH group at the 3-position of the A-ring. In particular embodiments of the first aspect of the present invention, the steroid or steroid-like analyte molecule is selected from the group consisting of estrogen, estrogen-like compounds, estrone (E1), estradiol (E2). 17a-estradiol, 17b-estradiol, estriol (E3), 16-epiestriol, 17-epiestriol, and 16, 17-epiestriol and/or metabolites thereof. In embodiments, the metabolites are selected from the group consisting of estriol, 16-epiestriol (16-epiE3), 17-epiestriol (17-epiE3), 16,17-epiestriol (16,17-epiE3), 16-ketoestradiol (16-ketoE2), 16a-hydroxyestrone (16a-OHE1), 2-methoxyestrone (2-McOE1), 4-methoxyestrone (4-MeOE1), 2-hydroxyestrone-3-methyl ether (3-MeOE1), 2-methoxyestradiol (2-MeOE2), 4-methoxyestradiol (4-MeOE2), 2-hydroxyestrone (2-OHE1), 4-hydroxyestrone (4-OHE1), 2-hydroxyestradiol (2-OHE2), estrone (E1), estrone sulfate (E1s), 17a-estradiol (E2a), 17b-estradiol (E2B), estradiol sulfate (E2S), equilin (EQ), 17a-dihydroequilin (EQa), 17b-dihydroequilin (EQb), Equilenin (EN), 17-dihydroequilenin (ENa), 17α-dihydroequilenin, 17β-dihydroequilenin (ENb), Δ8,9-dehydroestrone (dE1), Δ8,9-dehydroestrone sulfate (dE1s), Δ9-tetrahydrocannabinol, mycophenolic acid. β or b can be used interchangeable, α and a can be used interchangeable.

In embodiments of the first aspect of the present invention, the analyte molecule comprises an amine group as functional group. In embodiments of the first aspect of the present invention, the amine group is an alkyl amine or an aryl amine group. In embodiments of the first aspect of the present invention, the analyte comprising one or more amine groups is selected from the group consisting of proteins and peptides. In embodiments of the first aspect of the present invention, the analyte molecule comprising an amine group is selected from the group consisting of 3,4-methylenedioxyamphetamine, 3,4-methylenedioxy-N-ethylamphetamine, 3,4-methylenedioxymethamphetamine, Amphetamine. Methamphetamine, N-methyl-1,3-benzodioxolylbutanamine, 7-aminoclonazepam, 7-aminoflunitrazepam, 3,4-dimethylmethcathinone, 3-fluoromethcathinone, 4-methoxymethcathinone, 4-methylethcathinone, 4-methylmethcathinone, amfepramone, butylone, ethcathinone, elephedrone, methcathinone, methylone, methylenedioxypyrovalerone, benzoylecgonine, dehydronorketamine, ketamine, norketamine, methadone, normethadone, 6-acetylmorphine, diacetylmorphine, morphine, norhydrocodone, oxycodone, oxymorphone, phencyclidine, norpropoxyphene, amitriptyline, clomipramine, dothiepin, doxepin, imipramine, nortriptyline, trimipramine, fentanyl, glycylxylidide, lidocaine, monoethylglycylxylidide, N-acetylprocainamide, procainamide, pregabalin, 2-Methylamino-1-(3,4-methylendioxyphenyl)butan. N-methyl-1,3-benzodioxolylbutanamine, 2-Amino-1-(3,4-methylendioxyphenyl)butan, 1,3-benzodioxolylbutanamine, normeperidine, O-Destramadol, desmetramadol, tramadol, lamotrigine, Theophylline, amikacin, gentamicin, tobramycin, vancomycin, Methotrexate. Gabapentin sisomicin and 5-methylcytosine.

In embodiments of the first aspect of the present invention, the analyte molecule is a carbohydrate or substance having a carbohydrate moiety, e.g. a glycoprotein or a nucleoside. In embodiments of the first aspect of the present invention, the analyte molecule is a monosaccharide, in particular selected from the group consisting of ribose, desoxyribose, arabinose, ribulose, glucose, mannose, galactose, fucose, fructose. N-acetylglucosamine, N-acetylgalactosamine, neuraminic acid, N-acetylneurominic acid, etc. In embodiments, the analyte molecule is an oligosaccharide, in particular selected from the group consisting of a disaccharide, trisaccharid, tetrasaccharide, polysaccharide. In embodiments of the first aspect of the present invention, the disaccharide is selected from the group consisting of sucrose, maltose and lactose. In embodiments of the first aspect of the present invention, the analyte molecule is a substance comprising above described mono-, di-, tri-, tetra-, oligo- or polysaccharide moiety.

In embodiments of the first aspect of the present invention, the analyte molecule comprises an azide group as functional group which is selected from the group consisting of alkyl or aryl azide. In embodiments of the first aspect of the present invention, the analyte molecule comprising one or more azide groups is selected from the group consisting of zidovudine and azidocillin.

Such analyte molecules may be present in biological or clinical samples such as body liquids, e.g. blood, serum, plasma, urine, saliva, spinal fluid, etc., tissue or cell extracts, etc. In embodiments of the first aspect of the present invention, the analyte molecule(s) are present in a biological or clinical sample selected from the group consisting of blood, serum, plasma, urine, saliva, spinal fluid, and a dried blood spot.

In some embodiments of the first aspect of the present invention, the analyte molecules may be present in a sample which is a purified or partially purified sample, e.g. a purified or partially purified protein mixture or extract.

In embodiments of the first aspect of the present invention, the reactive unit K is selected from the group consisting of a carbonyl reactive unit, a diene reactive unit, a hydroxyl reactive unit, an amino reactive unit, an imine reactive unit, a thiol reactive unit, a diol reactive unit, a phenol reactive unit, an epoxide reactive unit, a disulfide reactive unit, and an azido reactive unit.

In embodiments of the first aspect of the present invention, the reactive unit K is a carbonyl reactive unit, which is capable of reacting with any type of molecule having a carbonyl group. In embodiments of the first aspect of the present invention, the carbonyl reactive unit is selected from the group consisting of carboxyl reactive unit keto reactive unit, aldehyde reactive unit, anhydride reactive unit, carbonyl ester reactive unit, and imide reactive unit. In embodiments of the first aspect of the present invention, the carbonyl-reactive unit may have either a super-nucleophilic N atom strengthened by the α-effect through an adjacent O or N atom NH₂-N/O or a dithiol molecule.

In embodiments of the first aspect of the present invention, the carbonyl-reactive unit is selected from the group consisting of
(i) a hydrazine unit, e.g. a H₂N—NH—, or H₂N—NR1- unit, wherein R1 is aryl or C1-4 alkyl, particularly C1 or C2 alkyl, optionally substituted,
(ii) a hydrazide unit, in particular a carbo-hydrazide or a sulfohydrazide, in particular a H₂N—NH—C(O)—, or H₂N—NR2-C(O)— unit, wherein R2 is aryl or C1-4 alkyl, particularly C1 or C2 alkyl, optionally substituted,
(iii) a hydroxylamino unit, e.g. a H₂N—O— unit, and
(iv) a dithiol unit, particularly a 1,2-dithiol or 1,3-dithiol unit.

In embodiments of the first aspect of the present invention, wherein the carbonyl reactive unit is a carboxyl reactive unit, the carboxyl reactive units reacts with carboxyl groups on an analyte molecule. In embodiment of the first aspect of the present invention, the carboxyl reactive unit is selected from the group consisting of a diazo unit, an alkylhalide, amine, and hydrazine unit.

In embodiments of the first aspect of the present invention, analyte molecule comprises an ketone or aldehyde group and Q is a carbonyl reactive unit, which is selected from the group:
(i) a hydrazine unit.
(ii) a hydrazide unit,
(iii) a hydroxylamino unit, and
(iv) a dithiol unit.

In embodiments of the first aspect of the present invention, the reactive unit K is a diene reactive unit, which is capable of reacting with an analyte comprising a diene group. In embodiments of the first aspect of the present invention, the diene reactive unit is selected from the group consisting of Cookson-type reagents, e.g. 1,2,4-triazoline-3,5-diones, which are capable to act as a dienophile.

In embodiments of the first aspect of the present invention, the reactive unit K is a hydroxyl reactive unit, which is capable of reacting with an analyte comprising a hydroxyl group. In embodiments of the first aspect of the present invention, the hydroxyl reactive units is selected from the group consisting of sulfonylchlorides, activated carboxylic esters (NHS, or imidazolide), and fluoro aromates/heteroaromates capable for nucleophilic substitution of the fluorine (T. Higashi J Steroid Biochem Mol Biol. 2016 September; 162:57-69). In embodiments of the first aspect of the present invention, the reactive unit K is a diol reactive unit which reacts with an diol group on an analyte molecule. In embodiments of the first aspect of the present invention, wherein the reactive unit is a 1,2 diol reactive unit, the 1.2 diol reactive unit comprises boronic acid. In further embodiments, diols can be oxidised to the respective ketones or aldehydes and then reacted with ketone/aldehyde-reactive unit(s) K.

In embodiments of the first aspect of the present invention, the amino reactive unit reacts with amino groups on an analyte molecule. In embodiments of the first aspect of the present invention, the amino-reactive unit is selected from the group consisting of active ester group such as N-hydroxy succinimide (NHS) ester or sulfo-NHS ester, pentafluoro phenyl ester, cabonylimidazole ester, quadratic acid esters, a hydroxybenzotriazole (HOBt) ester, 1-hydroxy-7-azabenzotriazole (HOAt) ester, and a sulfonylchloride unit.

In embodiments of the first aspect of the present invention, the thiol reactive unit reacts with an thiol group on an analyte molecule. In embodiments of the first aspect of the present invention, the thiole reactive unit is selected from the group consisting of haloacetyl group, in particular selected from the group consisting of Br/I—CH₂—C(═O)— unit, acrylamide/ester unit, unsaturated imide unit such as maleimide, methylsulfonyl phenyloxadiazole and sulfonyichloride unit.

In embodiments of the first aspect of the present invention, the phenol reactive unit reacts with phenol groups on an analyte molecule. In embodiments of the first aspect of the present invention, the phenol-reactive unit is selected from the group consisting of active ester unit such as N-hydroxy succinimide (NHS) ester or sulfo-NHS ester, pentafluoro phenyl ester, carbonylimidazole ester, quadratic acid esters, a hydroxybenzotriazole (HOBt) ester, 1-hydroxy-7-azabenzotriazole (HOAt) ester, and a sulfonylchloride unit. Phenol groups present on an analyte molecule can be reacted with highly reactive electrophiles like triazolinedione (like TAD) via a reaction (H. Ban et al J. Am. Chem. Soc., 2010, 132 (5), pp 1523-1525) or by diazotization or alternatively by ortho nitration followed by reduction to an amine which could then be reacted with an amine reactive reagent. In embodiments of the first aspect of the present invention, the phenol-reactive unit is fluoro-1-pyridinium.

In embodiments of the first aspect of the present invention, the reactive unit K is a epoxide reactive unit, which is capable of reacting with an analyte comprising a epoxide group. In embodiments of the first aspect of the present invention, the epoxide reactive unit is selected from the group consisting of amino, thiol, super-nucleophilic N atom strengthened by the α-effect through an adjacent O or N atom NH₂—N/O molecule. In embodiments of the first aspect of the present invention, the epoxide reactive unit is selected from the group:
(i) a hydrazine unit, e.g. a H₂N—NH—, or H₂N—NR¹— unit, wherein R¹ is aryl, aryl containing one or more heteroatoms or C₁₋₄ alkyl, particularly C₁ or C₂ alkyl, optionally substituted e.g. with halo, hydroxyl, and/or C₁₋₃ alkoxy,
(ii) a hydrazide unit, in particular a carbo-hydrazide or sulfo-hydrazide unit, in particular a H₂N—NH—C(O)—, or H₂N—NR²—C(O)— unit,
   wherein R² is aryl, aryl containing one or more heteroatoms or C₁₋₄ alkyl, particularly C₁ or C₂ alkyl, optionally substituted e.g. with halo, hydroxyl, and/or C₁₋₃ alkoxy, and
(iii) a hydroxylamino unit, e.g. a H₂N—O— unit.

In embodiments of the first aspect of the present invention, the reactive unit K is a disulfide reactive unit, which is capable of reacting with an analyte comprising a disulfide group. In embodiments of the first aspect of the present invention, the disulfide reactive unit is selected from the group consisting of thiol. In further embodiments, disulfide group can be reduced to the respective thiol group and then reacted with thiol reactive units Q.

In embodiments of the first aspect of the present invention, the reactive unit K is a thiol-reactive group or is an amino-reactive group such as an active ester group, e.g. N-hydroxysuccinimide (NHS) ester or sulpho-NHS ester, a hydroxybenzotrialzole (HOBt) ester or 1-hydroxy-7-acabenzotriazole (OAt) ester group.

In embodiments of the first aspect of the present invention, the reactive unit K is selected from 4-substituted 1,2,4-triazolin-3,5-dione (TAD), 4-Phenyl-1,2,4-triazolin-3,5-dion (PTAD) or fluoro-substituted pyridinium.

In embodiments of the first aspect of the present invention, the reactive unit K is a azido reactive unit which reacts with azido groups on an analyte molecule. In embodiments of the first aspect of the present invention, the azido-reactive unit reacts with azido groups through azide-alkyne cycloaddition. In embodiments of the first aspect of the present invention, the azido-reactive unit is selected from the group consisting of alkyne (alkyl or aryl), linear alkyne or cyclic alkyne. The reaction between the azido and the alkyne can proceed with or without the use of a catalyst. In further embodiments of the first aspect of the present invention the azido group can be reduced to the respective amino group and then reacted with amino reactive units K.

In embodiments of the first aspect of the present invention, the functional group of the analyte is selected from the options mentioned in the left coloumn of the table 1. The reactive group of Q of the corresponding functional group of the analyte is selected from the group mentioned in the right coloumn of table 1.

TABLE 1
Functional group of the analyte and reactive
groups for the specific labels

| Functional group of | |
|---|---|
| the analyte | Reactive group |
| Amine | Active ester with NHS leaving group, |
| | pentafluorophenyl ester, squaric acid esters, |
| | sulfonyl chloride, |
| | ketone or aldehyde (reductive amination) |
| Thiol | Maleimide, iodoacetyl, methylsulfonyl |
| | phenyloxadiazole |
| Diol | Boronic acid (or oxidation to ketone or |
| | aldehyde) |
| Ketone, aldehyde | O-substituted hydroxylamine, hydrazines, |
| | hydrazides. |
| Diene | Dienophiles, triazolinedione (TAD) |
| Phenoles | Ene reaction triazolinedione (TAD), ortho |
| | nitration/reduction, diazo |
| | formation/nucleophilic substitution. |
| | Active ester with NHS leaving group, |
| | pentafluorophenyl ester, squaric acid esters, |
| | sulfonyl chloride, fluoro-1-pyridinium. |
| Nucleobase | Chloro acetyl/Pt complexes |
| Unspecific | Azide (Nitrene) |
| Carboxylic acids | EDAC activation => amine |
| | Base/alkyl halide |
| | Chloroformate/alcohol |
| | Diazoalkane |
| Terminal cysteine | Hetero aryl/Aryl cyanides |
| Terminal serine | Oxidation (followed by aldehyde reactive |
| | reagents) |

In embodiments of the first aspect of the invention, the analyte of interest is free of a carbonyl group. The analyte of interest does not comprise a carbonyl group.

According to step (c), the pretreated sample is diluted. Step (c) can be performed after step (a) and/or step (b). Alternatively, at least steps (b) and (c) are performed simultaneously. Preferably, step (c) can not be performed before step (b). More preferably, step (c) of the method of determining the level of Testosterone can not be performed before step (b) by said method. The term “simultaneously” can mean in this context that steps (b) and (c) are performed or are done at the same time or time period, in particular exactly at the same time or time period. This can mean that steps (b) and (c) have the same starting point and/or ending point. Alternatively, the starting point and/or ending point of the two steps can differ. e.g. with a tolerance of 40% or 30% or 20% or 10% or 5% or 3% or 2% or 1% or 0.5%.

In embodiments of the first aspect of the invention, step c) is performed after step b).

In embodiments of the first aspect of the invention, the sample in step c) is diluted by a solvent or a mixture of solvents.

In embodiments of the first aspect of the invention, the solvent is an electron spray suitable solvent.

In embodiments of the first aspect of the invention, the solvent is selected from the group consisting of water, methanol, acetonitrile or mixtures thereof. The solvent or mixtures of solvents can comprise additional additives for improving the nanoESI process. e.g. formic acid, e.g. 0.1% formic acid.

In embodiments of the first aspect of the invention, the pretreated sample is diluted in step c) in such a way that the dilution factor of the analyte of interest to the compound is in the range from 1:0.001 to 1:1000. Preferably, the dilution factor of the analyte of interest to the compound is in the range from 1:0.1 to 1:1 or 1:0.1 to 1:10 or 1:10 to 1:20 or 1:10 to 1:50 or 1:30 to 1:70.

In embodiments of the first aspect of the invention, the pretreated sample is diluted in step c) in such a way that the dilution factor of the analyte of interest to the compound is in the range from 1:1 to 1:100.

In embodiments of the first aspect of the invention, the pretreated sample is diluted in step c) in such a way that the level of the analyte is by factor 1:1000, preferably 1:100 or 1:10 higher than the level of the analyte in step (b).

According to step (d), the level of the analyte of interest in the pretreated sample is determined by using nanoESI mass spectrometry.

The quantitative analysis according to step (d) of is carried out by mass spectrometry (MS). Preferably, the MS analysis procedure comprises a tandem MS (MS/MS) analysis, particularly a triple quadrupole (Q) MS/MS analysis. Additionally, the MS comproses a nanoESI as an ionization source. A skilled person knows nanoESI as an ionization source. Therefore, it is not further explained at this point.

In embodiments of the first aspect of the invention, the nanoESI mass spectrometry is static.

Surprisingly, it was found that a combination of a derivatizing step and diluting step in a method, the level of the analyte of interest can be determined by using nanoESI MS in a sensitive manner. In the described invention solution, the advantages of nanoESI regarding better ion yields are combined with the possibility to derivatized the target analyte with specific reagents which additionally increase the ion yields. Due to a reduction of ionization competition and the low material input into the ion source, a lower contamination of the whole system can be assumed.

Alternative substances can be added to the solvent of the pretreated sample to improve the signal, e.g. a dopand spray like an acid, a base. DMSO or toluene. As an acid an organic acid. e.g. formic acid, can be used. Ammoniumacetate or NH₄OH can be used as a base.

This method can be used to increase the sensitivity of the entire system so that the patient sample together with the analyte can be diluted in a suitable solvent. This is contrary to the state of the art where the analyte must be further concentrated in the process to enable mass spectrometric detection.

By combining derivatisation, dilution and nanoESI it is possible to perform quantitative MS determinations of even very low concentrated analytes such as steroids in serum without the use of HPLC separation columns.

Very low concentrated analytes can mean in this context, concentrations in the μg/mL range, i.e. in the range from 1 μg/ml to 999 μg/ml.

Surprisingly, the combination of derivatization and static nanoESI leads to signal amplification that is significantly higher than the expected combination of the individual components.

Advantages of the solution according to the first aspect of the invention compared to HPLC-MS are:
1. reduced complexity and robustness
   Extremely reduced solvent consumption (e.g. factor 3500 in comparison to 700 μl/min flow rate)
   Significantly less substance entry into the mass spectrometer (e.g. factor>1000; nl, instead of μL sample volume)
   Maintenance effort MS reduced due to less contamination
   No carryover when using “single use spray nozzles”
   For analytes in the higher concentration range (e.g. TDMs) a low end MS can be used and thus the hardware costs can be reduced
   No need for fast scanning MS hardware
2. simplified workflow
   Simple sample preparation (bead separation or protein precipitation)
   Derivatizing instead of concentrating
   Dilution instead of concentration/depletion
   No gradient HPLC necessary
   No HPLC separation column necessary
   Separation of isobars by ion mobility or immunosorption on beads or similar active surfaces e.g. C18 material capture zone etc.
3. improved performance
   Synergistic effects of nano-ESI and derivatization
   Specific for functional groups by derivatisation
   Variable residence time of the analyte in the ion source
   Increase of the available measuring time in the MS
   Possibility of multiple MS experiments
   Improvement of the S/N ratio (signale to noise ratio)
   Improvement of detection limits

In a second aspect, the present invention relates to the use of the method of the first aspect of the present invention for determining the level of an analyte of interest in a pretreated sample. All embodiments mentioned for the first aspect of the invention apply for the second aspect of the invention and vice versa.

In a third aspect, the present invention relates to a diagnostic system for determining a level of an analyte of interest in a pretreated sample, comprising a nanoESI source and a mass spectrometer to carry out the method according to the first aspect of the invention. All embodiments mentioned for the first aspect of the invention and/or second aspect of the invention apply for the third aspect of the invention and vice versa.

In embodiments of the third aspect of the present invention, diagnostic system is a clinical diagnostic system.

In embodiments of the third aspect of the present invention, the nanoESI source can be e.g. a chip-based electrospray ionization technology from company Advion. It combines the benefits of liquid chromatography, mass spectrometry, chip-based infusion, fraction collection, and direct surface analysis into one integrated ion source platform. Other known nanoESI sources are also possible. The nanoESI source is known for a skilled person and therefore not explained in detail.

In embodiments of the third aspect of the present invention, the mass spectrometer can be e.g. a triple quadrupole mass spectrometer or a linear ion trap mass spectrometer. A mass spectrometer is known for a skilled person and thus not explained in detail.

A “clinical diagnostics system” is a laboratory automated apparatus dedicated to the analysis of samples for in vitro diagnostics. The clinical diagnostics system may have different configurations according to the need and/or according to the desired laboratory workflow. Additional configurations may be obtained by coupling a plurality of apparatuses and/or modules together. A “module” is a work cell, typically smaller in size than the entire clinical diagnostics system, which has a dedicated function. This function can be analytical but can be also pre-analytical or post analytical or it can be an auxiliary function to any of the pre-analytical function, analytical function or post-analytical function. In particular, a module can be configured to cooperate with one or more other modules for carrying out dedicated tasks of a sample processing workflow, e.g. by performing one or more pre-analytical and/or analytical and/or post-analytical steps. In particular, the clinical diagnostics system can comprise one or more analytical apparatuses, designed to execute respective workflows that are optimized for certain types of analysis, e.g. clinical chemistry, immunochemistry, coagulation, hematology, liquid chromatography separation, mass spectrometry, etc. Thus the clinical diagnostic system may comprise one analytical apparatus or a combination of any of such analytical apparatuses with respective workflows, where pre-analytical and/or post analytical modules may be coupled to individual analytical apparatuses or be shared by a plurality of analytical apparatuses. In alternative pre-analytical and/or post-analytical functions may be performed by units integrated in an analytical apparatus. The clinical diagnostics system can comprise functional units such as liquid handling units for pipetting and/or pumping and/or mixing of samples and/or reagents and/or system fluids, and also functional units for sorting, storing, transporting, identifying, separating, detecting.

The clinical diagnostic system can comprise a sample preparation station for the automated preparation of samples comprising analytes of interest, optionally a liquid chromatography (LC) separation station comprising a plurality of LC channels and/or a sample preparation/LC interface for inputting prepared samples into any one of the LC channels. In particular, the clinical diagnostic system is free of a separation station, e.g. a LC-HPLC unit or HPLC unit.

The clinical diagnostic system can further comprise a controller programmed to assign samples to pre-defined sample preparation workflows each comprising a pre-defined sequence of sample preparation steps and requiring a pre-defined time for completion depending on the analytes of interest. The clinical diagnostic system can further comprise a mass spectrometer (MS) and an LC/MS interface for connecting the LC separation station to the mass spectrometer.

A “sample preparation station” can be a pre-analytical module coupled to one or more analytical apparatuses or a unit in an analytical apparatus designed to execute a series of sample processing steps aimed at removing or at least reducing interfering matrix components in a sample and/or enriching analytes of interest in a sample. Such processing steps may include any one or more of the following processing operations carried out on a sample or a plurality of samples, sequentially, in parallel or in a staggered manner: pipetting (aspirating and/or dispensing) fluids, pumping fluids, mixing with reagents, incubating at a certain temperature, heating or cooling, centrifuging, separating, filtering, sieving, drying, washing, resuspending, aliquoting, transferring, storing, etc.).

A “liquid chromatography (LC) separation station” is an analytical apparatus or module or a unit in an analytical apparatus designed to subject the prepared samples to chromatographic separation in order for example to separate analytes of interest from matrix components, e.g. remaining matrix components after sample preparation that may still interfere with a subsequent detection, e.g. a mass spectrometry detection, and/or in order to separate analytes of interest from each other in order to enable their individual detection. According to an embodiment, the LC separation station is an intermediate analytical apparatus or module or a unit in an analytical apparatus designed to prepare a sample for mass spectrometry and/or to transfer the prepared sample to a mass spectrometer. In particular, the LC separation station is a multi-channel LC station comprising a plurality of LC channels. Preferably, the clinical diagnostic system is free of the liquid chromatography (LC) separation station.

The clinical diagnostic system, e.g. the sample preparation station, may also comprise a buffer unit for receiving a plurality of samples before a new sample preparation start sequence is initiated, where the samples may be individually randomly accessible and the individual preparation of which may be initiated according to the sample preparation start sequence.

The clinical diagnostic system makes use of LC coupled to mass spectrometry more convenient and more reliable and therefore suitable for clinical diagnostics. In particular, high-throughput, e.g. up to 100 samples/hour or more with random access sample preparation and LC separation can be obtained while enabling online coupling to mass spectrometry. Moreover the process can be fully automated increasing the walk-away time and decreasing the level of skills required.

In a fourth aspect, the present invention relates to the use of the diagnostic system of the third aspect of the invention in the method of the first aspect of the invention. All embodiments mentioned for the first aspect of the invention and/or second aspect of the invention and/or third aspect of the invention apply for the fourth aspect of the invention and vice versa.

In a fifth aspect, the present invention relates to a kit suitable to perform a method of the first aspect of the invention comprising
(i) a compound for derivatizing the analyte of interest in a pretreated sample, wherein the compound is capable of forming a covalent bond to the analyte of interest,
(ii) a solvent or mixtures of solvents for diluting the pretreated sample comprising the derivatized analyte of interest, and
(iii) optionally a catalyst. All embodiments mentioned for the first aspect of the invention and/or second aspect of the invention and/or third aspect of the invention and/or fourth aspect of the invention apply for the fifth aspect of the invention and vice versa.

In embodiments of the fifth aspect of the present invention, the solvent or mixtures of solvents for diluting the pretreated sample are selected from the group consisting of water, organic solvents e.g methanol, acetonitrile, and mixtures of water and at least one organic solvent.

In embodiments of the fifth aspect of the present invention, the kit comprises a catalyst. The catalyst makes a chemical reaction happen more quickly without itself being changed. In particular, the catalyst is a chemical substance. The catalyst is, for example, a lewis base.

In a sixth aspect, the present invention relates to a the use of a kit of the fifth aspect of the invention in a method of the first aspect of the invention.

In further embodiments, the present invention relates to the following aspects:

1. A method of determining the level of an analyte of interest in a pretreated sample comprising the following steps:
a) Providing the pretreated sample, in particular the pretreated sample of bodily fluid including the analyte of interest,
b) Derivatizing the analyte of interest, preferably in the pretreated sample, c) Diluting the pretreated sample, and
d) Determining the level of the analyte of interest in the pretreated sample using nanoESI mass spectrometry.

2. The method of aspect 1, wherein the method is free of a further step after performing step a) or step b), wherein the further step is selected from the group consisting of extraction step, chromatographic step, lyophilization, centrifuge or combinations thereof.

3. The method of aspect 2, wherein the chromatographic step comprises at least one or more methods selected from the following group: chromatography, high performance liquid chromatography (HPLC), liquid chromatography high performance liquid chromatography (LC-HPLC), gel permeation chromatography (GPC), flash chromatography, wherein chromatography is, for example, size exclusion chromatography.

4. The method of aspect 2, wherein the extraction step comprises at least one or more methods selected from the following group: liquid-liquid extraction, liquid-solid extraction, liquid-gas extraction, gas-liquid extraction, solid-liquid extraction, solid phase extraction (SPE).

5. The method of any of the proceeding aspects, wherein the method is automated.

6. The method of any of the proceeding aspects, wherein the pretreated sample is obtained from a patient sample, which is selected from a group consisting of serum, plasma and whole blood sample from an individual.

7. The method of any of the proceeding aspects, wherein the pretreated sample is a hemolysed whole-blood sample, particularly a hemolysed human whole-blood sample.

8. The method of any of the proceeding aspects, wherein the pretreated sample is free of a tissue sample or wherein the pretreated sample is not a tissue sample.

9. The method of any of the proceeding aspects, wherein the pretreated sample is obtained by at least one or more pre-treatment steps and/or by at least one or more enrichment steps.

10. The method of any of the proceeding aspects, wherein at least one enrichment step comprises a chemical precipitation or a solid phase, wherein in particular the solid phase is a bead, wherein the bead is magnetic or paramagnetic.

11. The method of any of the proceeding aspects, wherein the method is an in vitro method.

12. The method of any of the proceeding aspects, wherein step b) is performed at a temperature, which is at least 20° C. or more.

13. The method of any of the proceeding aspects, wherein step b) is performed at least at 30° C., for example 35° C.

14. The method of any of the proceeding aspects, wherein step b) is performed at least at 40° C., for example 45° C.

15. The method of any of the proceeding aspects, wherein step b) is performed at least at 50° C., for example 55° C.

16. The method of any of the proceeding aspects, wherein step b) is performed at least at 60° C., for example 65° C.

17. The method of any of the proceeding aspects, wherein step b) is performed at least at 70° C., for example 75° C.

18. The method of any of the proceeding aspects, wherein step b) is performed at least at 80° C., for example 85° C.

19. The method of any of the proceeding aspects, wherein step b) comprises the addition of a further substance or further substances, e.g. additives, wherein the further substance or the further substances are e.g. for protonation and/or for catalysis, in particular wherein the further substance for catalysis is a lewis base.

20. The method of any of the proceeding aspects, wherein the analyte of interest is derivatized in step b) by a compound, which is capable of forming a covalent binding to the analyte of interest, in particular wherein after step b) the compound is covalently bounded to the analyte of interest for forming a complex with the analyte of interest.

21. The method of any of the proceeding aspects 20, wherein the compound is simple permanent positively charged or simple permanent negatively charged.

22. The method of any of the proceeding aspects 20, wherein the compound is double permanent positively charged or double permanent negatively charged.

23. The method of any of the proceeding aspects 20, wherein the compound is free of a permanent charge.

24. The method of any of the proceeding aspects 20 to 23, wherein the ratio of the analyte of interest to the compound is in the range of 1:1 to 1:6.000.000 in step b).

25. The method of any of the proceeding aspects 20 to 24, wherein the compound comprises a reactive unit K, which is able of reacting with a carbonyl group, phenol group, amine, hydroxyl group or diene group of the analyte of interest.

26. The method of any of the proceeding aspects 20 to 25, wherein K is selected from the group consisting of hydrazide, hydrazine, hydroxylamine. Br, F-aromatic, 4-substituted 1,2,4-triazolin-3,5-dione (TAD), active ester, sulfonylchloride and reactive carbonyl.

27. The method of any of the proceeding aspects 20 to 26, wherein the compound comprises a counter ion for forming a salt, wherein the counter ion is preferably selected from the following group: Cl⁻, Br⁻, F⁻, formiate, trifluoroacetate, PF₆⁻, sulfonate, phosphate, acetate.

28. The method of any of the proceeding aspects 20 to 27, wherein the compound comprises a permanent charge, in particular a permanent net charge, wherein said compound is capable of covalently binding to the analyte of interest,
wherein said compound has a mass m1 and a net charge z1.
wherein the compound is capable of forming at least one daughter ion having a mass m2<m1 and a net charge z2<z1 after fragmentation by mass spectrometric determination,
wherein m1/z1<m2/z2.

29. The method of any of the proceeding aspects 20 to 28, wherein the compound comprises formula A or B:

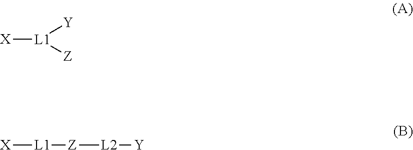

wherein
X is a reactive unit, which is in particular capable of forming a covalent bond with an analyte of interest,
L1 and L2 are independently of each other substituted or unsubstituted
linker, in particular branched or linear linker,
Y is a neutral loss unit, and
Z is a charged unit comprising at least one permanently charged moiety, in particular comprising one permanently charged moiety, including any salt thereof.

30. The method of any of the proceeding aspects 20 to 29, wherein the compound is selected from the group consisting of: dansylchloride, carbamic acid, N-[2-[[[2-(diethylamino)ethyl]amino]carbonyl]-6-quinolinyl]-, 2.5-dioxo-1-pyrrolidinyl ester (RapiFluor-MS), 4-substituted 1,2,4-triazoline-3,5-diones (Cookson-type reagents), 4-Phenyl-1,2,4-triazolin-3,5-dion-derivative (Amplifex Diene), 1-propanaminium, 3-(aminooxy)-N,N,N-trimethyl-compound comprising an appropriate counter ion, e.g. bromide, chloride, iodine, etc. (Amplifex Keto), acethydrazide trimethylammonium chloride (Girard T), 1-(carboxymethyl)pyridiniun chloride hydrazide (Girard P) and pyridiyl amine.

31. The method of any of the proceeding aspects 20 to 30, comprising the compound of formula P1:

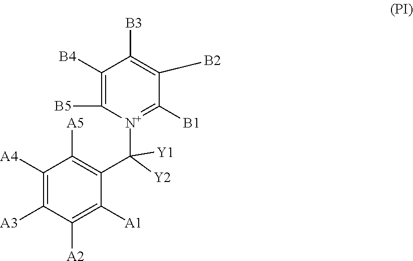

wherein one of the substituents B1, B2, B3, B4. B5 is a coupling group Q, which is capable of forming a covalent bond with the analyte,
   wherein the other substituents A1, A2, A3. A4. A5, B1, B2, B3, B4, B5 are each independently selected from hydrogen, halogen, alkyl, N-acylamino, N,N-dialkylamino, alkoxy, thioalkoxy, hydroxy, cyano, alkoxycarbonyl, alkoxythiocarbonyl, acyl, nitro, thioacyl, aryloyl, fluoromethyl, difluoromethyl, trifluoromethyl, trifluoroethyl, cyanomethyl, cyanoethyl, hydroxyethyl, methoxyethyl, nitroethyl, acyloxy, aryloyloxy, cycloalkyl, aryl, heteroaryl, heterocycloalkyl, amino, isotope or derivative thereof,
wherein Y1 and Y2 are each independently selected from hydrogen, methyl, ethyl, methoxy, substituted aromatic, unsubstituted aromatic, substituted cycloalkyl, unsubstituted cycloalkyl, substituted heteroaromatic, unsubstituted heteroaromatic, amine or wherein Y1 and Y2 form a ring structure, which is selected from substituted cycloalkyl, unsubstituted cycloalkyl, substituted aromatic, unsubstituted aromatic, substituted heteroaromatic, unsubstituted heteroaromatic.

32. The method of any of the proceeding aspects 20 to 31, comprising the compound of formula D1:

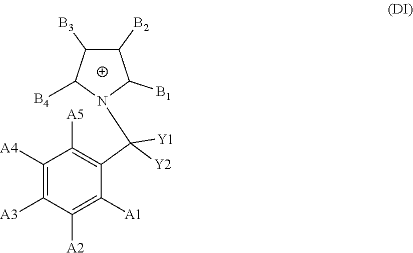

wherein one of the substituents B1. B2, B4 is a coupling group Q, which is capable of forming a covalent bond with the analyte,
wherein the other substituents A1, A2, A3, A4, A5, B1, B2, B4 are each independently selected from hydrogen, halogen, alkyl, N-acylamino, N,N-dialkylamino, alkoxy, thioalkoxy, hydroxy, cyano, alkoxycarbonyl, alkoxythiocarbonyl, acyl, nitro, thioacyl, aryloyl, fluoromethyl, difluoromethyl, trifluoromethyl, trifluoroethyl, cyanomethyl, cyanoethyl, hydroxyethyl, methoxyethyl, nitroethyl, acyloxy, aryloyloxy, cycloalkyl, aryl, heteroaryl, heterocycloalkyl, amino, isotope or derivative thereof,
wherein B3 is selected from alkyl, acetyl, vinyl, substituted aromatic, unsubstituted aromatic, substituted benzyl, unsubstituted benzyl, substituted cycloalkyl, unsubstituted cycloalkyl, isotope and derivative thereof,
wherein Y1 and Y2 are each independently selected from hydrogen, methyl, ethyl, methoxy, substituted aromatic, unsubstituted aromatic, substituted cycloalkyl, unsubstituted cycloalkyl, substituted heteroaromatic, unsubstituted heteroaromatic, amine or wherein Y1 and Y2 form a ring structure, which is selected from substituted cycloalkyl, unsubstituted cycloalkyl, substituted aromatic, unsubstituted aromatic, substituted heteroaromatic, unsubstituted heteroaromatic.

33. The method of any of the proceeding aspects 20 to 32, comprising the compound of formula C1:

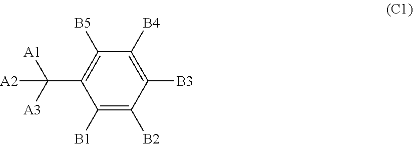

wherein one of the substituents B1, B2, B3, B4, B5 is a coupling group Q, which is capable of forming a covalent bond with the analyte,
wherein the other substituents A1, A2, B1, B2, B3, B4, B5 are each independently selected from hydrogen, halogen, alkyl, modified alkyl, N-acylamino, N,N-dialkylamino, alkoxy, thioalkoxy, hydroxy, cyano, alkoxycarbonyl, alkoxythiocarbonyl, acyl, nitro, thioacyl, aryloyl, fluoromethyl, difluoromethyl, trifluoromethyl, trifluoroethyl, cyanomethyl, cyanoethyl, hydroxyethyl, methoxyethyl, nitroethyl, acyloxy, aryloyloxy, cycloalkyl, aryl, heteroaryl, heterocycloalkyl, amino, sulfur, isotope or derivative thereof,
wherein A3 comprises ammonium, pyridinium, phosphonium or derivatives thereof, wherein in case of A3 is ammonium and B or B5 is the coupling group Q, the coupling group Q comprises a C atom, which is separated by four single or double bonds from the C atom of the CA1A2A3 substituent and the coupling group Q comprises a C-atom, which is separated by five single or double bonds from the C atom of the CA1A2A3 substituent.

34. The method of any of the proceeding aspects, wherein the analyte of interest is selected from the group consisting of nucleic acid, amino acid, peptide, protein, metabolite, hormones, fatty acid, lipid, carbohydrate, steroid, ketosteroid, secosteroid, a molecule characteristic of a certain modification of another molecule, a substance that has been internalized by the organism, a metabolite of such a substance and combination thereof.

35. The method of any of the proceeding aspects, wherein the analyte of interest is free of a carbonyl group.

36. The method of any of the proceeding aspects, wherein step c) is performed after step b).

37. The method of any of the proceeding aspects, wherein the sample in step c) is diluted by a solvent or a mixture of solvents.

38. The method of any of the proceeding aspects, wherein the solvent is an electron spray suitable solvent.

39. The method of any of the proceeding aspects, wherein the solvent is selected from the group consisting of water, methanol, acetonitrile or mixtures thereof.

40. The method of any of the proceeding aspects, wherein the pretreated sample is diluted in step c) in such a way that the dilution factor of the analyte of interest to the compound is in the range from 1:0.001 to 1:1000.

41. The method of any of the proceeding aspects, wherein the pretreated sample is diluted in step c) in such a way that the dilution factor of the analyte of interest to the compound is in the range from 1:1 to 1:10000, preferably 1:10 to 1:10000, more preferably 1:10 to 1:1000.

42. The method of any of the proceeding aspects, wherein the nanoESI mass spectrometry is static.

43. Use of the method of any one of aspects 1 to 42 for determining the level of an analyte of interest in a pretreated sample.

44. A diagnostic system for determining a level of an analyte of interest in a pretreated sample, comprising a nanoESI source and a mass spectrometer to carry out the method according to any one of aspects 1 to 42.

45. Use of the diagnostic system of aspect 44 in the method of any one of aspects 1 to 42.

46. A kit suitable to perform a method of any one of aspects 1 to 42 comprising
(i) a compound for derivatizing the analyte of interest in a pretreated sample, wherein the compound is capable of forming a covalent bond to the analyte of interest.
(ii) a solvent or mixtures of solvents for diluting the pretreated sample comprising the derivatized analyte of interest, and
(iii) optionally a catalyst.

47. Use of a kit of aspect 46 in a method of any one of aspects 1 to 42.

### EXAMPLES

The following examples are provided to illustrate, but not to limit the presently claimed invention.

### Example 1: Analytes in Neat Solution

¹³C3-Testosterone: mass concentration: 1 mg/mL in Methanol

Mz2974: mass concentration: 1 mg/mL in Methanol, considering the molar ratio of testosterone relatively to the testosterone derivate Mz2974 (molar mass of testosterone/molar mass of Mz2974=0.49) and the purity of Mz2974 of 0.90 detected by qNMR, the actual testosterone-mass concentration in Mz2974 stock solution is calculated 0.442 mg/mL (1 mg/mL*0.90*0.49=0.442 mg/mL testosterone ratio). All subsequent dilutions of Mz2974 are corrected accordingly by this factor.

The structures of ¹³C₃-Testosterone and Mz2974 are:

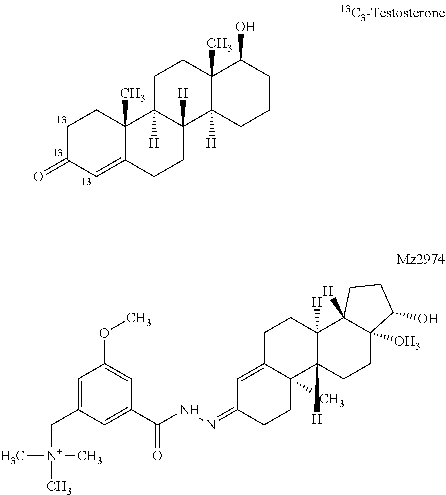

Subsequently, a 10 μg/mL stock solution #2 was prepared for following dilutions (solvent: H₂O/acetonitrile 70/30, +0.1% formic acid).

Testosterone-Girard T: mass concentration: 1 mg/mL in Methanol, considering the molar ratio of testosterone relatively to the testosterone derivate Girard T-testosterone (molar mass of testostcrone/molar mass of Testosterone-Girard T=˜ 0.65) and the purity of Testosterone-Girard T of 0.86 detected by qNMR, the actual testosterone-mass concentration in Testosterone-Glirard T stock solution is calculated 0.559 mg/mL (1 mg/mL 0.86*0.65=0.559 mg/mL testosterone ratio). All subsequent dilutions of Testosterone-Girard T are corrected accordingly by this factor.

Subsequently, a 10 μg/mL stock solution #t2 was prepared for following dilutions (solvent: H₂O/Acetonitrile 70/30, +0.1% formic acid).

n-Decylbenzamide: mass concentration: 1 mg/mL in Methanol

An analyte mixture with analyte concentrations of 1 μg/mL ¹³C₃-Testosteron, 1 μg/mL, Mz2974, 1 μg/mL, Testosteron-Girard T, and 100 ng/mL n-Decylbenzamide for internal standard use was prepared. The following calibrators were made by alternating dilution with 10% H2O, +0.1% formic acid, +100 ng/mL n-Decylbenzamide in acetonitrile:

| | |
|---|---|
| | ISTD |
| Analyte concentrations based on testosterone | (n-Decylbenzamide) |

| | | |
|---|---|---|
| 1000 | ng/mL | 100 ng/mL |
| 500 | ng/mL | 100 ng/mL |
| 100 | ng/mL | 100 ng/mL |
| 50 | ng/mL | 100 ng/mL |
| 10 | ng/mL | 100 ng/mL |
| 5 | ng/mL | 100 ng/mL |
| 1 | ng/mL | 100 ng/mL |
| 0.5 | ng/mL | 100 ng/mL |
| 0.1 | ng/mL | 100 ng/mL |
| 0.05 | ng/mL | 100 ng/mL |
| 0.01 | ng/mL | 100 ng/mL |

A Thermo LTQ mass spectrometer equipped with an Advion Triversa Nanomate ionization source was used for the measurements. The intensity of the signal of each analyte was summed up for the duration of 3 minutes. The relative intensity is defined as the ratio of the intensity of the analyte and the internal standard.

Advion Triversa Nanomate Ionization Source:

The parameters of the Advion Triversa Nanomate were optimized as follows:
Volume: 5 μL
Gas pressure: 0.6 psi
Voltage: 1.2 kV

Thermo LTQ Mass Spectrometer:

The Thermo LTQ mass spectrometer was operated in positive ionization mode. The acquisition time was set to 3 minutes. The parameters of the mass spectrometer were optimized as follows: capillary temperature, 250° C.; capillary voltage, 36 V: and tube lens, 70 V.

For all analytes and the internal standard, multiple reaction monitoring was performed. The collision energies for multiple reaction monitoring were optimized for highest signal intensities. The acquired mass transitions were as follows:
Testosterone-Girard T: nil 402.3→m/z 343.2 (Collision energy: 30)
Mz 2974: m1:508.3→m/z 449.3 (Collision energy: 28)
¹³C₃-Testosterone: m/z 292.2 m/z: 100.1 (Collision energy: 27)
n-Decylbenzamide: m/z 262.2 m/z: 105.0 (Collision energy: 35)

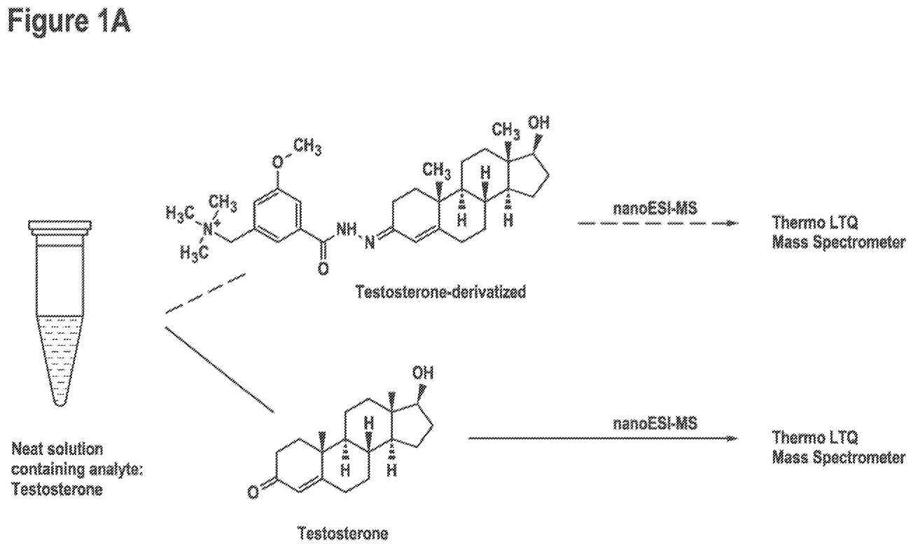

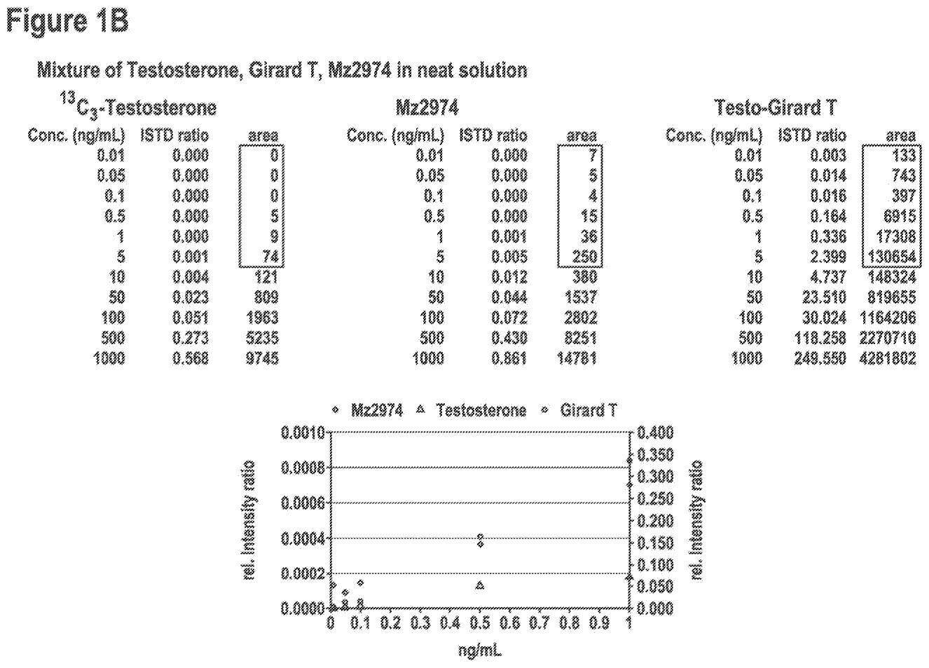

FIG. 1A shows two methods of determining the level of analyte of interest in a neat solution. The analyte of interest is in this case testosterone. In one method, the analyte is provided in a derivatised form by a compound Girard T or Mz2974 and then the level of the analyte of interest is determined in the pretreated sample using nanoESI mass spectrometry. In contrast to that, the other method shows the determining of the level of the analyte of interest (testosterone) sample using nanoESI mass spectrometry without a pre-derivatizing step.

Result in Neat Solution:

The defined mass transitions of ¹³C₃-Testosterone, Mz2974. Testosterone-GirardT, and n-Decylbenzamide, for internal standard use, were analyzed over a broad range of analyte concentrations ranging from 0.01 ng/mL to 1000 ng/mL in a neat solution matrix.

Especially, at low analyte concentrations from 0.01-1 ng/mL the summed signal area over a time period of 3 min for ¹³C₃-Testosterone was comparably very low. No ¹³C₃-Testosterone-signal was detected at concentrations from 0.01 ng/mL to 0.1 ng/mL. A constant signal of ¹³C₃-Testosterone was detected starting from 5 ng/mL to higher concentrations.

In contrast to these findings, a signal for Mz2974 and Girard T-derivatized Testosterone was detected over the full concentration range. Even at very low analyte concentrations where ¹³C₃-Testosterone was not detectable directly, the derivatized testosterone showed clearly a corresponding signal. Comparing the signal-intensity at the concentration of e.g. 1 ng/mL, Mz2974 shows a 4 fold, and Testosterone-Girard T a 1923 fold increase in the area of the signal.

FIG. 1B shows the results of these two methods. It is shown the relative intensity and areas, respectively, as a function of the concentration of underivatized Testosterone and derivatized Testosterone in neat solution. As a derivatizing reagent Girard T and Mz2974 were used. Underivatized Testosterone is not or marginal detectable, in particular at low concentrations of 5 ng/ml or lower. The derivatised analyte of interest in the pretreated sample leads to an increasing of the sensitivity. Comparing the intensity at the concentration of e.g. 1 ng/mL, Mz2974 shows a 4 fold, and Testosterone-Girard T a 1923 fold increase in the area of the signal. The structure of Mz2974 is:

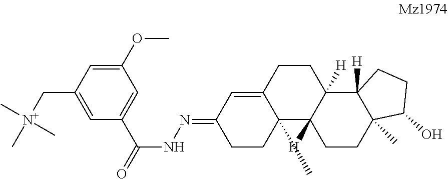 [SMILES: COc1cc(C[N+](C)(C)C)cc(C(=O)N/N=C2/C=C3CC[C@H]4[C@@H]5CC[C@H](O)[C@@]5(C)CC[C@@H]4[C@@]3(C)CC2)c1]

### Example 2: Analytes in Depleted Horse Serum Matrix

Protein Precipitation in Horse Serum:

The horse serum matrix (Sigma, H0146) was precipitated by addition of ice-cold methanol (−20° C.) in the ratio 1:5, mixed on a vortex mixer and subsequently centrifuged for 15 min at 5300 rpm (centrifuge Heraeus Megafuge 16R. Thermo Scientific). The supernatant was transferred and stored at −20° C. until usage.

For the matrix stock solution an analyte mixture with analyte concentrations of 1 μg/ml. ¹³C₃-Testosterone, 1 μg/mL Mz2974, 1 μg/mL Testosteron-Girard T, and 100 ng/mL n-Decylbenzamide was prepared in the MeOH-depleted horse serum matrix.

The following calibrators were made by alternating dilution with the MeOH-depleted horse serum matrix:

| | |
|---|---|
| | ISTD |
| Analyte concentrations based on testosterone | (n-Decylbenzamide) |

| | | |
|---|---|---|
| 1000 | ng/mL | 100 ng/mL |
| 500 | ng/mL | 100 ng/mL |
| 100 | ng/mL | 100 ng/mL |
| 50 | ng/mL | 100 ng/mL |
| 10 | ng/mL | 100 ng/mL |
| 5 | ng/mL | 100 ng/mL |
| 1 | ng/mL | 100 ng/mL |
| 0.5 | ng/mL | 100 ng/mL |
| 0.1 | ng/mL | 100 ng/mL |
| 0.05 | ng/mL | 100 ng/mL |
| 0.01 | ng/mL | 100 ng/mL |

A Thermo LTQ mass spectrometer equipped with an Advion Triversa Nanomate ionization source was used for the measurements of the calibrators. The intensity of the signal of each analyte was summed up for the duration of 3 minutes. The relative intensity is defined as the ratio of the intensity of the analyte and the internal standard.

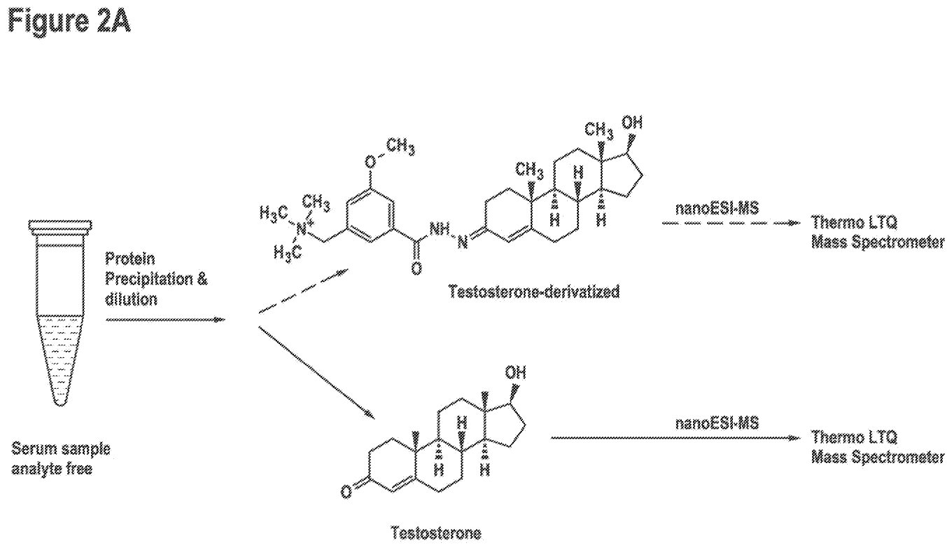

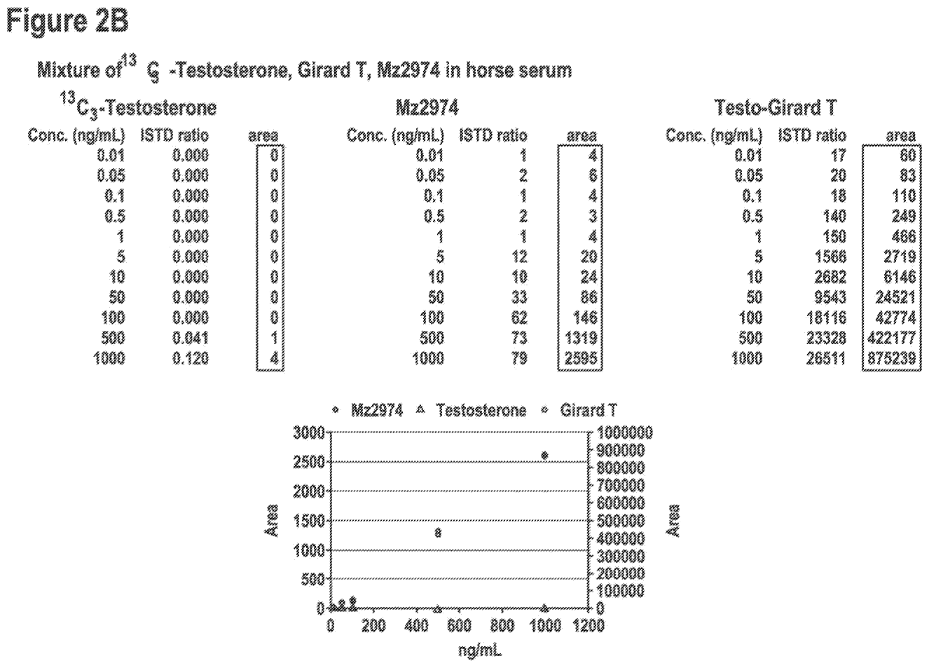

FIG. 2A shows two methods of determining the level of analyte of interest in a MeOH-depleted horse serum matrix solution. The analyte of interest is in this case testosterone. In one method, the analyte is provided in a derivatised form by a compound Girard T or Mz2974 and then the level of the analyte of interest is determined in the pretreated sample using nanoESI mass spectrometry. In contrast to that, the other method shows the determining of the level of the analyte of interest (testosterone) sample using nanoESI mass spectrometry without a pre-derivatizing step.

Result in MeOH-Depleted Horse Serum:

The defined mass transitions of ¹³C₃-Testosterone, Mz2974, Testosterone-GirardT, and n-Decylbenzamide, for internal standard use, were analyzed over a broad range of analyte concentrations ranging from 0.01 ng/mL to 1000 ng/mL in a MeOH-depleted horse serum matrix.

The summed signal area over a time period of 3 min for ¹³C3-Testosterone was not detected at concentrations lower than 500 ng/mL. Additionally, the signal area at higher concentration, e.g. 500 ng/mL and 1000 ng/mL, was very low and hardly detectable. A reason for this behavior in contrast to the analysis in neat solution matrix can be the analyte suppression in the ionization process by matrix molecules.

The signals for Mz2974 and Girard T-derivatized Testosterone were detected over the full concentration range. Even at very low analyte concentrations where ¹³C₃-Testosterone was not detectable directly, the derivatized testosterone showed clearly a corresponding signal. Compared to the findings in neat solution matrix, the signal areas in the MeOH-depleted horse serum matrix were generally lower. Especially, Girard T-derivatized testosterone was detectable in MeOH-depleted horse serum matrix at very low concentrations from 0.01 ng/mL-0.5 ng/mL by static nanoESI injection.

In this experiment, the analysis of the results was evaluated by the signal area over a time period of 3 minutes instead of using the internal standard ratio. Unfortunately, the internal standard n-Decylbenzamide at 100 ng/mL was suppressed by matrix molecules. Nevertheless, the successful principle of higher signal intensity by derivatization was shown and future evaluations will use an updated concentration of an internal standard.

FIG. 2B shows the results of these two methods. It is shown the relative intensity and areas, respectively, as a function of the concentration of underivatized Testosterone and derivatized Testosterone in MeOH-depleted horse serum matrix. As a derivatizing reagent Girard T and Mz2974 were used. Underivatized ¹³C₃-Testosterone is not or marginal detectable in matrix solution. The derivatised analyte of interest in the pretreated sample leads to an increasing of the sensitivity. Data analysis was performed by the summed area of the signals for a time period of 3 min. Due to ion suppression, the internal standard ratio was not used in this case.

### Example 3: Derivatization. Dilution, and Analysis or Analyte in MeOH-Depleted Horse Serum

Protein Precipitation in Horse Serum:

The horse serum matrix (Sigma, H₀₁₄₆) was precipitated by addition of ice-cold methanol (−20° C.) in the ratio 1:5, mixed on a vortex mixer and subsequently centrifuged for 15 min at 5300 rpm (centrifuge Heraeus Megafuge 16R, Thermo Scientific). The supernatant was transferred and stored at −20° C. until usage.

Derivatization in MeOH-Depleted Horse Serum:

¹³C₃-Testosterone was spiked in MeOH-depleted horse serum matrix, as well as in a Bead Eluat solution in concentrations between 0.04-4000 ng/mL separately. A blank sample was prepared without addition of ¹³C₃-Testosterone. Additionally, for each calibrator a blank reaction was performed by pipetting 50 μL of acetonitril/H2O 50/50 instead of the addition of derivatization reagent.

Afterwards, 50 μL of the respective ¹³C₃-Testosterone calibrator was spiked with 50 μL citric acid (4M), 50 μL m-phenylendiamine (400 mM), and 50 μL of the derivatization reagent. In this derivatization/dilution step, the concentration of ¹³C₃-Testosterone was diluted in the ratio 1:4. Subsequently, the derivatization mixture was shaken for a reaction time of 4 min at 85° C. Accordingly, each calibrator was diluted with a mixture of acetonitrile/H2O 90/10+0.1% formic acid in a ratio of 1:100 and analyzed by Triversa Nanomate nanoESI ionization source and the LTQ mass spectrometer.

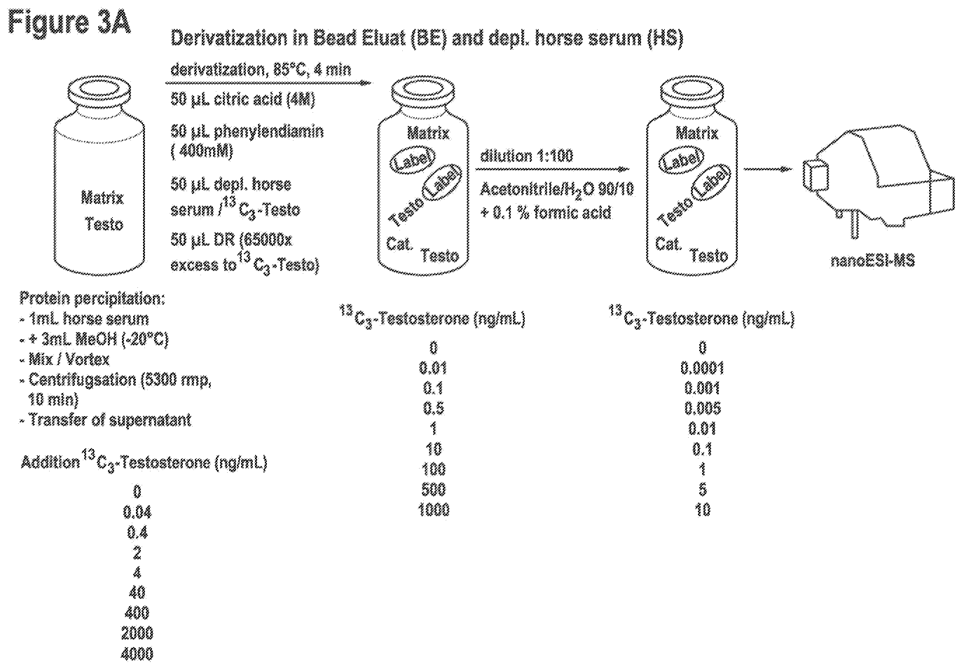

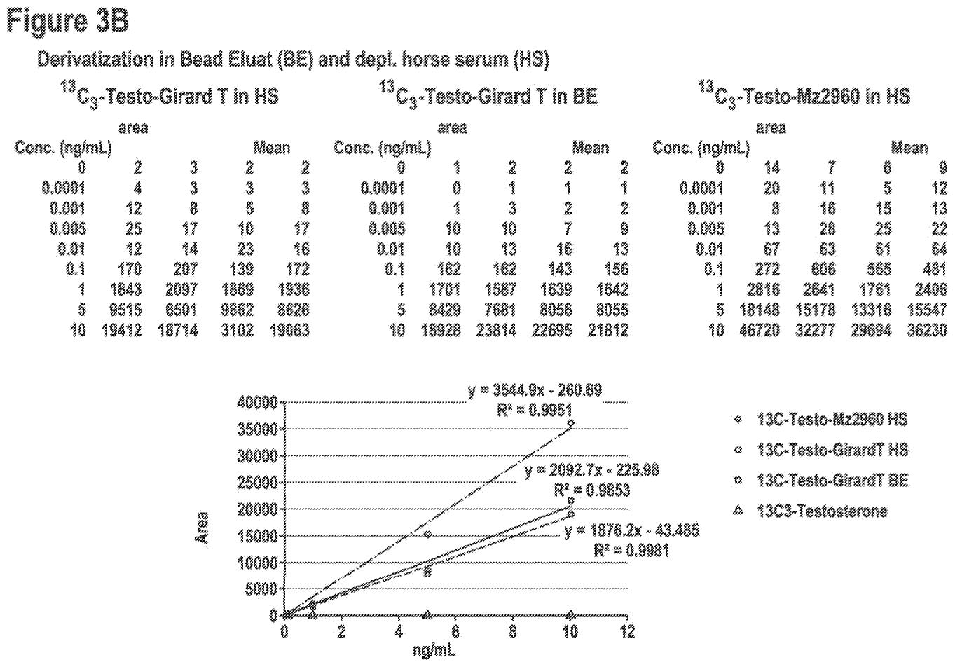

FIG. 3A shows the schematic description of the analyte derivatization followed by further dilution step. Distinct volumes of ¹³C₃-Testosterone are spiked into MeOH-depleted horse serum matrix to result in concentrations varying between 0 and 4000 ng/mL. The derivatization reaction of the analyte is carried out e.g. for 4 min at 85° C. After derivatization the mixture is diluted in the ratio 1:100 and measured by nanoESI mass spectrometry. The derivatising step follows before the diluting step. Additionally, citric acid (e.g. 50 μl, 4 M), m-phenylendiamine (50 μl, 400 mM), depl. horse serum/¹³C₃-Testosterone (50 μl) and the derivatization reagent (50 μl) can be added in the derivatising step. No stable and/or detectable signal of pre-derivatization diluted samples can be observed. The diluting step can be e.g. performed in in acetonitrile/H2O (90:10) and 0.1% formic acid (FA).

Results:

All blank reactions showed no signals at the corresponding m/z ratios. Non-derivatized ¹³C₃-Testosterone showed no signal and was strongly suppressed by the matrix. Even at higher concentrations of 10 ng/mL, ¹³C₃-Testosterone showed no constant signal.

| ¹³C₃-Testosterone | | |
|---|---|---|
| (ng/mL) | area | Mittelwert |

| | | | | |
|---|---|---|---|---|
| 0.01 | 0 | 0 | 0 | 0 |
| 0.1 | 0 | 0 | 0 | 0 |
| 1 | 0 | 0 | 0 | 0 |
| 5 | 1 | 2 | 1 | 1 |
| 10 | 2 | 0 | 3 | 1 |

The derivatization product of ¹³C₃-Testosterone and Girard T was constantly detected in low concentrations at 0.1 ng/ml, in MeOH-depleted horse serum as well in bead eluat matrix. At initial ¹³C₃-Testosterone concentrations lower 0.1 ng/mL, the signal intensity was not permanently high. Presumably, the limit of detection for this analyte is located in this concentration range. Girard T-derivatized ¹³C-Testosterone showed similar results in both matrix systems.

The derivate of ¹³C₃-Testosterone and Mz2960 was analyzed in MeOH-depleted horse serum matrix only. In comparison to the Girard T-derivate, the Mz2960-derivate showed a higher intensity at comparable initial ¹³C₃-Testosterone concentrations. Likewise, the Mz2960-Testosterone derivate was detected constantly at low concentrations of 0.1 ng/mL. All calibrators showed a linear dependency in the measured concentration range. The structure of Mz2960 is:

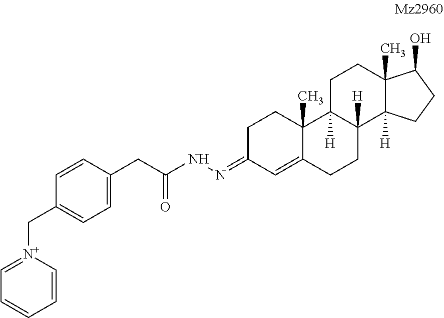

FIG. 3B shows the results of the derivatization of ¹³C₃-Testosterone with Girard T in MeOH-depleted horse serum and Bead Eluat as well as the derivatization with Mz2960 in MeOH-depleted horse serum and subsequent dilution of the analyte mixtures.

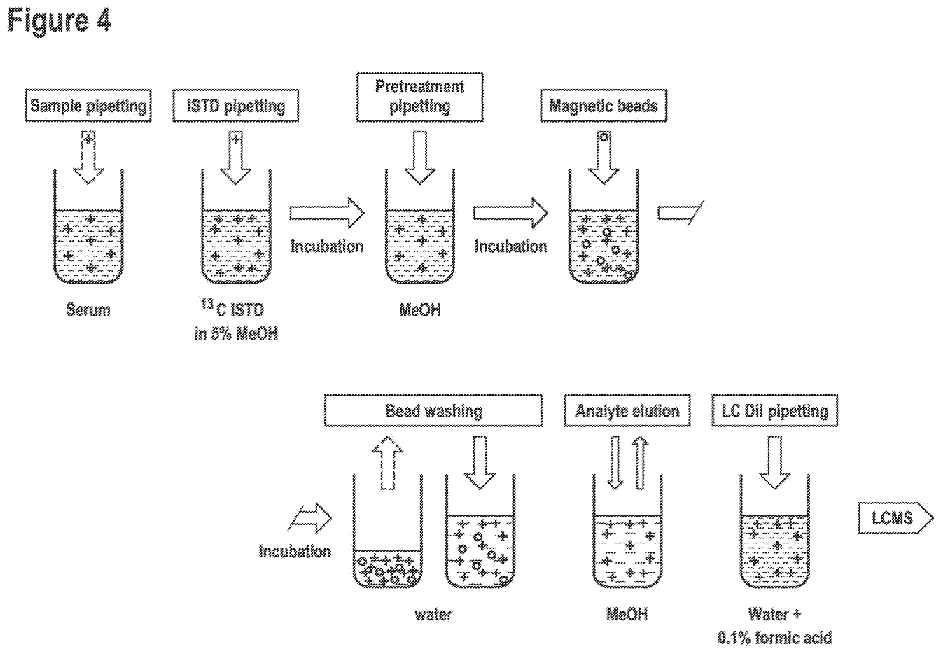

FIG. 4 shows an enrichment step according to the present invention. The serum sample is pipetted into a vessel. Accordingly, the internal standard (ISTD, e.g. a ¹³C-labelled analyte solved in 5% methanol) is added to the sample. After an incubation time, MeOH is added to the sample for pretreatment. After another incubation time, the magnetic bead particles are added to the sample solution and the mixture is incubated for a defined time. Afterwards, the bead/sample-mixture is washed two times with water. Analyte elution is performed by adding a distinct volume of MeOH. Finally, water+0.1% formic acid is added and the sample mixture is ready for analysis.

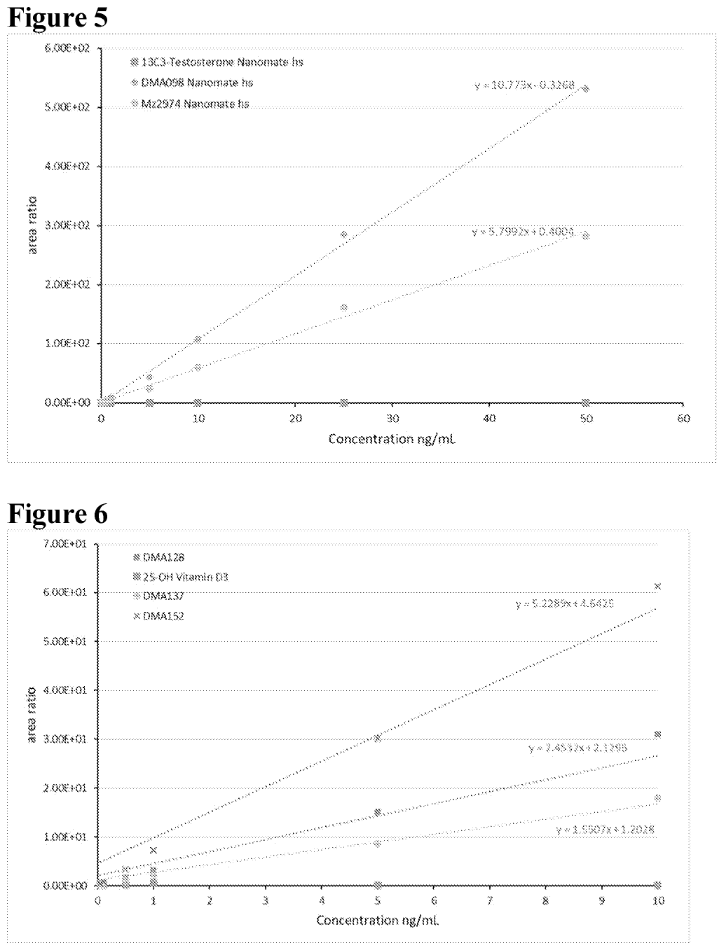

FIG. 5 shows the area ratio as a function of the concentration in ng/ml of a ¹³C₃-Testosterone and the derivatives DMA098 or Mz2974 in depl. horse serum according to a comparative method by using nanoESI, preferably static nanoESI (Nanomate hs) instead of ESI, preferably static ESI. The spiked ¹³C₃-Testosterone is not detectable in depl. horse serum. In contrast to ¹³C₃-Testosterone, DMA098 (Gir. T derivate) and Mz2974 show a higher area ratio and high linearity at the selected concentration range. Derivatization of the analyte and measurement by nanoESI allows a quantification of the analyte at low concentration ranges

The structure of DMA098 is.

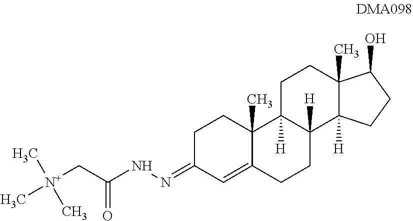

FIG. 6 shows the area ratio as a function of the concentration in ng/ml of DMA128. 25-OH Vitamin D3, DMA137 and DMA152 in depletion (depl.) horse serum according to a comparative method by using nanofESI (Nanomate hs), preferably static nanoESI instead of ESI, preferably static ESI The spiked 25-OH Vitamin D3 is not detectable in depl. horse serum. In contrast. DMA128 (E2 derivate), DMA137 and DMA152 (25-OH Vit D3 derivates) show a higher area ratio and high linearity at the selected concentration range. Derivation of the analyte and measurement by nanoESI allows a quantification of the analyte at low concentration ranges. The structures of DMA128, DMA137, DMA152 and 25-OH Vitamin D3 are:

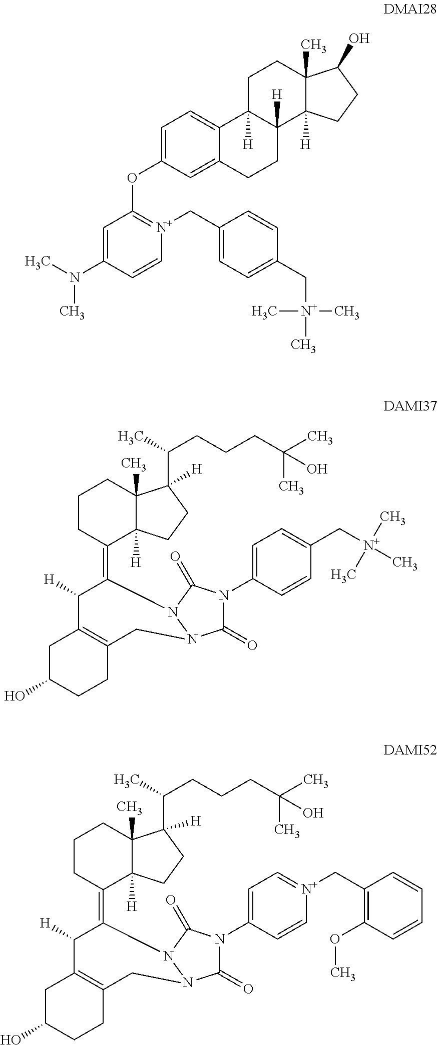

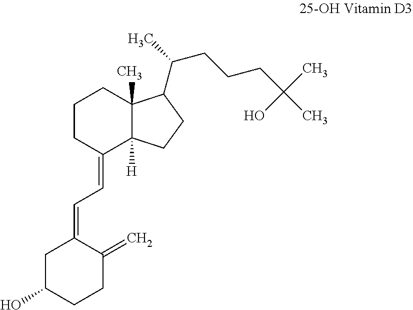 [SMILES: C=C1CC[C@H](O)C/C1=C/C=C1\CCC[C@]2(C)C([C@H](C)CCCC(C)(C)O)CC[C@@H]12]

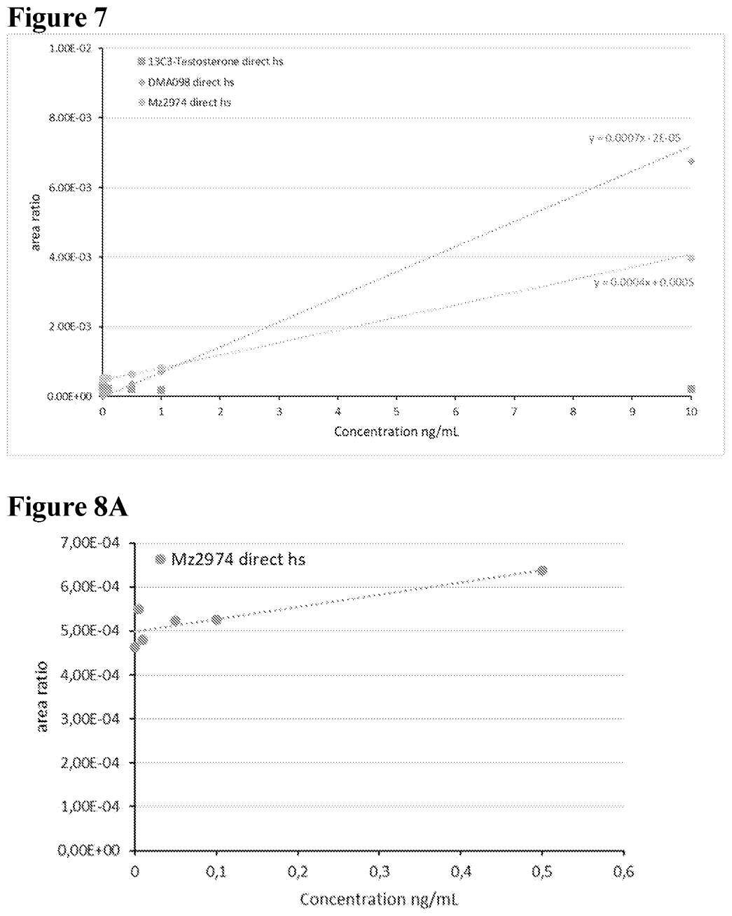

FIG. 7 shows the area ratio as a function of the concentration in ng/ml of ¹³C₃-Testosterone and the derivatives DMA098 or Mz2974 in depiction horse serum according to a method by using ESI, preferably static ESI (direct injection, 100 μL/min). The spiked ¹³C₃-Testosterone is not detectable in depl. horse serum. High matrix background and less ionization efficiency of ¹³C₃-Testosterone leads to depressed signal compared to labeled versions of Testosterone DMA098 (Gir. T derivate) and Mz2974 show higher signal intensities and linearity allowing a quantisation at the low concentration range.

FIGS. 8A and 8B show the comparison of nanoESI (Nanonmate. ˜0.5 μL/min), preferably static nanoESI, and ESI (direct injection, 100 μL/min), preferably static ESI, of Mz2974 in depl. horse serum. It is shown the area ratio as a function of the concentration in ng/ml. FIG. 8A shows high matrix background and signal depression in direct injection. The limit of detection (LOD) of 0 21 ng/ml is estimated according to DIN 32645 as first approximation. Compared to that FIG. 83 shows higher linearity and sensitivity at same concentrations. The limit of detection (LOD) of 0.05 ng/ml is estimated according to DIN 32645 as first approximation. This means a LOD factor of 0.21/005=4.2 Nanospray ionization of the derivatized analyte roughly shows a 4 times higher sensitivity than Electrospray Ionization at higher flowrates (e.g. 100 μL/min).

FIGS. 9A and 9B show the comparison of nanoESI (Nanomate, ˜0.5 μL/min), preferably static nanoESI, and ESI (direct injection, 100 μL/min), preferably static ESI, of DMA098 in depl. horse serum. It is shown the area ratio as a function of the concentration in ng/ml. FIG. 9A shows high matrix background and signal depression in direct injection. The limit of detection (LOD) of 0.10 ng/ml is estimated according to DIN 32645 as first approximation. Compared to that FIG. 9B shows higher linearity and sensitivity at same concentrations. The limit of detection (LOD) of 0.03 ng/ml is estimated according to DIN 32645 as first approximation. This means a LOD factor of 0.10/0 03=3 3 Nanospray ionization of the derivatized analyte roughly shows a 3 times higher sensitivity than Electrospray Ionization at higher flowrates (e g. 100 μL/min).

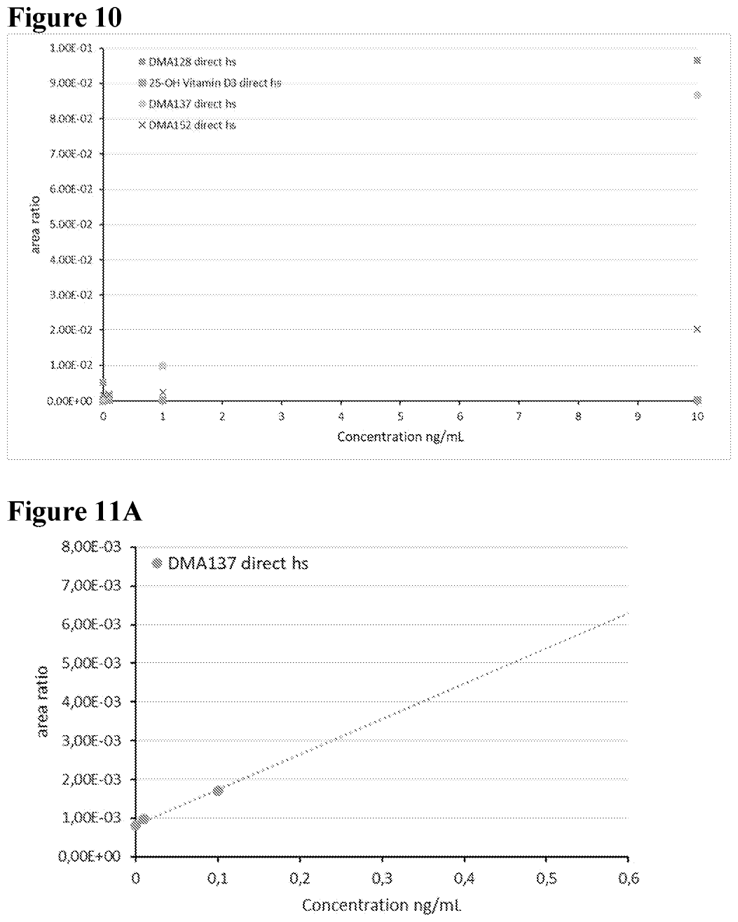

FIG. 10 shows the area ratio as a function of the concentration in ng/ml of DMA128. 25-OH Vitamin D3. DMA137 and DMA152 in depletion horse serum according to a method by using ESI (direct injection, 100 μl/min), preferably static ESI. The spiked 25-011 Vitamin D3 is not detectable in depl horse serum High matrix background and less iontzation efficiency of 25-011 Vitamin D3 leads to depressed signal compared to labelled versions of 25-011 Vitamin D3. DMA128 (E2 derivate). DMA137 and DMA152 (Vit.D3 derivates) show higher signal intensities and lineatin at the concentration range than the non-derivatized analytes.

FIGS. 11A and 11B show the comparison of nanoESI (Nanomate, −0.5 μL/min), preferably static nanoESI, and ESI (direct injection, 100 μL/min), preferably static ESI, of DMA137 in depl. horse serum. It is shown the area ratio as a function of the concentration in ng/ml. FIG. 11A show % s high matrix background and signal depression in direct injection. The him it of detection (LOD) of 0.08 ng/ml is estimated according to DIN 32645 as first approximation. Compared to that FIG. 11B shows higher linearity and sensitivity at same concentrations. The limit of detection (LOD) of 0.03 ng/ml is estimated according to DIN 32645 as first approximation. This means a LOD factor of 0.08/0.03=2.6 Nanospray Ionization of the derivatized analyte roughly shows a 3 times higher sensitivity than Electrospray Ionization at higher flowrates (e.g 100 μL/min).

FIGS. 12A and 12B show the comparison of nanoESI (Nanomate, ˜0.5 μL/min), preferably static nanoESI, and ESI (direct injection, 100 μL/min), preferably static ESI, of DMA152 in depl. horse serum. It is shown the area ratio as a function of the concentration in ng/ml. FIG. 12A shows high matrix background and signal depression in direct injection. The limit of detection (LOD) of 0 079 ng/ml is estimated according to DIN 32645 as first approximation Compared to that FIG. 12B shows higher linearity and sensitivity at same concentrations. The limit of detection (LOD) of 0.04 ng/ml is estimated according to DIN 32645 as first approximation. This means a LOD factor of 0 790.04=10.7. Nanospray ionization of the derivatized analyte roughly shows a 20 times higher sensitivity than Electrospray lionization at higher flowrates (e g 100 μL/min).

FIGS. 13A and 13B show the comparison of nanoESI (Nanomate, ˜0.5 μL/min), preferably static nanoESI, and ESI (direct injection, 100 μL/min), preferably static ESI, of DMA128 in depl. horse scrum. It is show % n the area ratio as a function of the concentration in ng/ml. FIG. 13A shows high matrix background and signal depression in direct injection. The limit of detection (LOD) of 0.070 ng/ml is estimated according to DIN 32645 as first approximation. Compared to that FIG. 13B shows higher linearity and sensitivity at same concentrations. The limit of detection (LOD) of 0.01 ng/ml is estimated according to DIN 32645 as first approximation. This means a LOD factor of 0.70/0.01=70 Nanospray ionization of the derivatized analyte roughly shows a 70 times higher sensitivity than Electrospray Ionization at higher flowrates (e.g. 100 μL/min).

FIG. 14 shows the area ratio as a function of the concentration in ng/ml of different concentrated ¹³C₃-Testosterone (dilution steps 1:10, 1:100, 1:1000) in depletion horse serum according to a method by using nanoESI ¹³C₃-Testosterone calibration curve shows high linearity over all dilution steps.

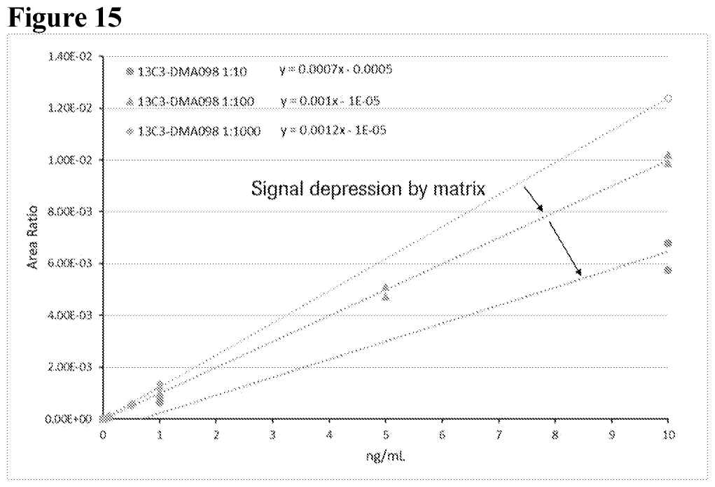

FIG. 15 show % s the area ratio as a function of the concentration in ng/ml of different concentrated ¹³C₃-Testosterone-DMA098 (dilution steps: 1:10, 1:100, 1.1000) in depletion horse serum according to a method by using nanoESI (calibration curve). It is shown, that the highest dilution factor of 1:1000 results in the highest slope of the respective calibration curves. Higher matrix effects in the lower dilution factors 1.10 and 1:100 lead to a signal depression in form of a flattened slope.

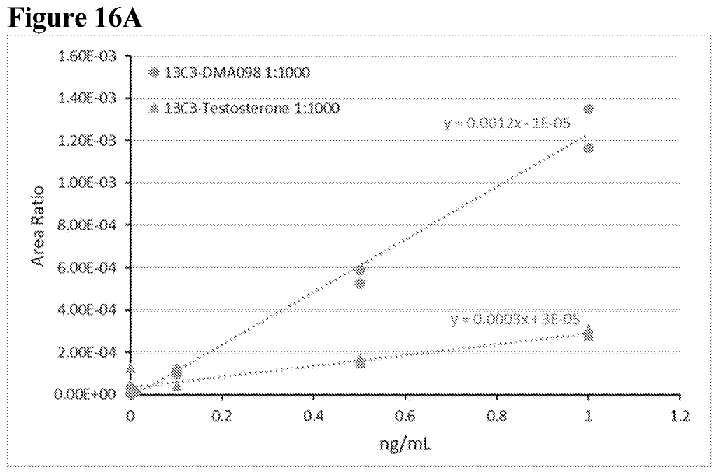

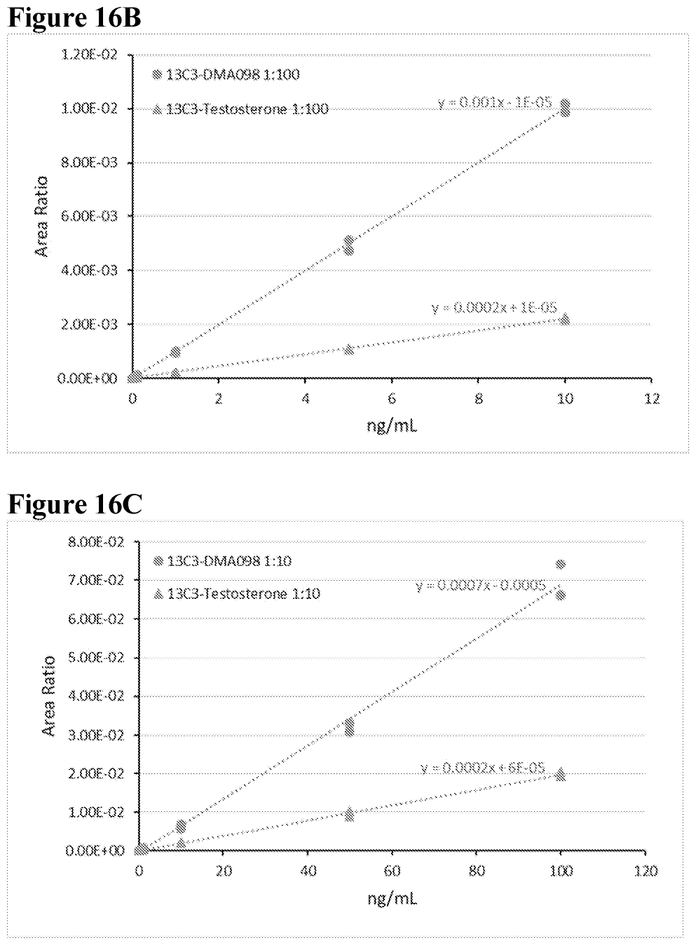

FIG. 16A to 16C show calibration curves of the area ratio as a function of the concentration in ng/ml. of ¹³C₃-Testosterone and derivatized ¹³C₃-Testosterone (DMA098), respectively. At all dilution factor of 1:10, 1.100, and 1:1000, the derivatized form of ¹³C₃-Testosterone-DNA098 shows a higher slope and signal intensity compared to non-derivatized C %-Testosterone.

This patent application claims the priority of the European patent application 20203220.7, w % herein the content of this European patent application is hereby incorporated by references.

1. A method of determining the level of an analyte of interest in a pretreated sample comprising the following steps:
a) providing the pretreated sample,
b) derivatizing the analyte of interest,
c) diluting the pretreated sample, and
d) determining the level of the analyte of interest in the pretreated sample using nanoESI mass spectrometry,
wherein the pretreated sample is a sample of bodily fluid including the analyte of interest.

2. The method of claim 1, wherein the method is free of a further step after performing step a) or step b), wherein the further step is selected from the group consisting of an extraction step, a chromatographic step, lyophilization, centrifuge or combinations thereof.

3. The method of claim 1, wherein the method is automated.

4. The method of claim 1, wherein the method is an in vitro method.

5. The method of claim 1, wherein the pretreated sample is a hemolysed whole-blood sample.

6. The method of claim 1, wherein the analyte of interest is derivatized in step b) by a compound, which is capable of forming a covalent binding to the analyte of interest.

7. The method of claim 6, wherein the compound comprises a permanent charge, wherein said compound is capable of covalently binding to the analyte of interest,
wherein said compound has a mass m1 and a net charge z1,
wherein the compound is capable of forming at least one daughter ion having a mass m2<m1 and a net charge z2<z1 after fragmentation by mass spectrometric determination, and
wherein m1/z1<m2/z2.

8. The method of claim 6, wherein the compound is selected from the group consisting of dansylchloride, carbamic acid, N-[2-[[[2-(diethylamino)ethyl]amino]carbonyl]-6-quinolinyl]-, 2,5-dioxo-1-pyrrolidinyl ester (RapiFluor-MS), 4-substituted 1,2,4-triazoline-3,5-diones (Cookson-type reagents), 4-phenyl-1,2,4-triazolin-3,5-dion-derivative (Amplifex Diene), 1-propanaminium, 3-(aminooxy)-N,N,N-trimethyl-compound comprising an appropriate counter ion (Amplifex Keto), acethydrazide trimethylammonium chloride (Girard T), 1-(carboxymethyl)pyridinium chloride hydrazide (Girard P) and pyridiyl amine.

9. The method of claim 6, comprising the compound of formula A or B: 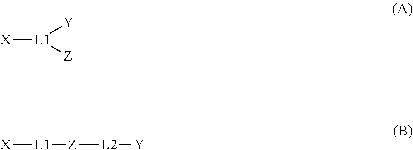
wherein
X is a reactive unit, which is capable of forming a covalent bond with an analyte of interest,
L1 and L2 are independently of each other substituted or unsubstituted linker,
Y is a neutral loss unit, and
Z is a charged unit comprising at least one permanently charged moiety,
including any salt thereof, and/or
comprising the compound of formula P1: 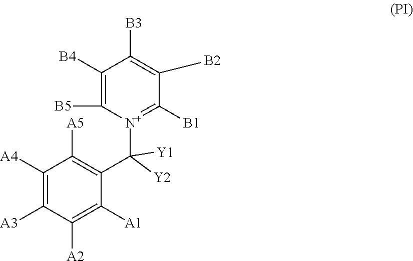
wherein one of the substituents B1, B2, B3, B4, B5 is a coupling group Q, which is capable of forming a covalent bond with the analyte,
wherein the other substituents A1, A2, A3, A4, A5, B1, B2, B3, B4, B5 are each independently selected from hydrogen, halogen, alkyl, N-acylamino, N,N-dialkylamino, alkoxy, thioalkoxy, hydroxy, cyano, alkoxycarbonyl, alkoxythiocarbonyl, acyl, nitro, thioacyl, aryloyl, fluoromethyl, difluoromethyl, trifluoromethyl, trifluoroethyl, cyanomethyl, cyanoethyl, hydroxyethyl, methoxyethyl, nitroethyl, acyloxy, aryloyloxy, cycloalkyl, aryl, heteroaryl, heterocycloalkyl, amino, isotope or derivative thereof,
wherein Y1 and Y2 are each independently selected from hydrogen, methyl, ethyl, methoxy, substituted aromatic, unsubstituted aromatic, substituted cycloalkyl, unsubstituted cycloalkyl, substituted heteroaromatic, unsubstituted heteroaromatic, amine or wherein Y1 and Y2 form a ring structure, which is selected from substituted cycloalkyl, unsubstituted cycloalkyl, substituted aromatic, unsubstituted aromatic, substituted heteroaromatic, unsubstituted heteroaromatic, and/or
comprising the compound of formula D1: 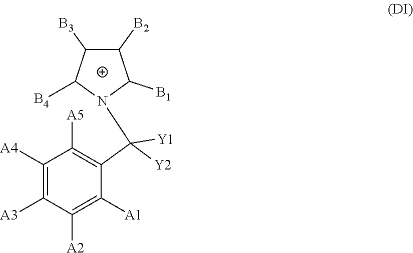
wherein one of the substituents B1, B2, B4 is a coupling group Q, which is capable of forming a covalent bond with the analyte,
wherein the other substituents A1, A2, A3, A4, A5, B1, B2, B4 are each independently selected from hydrogen, halogen, alkyl, N-acylamino, N,N-dialkylamino, alkoxy, thioalkoxy, hydroxy, cyano, alkoxycarbonyl, alkoxythiocarbonyl, acyl, nitro, thioacyl, aryloyl, fluoromethyl, difluoromethyl, trifluoromethyl, trifluoroethyl, cyanomethyl, cyanoethyl, hydroxyethyl, methoxyethyl, nitroethyl, acyloxy, aryloyloxy, cycloalkyl, aryl, heteroaryl, heterocycloalkyl, amino, isotope or derivative thereof,
wherein B3 is selected from alkyl, acetyl, vinyl, substituted aromatic, unsubstituted aromatic, substituted benzyl, unsubstituted benzyl, substituted cycloalkyl, unsubstituted cycloalkyl, isotope and derivative thereof,
wherein Y1 and Y2 are each independently selected from hydrogen, methyl, ethyl, methoxy, substituted aromatic, unsubstituted aromatic, substituted cycloalkyl, unsubstituted cycloalkyl, substituted heteroaromatic, unsubstituted heteroaromatic, amine or wherein Y1 and Y2 form a ring structure, which is selected from substituted cycloalkyl, unsubstituted cycloalkyl, substituted aromatic, unsubstituted aromatic, substituted heteroaromatic, unsubstituted heteroaromatic, and/or
comprising the compound of formula C1: 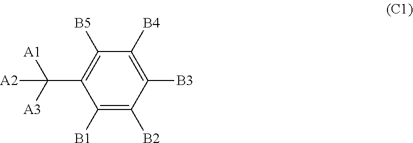
wherein one of the substituents B1, B2, B3, B4, B5 is a coupling group Q, which is capable of forming a covalent bond with the analyte,
wherein the other substituents A1, A2, B1, B2, B3, B4, B5 are each independently selected from hydrogen, halogen, alkyl, modified alkyl, N-acylamino, N,N-dialkylamino, alkoxy, thioalkoxy, hydroxy, cyano, alkoxycarbonyl, alkoxythiocarbonyl, acyl, nitro, thioacyl, aryloyl, fluoromethyl, difluoromethyl, trifluoromethyl, trifluoroethyl, cyanomethyl, cyanoethyl, hydroxyethyl, methoxyethyl, nitroethyl, acyloxy, aryloyloxy, cycloalkyl, aryl, heteroaryl, heterocycloalkyl, amino, sulfur, isotope or derivative thereof,
wherein A3 comprises ammonium, pyridinium, phosphonium or derivatives thereof,
wherein in case of A3 is ammonium and B1 or B5 is the coupling group Q, the coupling group Q comprises a C atom, which is separated by four single or double bonds from the C atom of the CA1A2A3 substituent and the coupling group Q comprises a C-atom, which is separated by five single or double bonds from the C atom of the CA1A2A3 substituent.

10. The method of claim 1, wherein the nanoESI mass spectrometry is static.

11. (canceled)

12. A diagnostic system for determining the level of an analyte of interest in a pretreated sample, comprising a nanoESI source and a mass spectrometer to carry out the method of claim 1.

13. (canceled)

14. A kit suitable to perform the method of claim 1 comprising
(i) a compound for derivatizing the analyte of interest in the pretreated sample, wherein the compound is capable of forming a covalent bond to the analyte of interest,
(ii) a solvent or mixtures of solvents for diluting the pretreated sample comprising the derivatized analyte of interest, and
(iii) optionally a catalyst.

15. (canceled)

16. The method of claim 6, wherein after step b) the compound is covalently bounded to the analyte of interest for forming a complex with the analyte of interest.

17. The method of claim 7, wherein the permanent charge comprises a permanent net charge.

18. The method of claim 9, wherein L1 and L2 are independently of each other branched or linear linker.

19. The method of claim 9, wherein Z is a charged unit comprising one permanently charged moiety.

20. The method of claim 5, wherein the pretreated sample is a hemolysed human whole-blood sample.
